(19) United States
(12) Patent Application Publication
(10) Pub. No.: US 2008/0004290 A1
(43) Pub. Date: Jan. 3, 2008

(54) Anti-Cancer Agents

(75) Inventors: David Fairlie, Springwood (AU); Matthew Glenn, Kangaroo Point (AU); Pia Kahnberg, Mondal (SE)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: THE UNIVERSITY OF QUEENSLAND, St. Lucia (AU)

(21) Appl. No.: 10/596,074

(22) PCT Filed: Nov. 26, 2004
(86) PCT No.: PCT/AU04/01667

§ 371 Date: Jan. 25, 2007

(30) Foreign Application Priority Data
Nov. 28, 2003 (AU) 2003906633

(51) Int. Cl. A61K 31/166; A61K 31/341; A61K 31/381; A61K 31/404; A61K 31/4406; A61K 31/4468; A61K 31/47; A61K 31/4965; C07C 235/14; C07C 323/47; C07D 209/18; C07D 209/42; C07D 211/58; C07D 213/75; C07D 215/40; C07D 241/24; C07D 307/68; C07D 333/38

(52) U.S. Cl. 514/255.06; 514/311; 514/331; 514/352; 514/415; 514/448; 514/471; 514/616; 544/406; 546/176; 546/244; 546/309; 548/491; 549/505; 549/72; 564/153

(57) ABSTRACT

The present invention provides compounds having the structural formula (I): 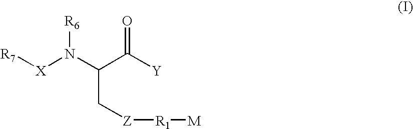 and methods for the treatment of cancer using compounds of formula (I).

### FIELD OF THE INVENTION

The present invention relates to anti-proliferative and anti-cancer agents, particularly those anti-cancer agents that have a core framework structurally related to or derived from amino acid or amino acid like frameworks such as cysteine or 7-substituted 2-amino-heptanoates and which may be utilised in cancer and antiproliferative therapies either on their own or in combination with other anti-cancer agents. The invention further provides pharmaceutical and/or veterinary compositions containing the anti-cancer agents of the invention that may be used in the treatment of cancers. The invention further relates to the use of the anti-cancer agents of the invention in the preparation of medicaments for the treatment of cancer and to methods of treatment of cancer using the anti-cancer agents or compositions containing them.

### BACKGROUND OF THE INVENTION

Cancer is one of the leading causes of death in the modern world with the incidence of cancer related deaths rising with the ageing population. At the present time there are three main treatment strategies for cancer: (1) removal of the cancer by surgery (where possible), (2) use of radiotherapy, or (3) use of combination chemotherapy. With some cancer types a combination strategy is used in which as much of the cancerous tissue being removed by surgery as possible followed by a course or courses in chemotherapy to eliminate any remaining cancer cells. A major dose-limiting problem associated with most chemotherapy is the general toxicity of the drugs currently available. Anti-cancer drugs today are typically general cytotoxins with little selectivity in their killing action for cancer cells over normal human cell types. This lack of selectivity leads to a significant number of adverse side effects in patients who undergo chemotherapy.

The development of truly selective cancer chemotherapy in which a drug specifically destroys malignant cells without damaging normal cells remains an elusive goal. A further promising strategy (Marks et al., 1994; Rifkind et al., 1996, Leszczyniecka et al., 2001; Vigushin et al., 2002) is the use of agents that can differentiate cancer cells to either a non-proliferating or normal phenotype, an approach that has the potential to be tissue-specific and avoid side effects of current drugs. However, most compounds known to differentiate tumour cancer cells are of low potency in cell culture and tend to be non-selective in vivo, where differentiation is reversible or drug resistance is a problem. A few natural products (e.g. trichostatins (Tsuji et al., 1976; Yoshida et al., 1990) and trapoxins (Kijima et al., 1993)) and close analogues display potent differentiating properties on tumour cells in vitro, but they display little or no selectivity being cytotoxic to both normal and cancer cells and most such compounds are ineffective in vivo due to low bioavailability and rapid metabolism. Representative of the structural formulae of these compounds are Trichostatin A and Trapoxin B as shown below.

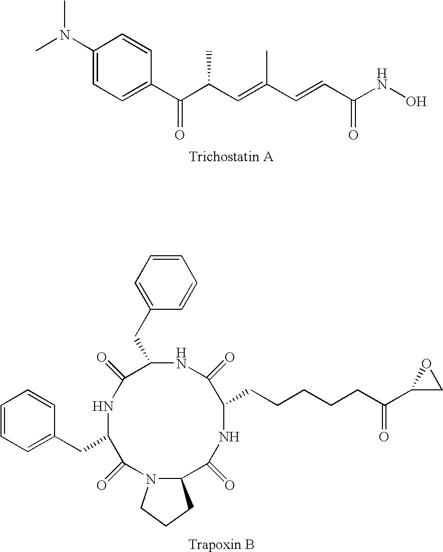

The differentiating agents discussed above are now known to cause hyperacetylation of histones, by inhibiting enzymes known as histone deacetylases (HDACs). It is also clear that multi-protein complexes incorporating HDACs are involved in cell cycle regulation and gene expression. HDACs are involved in modulating chromatin structure by facilitating unpackaging of chromosomal DNA and ‘loosening up’ histones to permit transcription. Histones of the nucleosome are normally tightly wrapped in DNA and linked together, like beads on a string by DNA. Nuclease-mediated digestion of both the linking and wrapping DNA from histones enables gene expression. Unwrapping exposes the octameric histone core, which dissociates into component histones H2A, H2B, H3, H4, etc. Histones are reversibly acetylated on the 6-amino side chain of Lys residues as shown below, and interactions between deacetylated histones and DNA are crucial for gene expression. Histone acetylation and other modifications regulate gene expression by reducing access of transcription factors to DNA. The degree of histone acetylation is regulated by histone acetyl transferases (HATs; 3 groups), deacetylases (HDACs, 16 genes), and their inhibitors, which regulate the cell cycle and consequently hold promise for development of anticancer drugs. Studies by the current applicants and others (WO9855449; Cress et al., 2000; Marks et al., 2001) indicate that HDAC inhibitors cause tumor regression in vivo without damaging DNA.

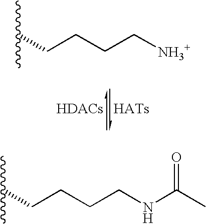

At least eleven HDACs have been identified and, although it is unknown to what extent these enzymes exercise redundant or specific functions, subtle sequence differences between HDACs suggest that it may be possible to develop inhibitors that are selective for specific HDAC enzymes. Crystallographic studies on the histone deacetylase-like protein (HDLP) isolated from Aquifex aeolicus indicate that the active site residues of these enzymes are highly conserved, with most variability at the entrance to this cleft, particularly on the solvent exposed rim of the active site that accommodates the lysine side chain. Furumai et al., (2001) has shown that a carboxylic acid analogue of trapoxin, which is a poorer zinc ligand, is still potent with IC₅₀ of 100 nM probably due to the existence of significant interactions with the protein surface at the entrance to the HDAC active site.

Notwithstanding the potential of the above compounds and analogues thereof as anti-cancer agents, there is the need to develop further potential anti-cancer agents that provide viable alternatives to the known treatments. In particular there is the need to develop anti-cancer agents that have therapeutic efficacy in vivo and which show some degree of selectivity for cancer cells. A further advantage would be obtained if such compounds were also able to revert the transformed morphology of cancer cells to that of a non-proliferating phenotype.

Herein we describe a facile entry to new antitumor compounds designed to reproduce and modify protein surface-binding interactions made by hydrophobic substituents found in highly potent naturally occurring HDAC inhibitors such as trichostatin and trapoxin B. The applicants have conducted investigations to design a consensus structural scaffold for the development of such antitumour agents. The resulting scaffold provides a convenient source of assymetry to append functionality in several directions and is amenable to combinatorial synthesis. The applicants have used toxicity/selectivity for tumor cells as the primary screen to guide the compound development rather than directly measuring inhibition of specific HDACs, since protein acetylation/deacetylation appears to be a general cell signalling device with many protein/DNA targets for HDAC inhibitors. However, because HDAC inhibition does correlate with the potency of the compounds, if not selectivity, a general HDAC-inhibitor pharmacophore has been used to aid the design of active compounds.

The resulting compounds based on the scaffold are cytotoxic antitumour agents that typically inhibit histone deacetylases, cause hyperacetylation of histones, p21 induction, and transform various surviving cancer cells to more normal phenotypes. In particular we describe several compounds derived from the common structural scaffold that demonstrate cytotoxicity selective for proliferative cancer over normal cell lines.

Throughout this specification reference may be made to documents for the purpose of describing various aspects of the invention. However, no admission is made that any reference cited in this specification constitutes prior art. In particular, it will be understood that the reference to any document herein does not constitute an admission that any of these documents forms part of the common general knowledge in the art in Australia or in any other country. The discussion of the references states what their authors assert, and the applicant reserves the right to challenge the accuracy and pertinency of any of the documents cited herein.

### SUMMARY OF THE INVENTION

The present invention provides a compound having the formula (I), or a pharmaceutically acceptable derivative, salt, racemate, isomer or tautomer thereof: 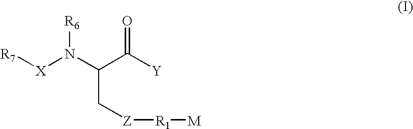
wherein
Z is S or CH₂;
R₁ is a linking moiety;
M is a zinc binding moiety containing at least one heteroatom;
R₆ is selected from the group consisting of H, optionally substituted alkyl, optionally substituted alkenyl, optionally substituted alkynyl and a nitrogen protecting group;
X is selected from the group consisting of: 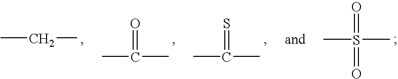
Y is selected from the group consisting: of —NR₄R₅, —OR₄, —SR₄, —CH₂R₄, CHR₄R₅, C(R₄)₂R₅, PHR₄ and PR₄R₅,
wherein R₄ is a group of formula: 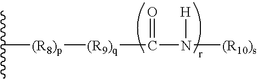
wherein R₈, R₉ and R₁₀ are each independently selected from the group consisting of optionally substituted alkyl, optionally substituted alkenyl, optionally substituted alkynyl, optionally substituted cycloalkyl, optionally substituted aryl, optionally substituted heteroaryl, and optionally substituted heterocycloalkyl;
p, q, r and s are each independently 0 or 1, provided that at least one of p, q ors is 1;
R₅ is H or a group of formula: 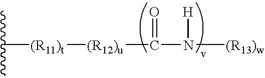
wherein R₁₁, R₁₂ and R₁₁₃ are each independently selected from the group consisting of optionally substituted alkyl, optionally substituted alkenyl, optionally substituted alkynyl, optionally substituted cycloalkyl, optionally substituted aryl, optionally substituted heteroaryl, or optionally substituted heterocycloalkyl;
t, u, v and w are each independently 0 or 1, provided that at least one of t, u and w is 1;
R₇ is a group of formula:
   (R₁₆)_{z}—(R₁₅)_{y}—(R₁₄)ₓ—
wherein R₁₄, R₁₅ and R₁₆ are independently selected from the group consisting of optionally substituted alkyl, optionally substituted alkenyl, optionally substituted alkynyl, optionally substituted cycloalkyl, optionally substituted aryl, optionally substituted heteroaryl and optionally substituted heterocycloalkyl,
x, y and z are independently 0 and 1 with the proviso that at least one of x, y and z is 1.

In one particular embodiment of the invention the compound having the formula (I) is based on cysteine. Accordingly, the embodiment of the invention provides a compound of formula (IIa), or a pharmaceutically acceptable derivative, salt, racemate, isomer or tautomer thereof: 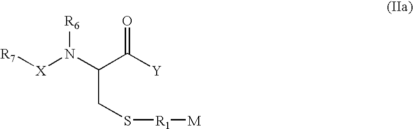
wherein R₁, R₆, R₇, M, X and Y are as defined above for the compound of formula (I).

In another embodiment of the invention the compound having the formula (I) is based on 7-substituted 2-amino-heptanoates. Accordingly, the embodiment of the invention provides a compound of formula (IIb), or a pharmaceutically acceptable derivative, salt, racemate, isomer or tautomer thereof: 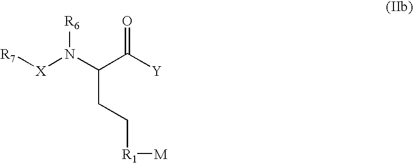
wherein R₁, R₆, R₇, M, X and Y are as defined above for the compound of formula (I).

As with all chemical families there are a number of preferred embodiments within the scope of the general formula. In particular it is preferred that the linking moiety R₁ has between 1 and 9 atoms in a normal chain, preferably between 1 and 4 atoms in a normal chain.

It is also preferred that the group Y is a group of formula —NR₄R₅.

It is preferred that the zinc binding moiety containing a heteroatom is a hydroxamic acid derivative, preferably a group of formula —C(O)NR₂—OR₃ where R₂ is H, optionally substituted alkyl, optionally substituted alkenyl, optionally substituted alkynyl, optionally substituted aryl, or a nitrogen protecting group and R₃ is H, optionally substituted alkyl, optionally substituted alkenyl, optionally substituted alkynyl, optionally substituted aryl or an oxygen protecting group.

Accordingly in a preferred embodiment the present invention provides a compound having the formula (III), or a pharmaceutically acceptable derivative, salt, racemate, isomer or tautomer thereof: 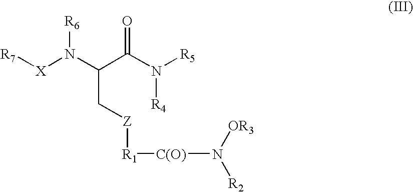
wherein
R₁ is optionally substituted C₁-C₄ alkyl, optionally substituted C₁-C₄ alkenyl or optionally substituted C₁-C₄ alkynyl;
R₂ is H, optionally substituted alkyl, optionally substituted alkenyl, optionally substituted alkynyl, optionally substituted aryl, or a nitrogen protecting group;
R₃ is H, optionally substituted alkyl, optionally substituted alkenyl, optionally substituted alkynyl, optionally substituted aryl or an oxygen protecting group;
R₄ is a group of formula: 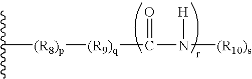
wherein R₈, R₉ and R₁₀ are each independently selected from the group consisting of optionally substituted alkyl, optionally substituted alkenyl, optionally substituted alkynyl, optionally substituted cycloalkyl, optionally substituted aryl, optionally substituted heteroaryl, and optionally substituted heterocycloalkyl;
p, q, r and s are each independently 0 or 1, provided that at least one of p, q or s is 1;
R₅ is H or a group of formula: 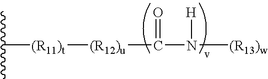
wherein R₁₁, R₁₂ and R₁₁₃ are each independently selected from the group consisting of optionally substituted alkyl, optionally substituted alkenyl, optionally substituted alkynyl, optionally substituted cycloalkyl, optionally substituted aryl, optionally substituted heteroaryl, or optionally substituted heterocycloalkyl;
t, u, v and w are each independently 0 or 1, provided that at least one of t, u and w is 1.
R₆ is selected from the group consisting of H, optionally substituted alkyl, optionally substituted alkenyl, optionally substituted alkynyl and a nitrogen protecting group;
X is selected from the group consisting of 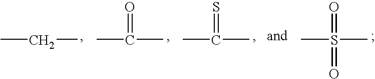
R₇ is a group of formula:
   (R₁₆)_{z}—(R₁₅)_{y}—(R₁₄)ₓ—
wherein R₁₄, R₁₅ and R₁₆ are independently selected from the group consisting of optionally substituted alkyl, optionally substituted alkenyl, optionally substituted alkynyl, optionally substituted cycloalkyl, optionally substituted aryl, optionally substituted heteroaryl and optionally substituted heterocycloalkyl;
x, y and z are independently 0 and 1 with the proviso that at least one of x, y and z is 1.

Even within this preferred subset of compounds there are a number of preferred values for each of the variables in the structural formula given above. For example it is preferred that R₁ is optionally substituted C₁-C₄ alkyl, more preferably optionally substituted C₂-C₃ alkyl, even more preferably optionally substituted C₃ alkyl, most preferably propyl.

It is preferred that R₂ is either H, optionally substituted C₁-C₄ alkyl or a nitrogen protecting group, more preferably H or a nitrogen protecting group, most preferably H.

It is preferred that R₃ is either H, optionally substituted C₁-C₄ alkyl or an oxygen protecting group, more preferably H or an oxygen protecting group, most preferably H.

Particularly preferred compounds of formula (III) are therefore those of formula (IIIa) and (IIIb).

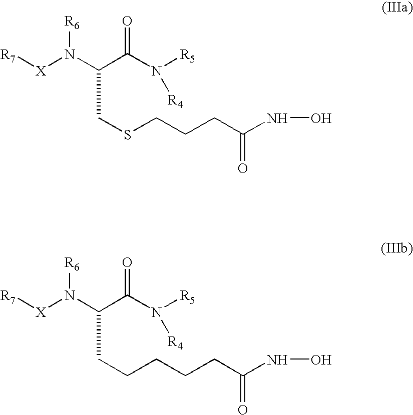

In the compounds of the invention it is preferred that X is a carbonyl group.

It is preferred that R₅ is either H or optionally substituted alkyl, preferably H.

It is preferred that R₆ is either H or a nitrogen protecting group, most preferably H.

In one preferred embodiment the group R₄ is of the formula 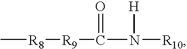
wherein R₈, R₉ and R₁₀ are as defined above.

In this embodiment it is particularly preferred that R₄ is of the formula:

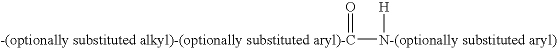

In the most preferred form of this embodiment R₄ is a group of the formula. 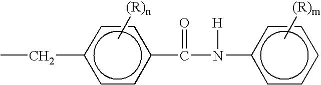
wherein each R is independently selected from the group consisting of alkyl, alkenyl, alkynyl, aryl, heteroaryl, cycloalkyl, heterocycloalkyl, halo, haloalkyl, haloalkenyl, haloalkynyl, haloaryl, haloheteroaryl, halocycloalkyl, haloheterocycloalkyl, hydroxy, alkoxy, alkenyloxy, aryloxy, heteroaryloxy, cycloalkyloxy, heterocycloalkyloxy, benzyloxy, haloalkoxy, haloalkenyloxy, haloaryloxy, halohetoraryloxy, nitro, nitroalkyl, nitroalkenyl, nitroalkynyl, nitroaryl, nitroheteroaryl, nitroheterocyclyoalkyl, amino, alkylamino, dialkylamino, alkenylamino, alkynylamino, arylamino, heteroarylamino, diarylamino, benzylamino, dibenzylamino, acyl, alkenylacyl, alkynylacyl, arylacyl, heteroarylacyl, acylamino, diacylamino, acyloxy, alkylsulphonyloxy, arylsulphonyloxy, heterocycloalkylamino, alkylsulphonyl, arylsulphonyl, carboalkoxy, carboaryloxy, alkylthio, benzylthio, acylthio, cyano, nitro, sulfate and phosphate;
n is 0-4, and
m is 0-5.

In an another preferred embodiment of the invention R₄ is selected from the group consisting of: optionally substituted alkyl, optionally substituted alkenyl, optionally substituted alkynyl, optionally substituted aryl, optionally substituted cycloalkyl, optionally substituted heteroaryl, optionally substituted heterocycloalkyl, optionally substituted arylalkyl, optionally substituted heteroarylalkyl, optionally substituted cycloalkylalkyl, optionally substituted heterocycloalkylalkyl, optionally substituted aryl alkenyl, optionally substituted heteroaryl alkenyl, optionally substituted cycloalkyl alkenyl, optionally substituted heterocycloalkyl alkenyl, optionally substituted aryl alkynyl; optionally substituted heteroaryl alkynyl optionally substituted cycloalkyl alkynyl, optionally substituted heterocycloalkyl alkynyl.

In this embodiment it is particularly preferred that R₄ is selected from optionally substituted aryl, optionally substituted cycloalkyl, optionally substituted heteroaryl, optionally substituted alkyl, optionally substituted arylalkyl, optionally substituted heteroarylalkyl, optionally substituted cycloalkyl alkyl, optionally substituted alkyl aryl, optionally substituted alkyl heteroaryl, optionally substituted alkyl heterocycloalkyl.

In a most preferred embodiment of the invention R₄ has one of the following formulae. 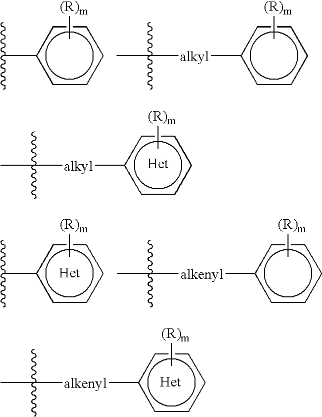
Wherein each R is independently selected from the group consisting of alkyl, alkenyl, alkynyl, aryl, heteroaryl, cycloalkyl, heterocycloalkyl, halo, haloalkyl, haloalkenyl, haloalkynyl, haloaryl, haloheteroaryl, halocycloalkyl, haloheterocycloalkyl, hydroxy, alkoxy, alkenyloxy, aryloxy, heteroaryloxy, cycloalkyloxy, heterocycloalkyloxy, benzyloxy, haloalkoxy, haloalkenyloxy, haloaryloxy, halohetoraryloxy, nitro, nitroalkyl, nitroalkenyl, nitroalkynyl, nitroaryl, nitroheteroaryl, nitroheterocyclyoalkyl, amino, alkylamino, dialkylamino, alkenylamino, alkynylamino, arylamino, heteroarylamino, diarylamino, benzylamino, dibenzylamino, acyl, alkenylacyl, alkynylacyl, arylacyl, heteroarylacyl, acylamino, diacylamino, acyloxy, alklysulphonlyoxy, arylsulphonyloxy, heterocycloalkylamino, alkylsulphonyl, arylsulphnyl, carboalkoxy, carboaryloxy, alkylthio, benzylthio, acylthio, cyano, nitro, sulfate and phosphate;
and each m is from 0-5.

In the compounds of the invention it is preferred that R₇ is selected from the group consisting of optionally substituted alkyl, optionally substituted alkenyl, optionally substituted alkynyl, optionally substituted aryl, optionally substituted cycloalkyl, optionally substituted heteroaryl, optionally substituted heterocycloalkyl, optionally substituted aryl alkyl, optionally substituted heteroaryl alkyl, optionally substituted cycloalkyl alkyl, optionally substituted heterocycloalkyl alkyl, optionally substituted aryl alkenyl, optionally substituted hetero alkenyl, optionally substituted cycloalkyl alkenyl, optionally substituted heterocycloalkyl alkenyl, optionally substituted aryl alkynyl, optionally substituted heteroaryl alkynyl, optionally substituted cycloalkyl alkynyl, and optionally substituted heterocycloalkyl alkynyl.

It is even more preferred that R₇ is optionally substituted aryl, optionally substituted heteroaryl, optionally substituted alkyl, optionally substituted cycloalkyl, optionally substituted heterocycloalkyl, optionally substituted aryl alkyl, optionally substituted alkenyl, and optionally substituted aryl alkenyl.

It is most preferred that R₇ has one of the following formula: 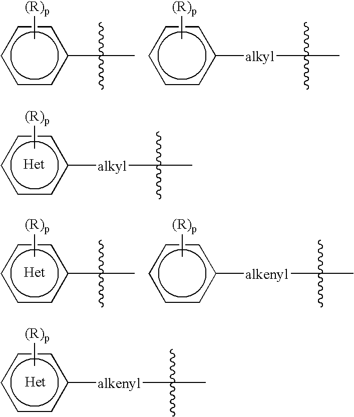
wherein each R is independently selected from the group consisting of alkyl, alkenyl, alkynyl, aryl, heteroaryl, cycloalkyl, heterocycloalkyl, halo, haloalkyl, haloalkenyl, haloalkynyl, haloaryl, haloheteroaryl, halocycloalkyl, haloheterocycloalkyl, hydroxy, alkoxy, alkenyloxy, aryloxy, heteroaryloxy, cycloalkyloxy, heterocycloalkyloxy, benzyloxy, haloalkoxy, haloalkenyloxy, haloaryloxy, halohetoraryloxy, nitro, nitroalkyl, nitroalkenyl, nitroalkynyl, nitroaryl, nitroheteroaryl, nitroheterocyclyoalkyl, amino, alkylamino, dialkylamino, alkenylamino, alkynylamino, arylamino, heteroarylamino, diarylamino, benzylamino, dibenzylamino, acyl, alkenylacyl, alkynylacyl, arylacyl, heteroarylacyl, acylamino, diacylamino, acyloxy, alklysulphonlyoxy, arylsulphonyloxy, heterocycloalkylamino, alkylsulphonyl, arylsulphonyl, carboalkoxy, carboaryloxy, alkylthio, benzylthio, acylthio, cyano, nitro, sulfate and phosphate;
and each p is from 0-5.

A number of specific compounds are particularly preferred. The structures of particularly preferred compounds are described in Tables 1 and 5 (compounds of examples 22-58), Tables 2 and 6 (compounds of examples 59-96), Tables 3 and 7 (compounds of examples 97-102), Table 8 (compounds of examples 103-121) and Tables 4 and 9 (compounds of examples 122-168).

Some preferred compounds include the following:

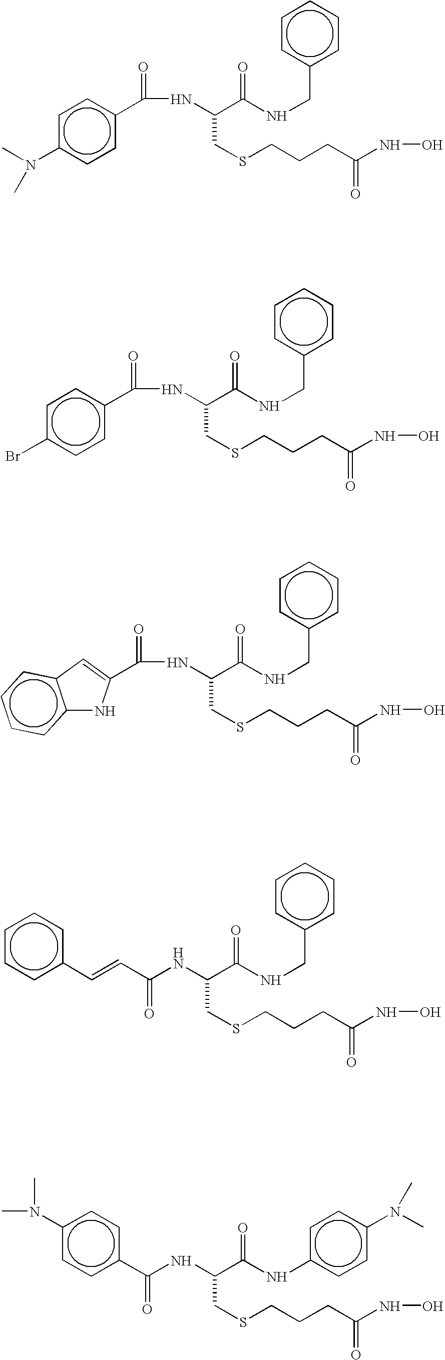

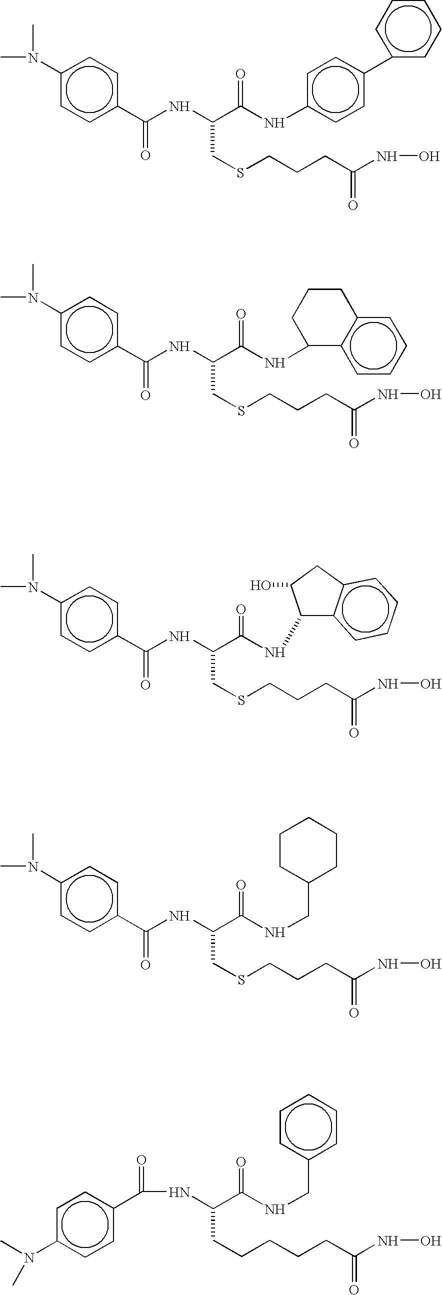

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers.

Particularly preferred compounds are those compounds of formula (III) that have a potency of cytotoxicity of IC₅₀ 10 μM against the MM96 melanoma cells. More preferred are those compounds of formula (III) that have a potency of IC₅₀ 10 μM against the MM96 melanoma cells and a Selectivity Index of 1.5. Even more preferred compounds are those of formula (III) that have a potency of IC₅₀ 10 μM against the MM96 melanoma cells and a Selectivity Index of 3. Most preferred compounds are those of formula (III) that have a potency of IC₅₀ 0.5 μM against the MM96 melanoma cells and a Selectivity Index of 4. Exemplary examples include compounds of examples 24, 40, 48, 59, 66, 67, 100, 123, 124, 125, 126, 130, 131, 132, 133, 137, 138, 146, 148, 160, 162 and 166.

The inventor's studies have shown that compounds of the present invention are cytotoxic anti-cancer agents. Accordingly, the present invention also provides a method for the treatment of cancer in an animal, the method including the step of administering to the animal in need of such treatment an effective amount of a compound having the formula (I), or a pharmaceutically acceptable derivative, salt, racemate, isomer or tautomer thereof: 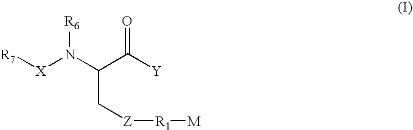
wherein
Z is S or CH₂;
R₁ is a linking moiety;
M is a zinc binding moiety containing at least one heteroatom;
R₆ is selected from the group consisting of H, optionally substituted alkyl, optionally substituted alkenyl, optionally substituted alkynyl and a nitrogen protecting group;
X is selected from the group consisting of: 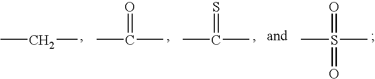
Y is selected from the group consisting: of —NR₄R₅, —OR₄, —SR₄, —CH₂R₄, CHR₄R₅, C(R₄)₂R₅, PHR₄ and PR₄R₅,
wherein R₄ is a group of formula: 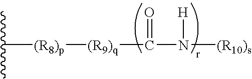
wherein R₈, R₉ and R₁₀ are each independently selected from the group consisting of optionally substituted alkyl, optionally substituted alkenyl, optionally substituted alkynyl, optionally substituted cycloalkyl, optionally substituted aryl, optionally substituted heteroaryl, and optionally substituted heterocycloalkyl;
p, q, r and s are each independently 0 or 1, provided that at least one of p, q or s is 1;
R₅ is H or a group of formula: 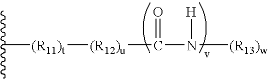
wherein R₁₁, R₁₂ and R₁₁₃ are each independently selected from the group consisting of optionally substituted alkyl, optionally substituted alkenyl, optionally substituted alkynyl, optionally substituted cycloalkyl, optionally substituted aryl, optionally substituted heteroaryl, or optionally substituted heterocycloalkyl;
t, u, v and w are each independently 0 or 1, provided that at least one of t, u and w is 1;
R₇ is a group of formula:
   (R₁₆)_{z}—(R₁₅)_{y}—(R₁₄)ₓ—
wherein R₁₄, R₁₅ and R₁₆ are independently selected from the group consisting of optionally substituted alkyl, optionally substituted alkenyl, optionally substituted alkynyl, optionally substituted cycloalkyl, optionally substituted aryl, optionally substituted heteroaryl and optionally substituted heterocycloalkyl,
x, y and z are independently 0 and 1 with the proviso that at least one of x, y and z is 1.

In a preferred embodiment of the method of the invention the animal is a human. The compound of the invention may be administered in any suitable form well known in the art including oral administration in the form of a liquid, syrup, tablet or the like, by injection or by intravenous infusion. It is preferred that the compound is administered by intravenous infusion.

The present invention also provides pharmaceutical and/or veterinary compositions containing one or more of the compounds of the invention and a pharmaceutically acceptable, carrier, diluent or excipient. These compositions may be used in the methods of treatment discussed previously.

In a further aspect the invention provides the use of the compounds of the invention as hereinbefore described for the preparation of a medicament for the treatment of cancer.

### DESCRIPTION OF THE FIGURES

FIG. 1. Acetylation of Histones. MM96L cells were treated with 5 μg/mL of test compounds for 8 hours, before harvest and analysis of histone H4 acetylation by Triton-acetic acid-urea gel (Saito et al., 1991; Qiu et al., 1999). Lane 1: untreated. Lane 2: compound of example 22 Lane 3: compound of example 40. Lane 4: TSA. Non-acetylated (A), mono-acetylated (B), di-acetylated (C), tri-acetylated (D) and tetra-acetylated (E) histone H4 are indicated.

FIG. 2. Acetylation of histones MM96L cells were treated with 5 μg/ml of various compound for 8 hr, before harvest and analysis of histone H4 acetylation by Triton-acetic acid-urea gel as previously described (Saito et al., 1991; Qiu et al., 1999). Lane 1: untreated: lane 2: Compound of example 73; lane 3: Compound of example 40; lane 4: TSA. Non-acetylated (A), mono-acetylated (B), di-acetylated (C), tri-acetylated (D) and tetra-acetylated (E) histone H4 are indicated.

FIG. 3. Induction of p21 expression. MM96L cells were treated with the compound of example 15 (10 μg/mL) and total RNA was isolated from cells, reverse transcribed using SuperScript II and oligo-dT primer, and cDNA amplified by PCR using primers specific for p21^{WAF1/Cip1} and GAPDH. Lane 1, untreated; lane 2, 16 hours treatment; lane 3, 24 hours treatment; lane 4, RT-PCR negative control. Quantitation of p21^{WAF1/Cip1} induction was performed by densitometric analysis using ImageQuaNT 4.2 software (Molecular Dynamics, Sunnyvale, Calif.) following normalisation to GAPDH product intensity. Expression of p21^{WAF1/Cip1} was increased 2.1-fold above that of untreated cells at both the 16 and 24 hr time points.

FIG. 4. Induction of p21 expression MM96L melanoma cells were treated with 2 compounds at a concentration of 10 μg/ml, and total RNA was isolated following 16 and 24 hrs, as described in Materials and Methods. Semi-quantitative RT-PCR was performed on the total RNA samples. Induction of mRNA for p21^{WAF1/Cip1} was seen after 16 hrs treatment for both compound of example 24 and compound of example 67.

FIG. 5. Morphological Reversion After 24 hours. (a) Untreated normal melanocytes; (b) Normal melanocytes treated with compound of example 40 (10 μg/mL); (c) Untreated melanoma cells (MM96L); (d) MM96L treated with compound of example 40 (10 μg/mL).

FIG. 6. Morphological Reversion After 24 hours. (a) Untreated normal melanocytes; (b) Normal melanocytes treated with compound of example 67 (10 μg/mL); (c) Untreated melanoma cells (MM96L); (d) MM96L treated with compound of example 67 (10 μg/mL).

FIG. 7. Oral Bioavailability. Time dependent plasma concentration of compound of example 24 after oral (top) and intravenous (bottom) administration at 5 mg/kg to each of three Wistar rats.

### DETAILED DESCRIPTION OF THE INVENTION

The compounds of the invention have been found to possess cytotoxic effects against cancer cells and are therefore useful in methods for the treatment of cancer in animals especially humans. As used herein the term ‘cancer’ is a general term intended to encompass the more than 100 conditions that are characterised by uncontrolled abnormal growth of cells.

Examples of cancer types that may be able to be treated by the compounds of the present invention include bone cancers including Ewing's sarcoma, osteosarcoma, chondrosarcoma and the like, brain and CNS tumours including acoustic neuroma, neuroblastomas and other brain tumours, spinal cord tumours, breast cancers, colorectal cancers, endocrine cancers including adenocortical carcinoma, pancreatic cancer, pituitary cancer, thyroid cancer, parathyroid cancer, thymus cancer, multiple endocrine neoplasma, gastrointestinal cancers including stomach cancer, esophageal cancer, Small intestine cancer, Liver cancer, extra hepatic bile duct cancer, gastrointestinal Carcinoid tumour, gall bladder cancer, genitourinary cancers including testicular cancer, penile cancer, prostrate cancer, gynecological cancers including cervical cancer, ovarian cancer, vaginal cancer, uterus/endometrium cancer, vulva cancer, gestational trophoblastic cancer, fallopian tube cancer, uterine sarcoma, head and neck cancers including oral cavity cancer, lip cancer, salivary gland cancer, larynx cancer, hypopharynx cancer, orthopharynx cancer, nasal cancer, paranasla cancer, nasopharynx cancer, leukemias including childhood leukemia, acute lymphocytic leukemia, acute myeloid leukemia, chronic lymphocytic leukemia, chronic myeloid leukemia, hairy cell leukemia, acute promyelocytic leukemia, plasma cell leukemia, myelomas, haematological disorders including myelodysplastic syndromes, myeloproliferative disorders, aplastic anemia, Fanconi anemia, Waldenstroms Macroglobulinemia, lung cancers including small cell lung cancer, non-small cell lung cancer, lymphomas including Hodgkinsons disease, non-Hodgkinsons's lymphoma, AIDS related Lymphoma, eye cancers including retinoblastoma, intraocular melanoma, skin cancers including melanoma, non-melanoma skin cancer, merkel cell cancer, soft tissue sarcomas such as childhood soft tissue sarcoma, adult soft tissue sarcoma, Kaposi's sarcoma, urinary system cancers including kidney cancer, Wilms tumour, bladder cancer, urethral cancer, and transitional cell cancer.

Preferred cancers that may be treated by the compounds of the present invention are melanomas, skin, breast, prostrate and ovarian cancers.

Various terms used throughout the specification have meanings that will be well understood by a skilled addressee in the area. Nevertheless, for ease of reference, some of these terms will now be defined.

The term “animal” as used throughout the specification is to be understood to mean ordinarily a mammal such as a human, sheep, horse, cattle, pig, dog, cats, rat and mouse. For example, the animal may be a human subject suffering the effects of cancer.

The term “alkyl” or “alk” as employed herein alone or as part of another group refers to a monovalent (e.g. -alkyl) or polyvalent (e.g. -alkyl-) saturated hydrocarbon derived radical having the number of carbons specified or if no number is specified up to 30 carbons. The term includes straight or branched saturated hydrocarbon groups. The group preferably contains from 1 to 20 carbons, more preferably from 1 to 10 carbons, even more preferably 1 to 8 carbons in the normal chain. Examples of alkyl include but are not limited to methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, and the various branched chain isomers thereof.

The term “alkene” or “alkenyl” as used herein alone or as part of another group refers to straight or branched unsaturated monovalent (e.g. -alkene) or polyvalent (-alkene-) hydrocarbon radical containing at least one carbon to carbon double bond. The group preferably contains from 2 to 20 carbons, preferably 2 to 12 carbons, most preferably 2 to 8 carbons in the normal chain. The group may include any number of double bonds in the normal chain and the orientation about each double bond is independently E or Z. Examples of alkenyl include but are not limited to ethenyl (vinyl), 2-propenyl, 2-butenyl, 3-butenyl, 3-pentenyl, 4-pentenyl, 2-hexenyl, 3-hexenyl, 2-heptenyl, 3-heptenyl, 3-octenyl, 3-nonenyl, 4-decenyl, 3-undecenyl, 4-dodecenyl, 4,8,12-tetradecatrienyl, and the like.

The term “alkyne” or “alkynyl” as used herein alone or as part of another group refers to a refers to straight, branched or cyclic unsaturated monovalent (e.g. -alkyne) or polyvalent (e.g. -alkyne-) hydrocarbon radical containing at least one carbon to carbon triple bond in the normal chain. The group preferably contains from 2 to 20 carbons, preferably 2 to 12 carbons and more preferably 2 to 8 carbons in the normal chain. Examples of alkynyl include but are not limited to ethynyl, 2-propynyl, 3-buyynyl, 2-butynyl, 3-pentynyl, 4-pentynyl, 2-hexynyl, 3-hexynyl, 2-heptynyl, 3-heptynyl, 4-heptynyl, 2-octynyl, 3-octynyl, 4-octynyl, and the like.

The term “aryl” either alone or part of another group refers to monocyclic, bicyclic, tricyclic or polycyclic aromatic groups preferably containing from 6 to 20 carbons, more preferably from 6 to 14 carbons, even more preferably from 6 to 10 carbons. Examples of aryl include but are not limited to phenyl, 1-naphthyl, 2-naphthyl, anthracyl, phenanthryl, and benzonaphthenyl. These groups may optionally include one to three additional carbocyclic rings fused to the aromatic ring system.

The term “cycloalkyl” alone or as part of another group indicates a saturated or partially unsaturated cyclic hydrocarbon preferably containing from 1 to 3 rings, including monocyclic alkyl, bicyclic alkyl (bicycloalkyl) and tricyclic alkyl (tricycloalkyl), and preferably containing a total of from 3 to 20 carbons forming the ring, preferably 3 to 10 carbons, forming the ring and which may be fused to 1 to 2 aromatic rings. Examples of cycloalkyl include but are not limited to cyclopropyl, cyclobutyl, cyclopentyl, cyclohexyl, cycloheptyl, cyclooctyl, cyclodecyl, adamantyl, and norbornyl

The term “heteroaryl” either alone or part of another group refers to groups containing an aromatic ring (preferably a 5 or 6 membered aromatic ring) having 1 or more heteroatoms as ring atoms in the aromatic ring with the remainder of the ring atoms being carbon atoms. Suitable heteroatoms include oxygen, sulfur, and nitrogen. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, indole, isoindole, 1H-indazole, purine, 4H-quinolidine, isoquinoline, quinoline, phthalazine, naphthyridine, quinoxaline, quinazoline, cinnoline, carbazole, .beta.-carboline, phenanthridine, acridine, phenazine, thiazole, isothiazole, phenothiazine, oxazole, isoxazole, furazane, phenoxazine, 2-, 3-, or 4-pyridyl, 2-, 3-, 4-, 5-, or 8-quinolyl, 1-, 3-, 4-, or 5-isoquinolyl, 1-, 2-, or 3-indolyl, 2-benzothiazolyl, 2-benzo[b]thienyl, benzo[b]furanyl, 2- or 3-thienyl, or the like. More preferred examples include 2- or 3-thienyl, 2-, 3-, or 4-pyridyl, 2- or 3-quinolyl, 1-isoquinolyl, 1- or 2-indolyl, 2-benzothiazolyl, and the like. For ease of reference in the drawings heteroaryl is sometimes depicted with the following symbol.

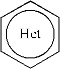

This symbol is intended to be a shorthand notation for all heteroaryl groups whether monocyclic, bicyclic or polycyclic notwithstanding that a single ring is depicted in the shorthand notation.

The term “heterocycloalkyl” as used alone or as part of another group refers to a saturated or partially unsaturated ring, preferably containing 5, 6, 7 or 8 ring atoms which includes at least one of nitrogen, sulfur or oxygen as a ring atom and which may further be fused to one or more aromatic or non-aromatic rings. Examples of heterocycloalkyl include 2-pyrolline, 3-pyrolline, pyrollidine, 1,3 dioxolane, 2-imidazoline, 2-pyrazoline, pyrazolidine, piperidine, morpholine. 1,4-dioxane, thiomorpholine, piperazine and indoline.

The term “acyl” as used throughout the specification is to be understood to mean the groups alkyl-C(O)—, substituted alkyl-C(O)—, cycloalkyl-C(O)—, substituted cycloalkyl-C(O)—, aryl-C(O)—, heteroaryl-C(O) and heterocycloalkyl-C(O)—.

The term “alkoxy” as used throughout the specification is to be understood to mean the group “alkyl-O—”. Preferred alkoxy groups include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

The term “amino” as used throughout the specification is to be understood to mean a nitrogen optionally mono-, di- or tri-substituted.

The terms “halo” or “halogen” as used throughout the specification is to be understood to mean fluoro, chloro, bromo or iodo.

The term “optionally substituted” as used throughout the specification denotes that the group may or may not be further substituted or fused (so as to form a condensed polycyclic system), with one or more substituent groups. Preferably the substituent groups are one or more groups selected from alkyl, alkenyl, alkynyl, aryl, heteroaryl, cycloalkyl, heterocycloalkyl, halo, haloalkyl, haloalkenyl, haloalkynyl, haloaryl, haloheteroaryl, halocycloalkyl, haloheterocycloalkyl, hydroxy, alkoxy, alkenyloxy, aryloxy, heteroaryloxy, cycloalkyloxy, heterocycloalkyloxy, benzyloxy, haloalkoxy, haloalkenyloxy, haloaryloxy, halohetoraryloxy, nitro, nitroalkyl, nitroalkenyl, nitroalkynyl, nitroaryl, nitroheteroaryl, nitroheterocyclyoalkyl, amino, alkylamino, dialkylamino, alkenylamino, alkynylamino, arylamino, heteroarylamino, diarylamino, benzylamino, dibenzylamino, acyl, alkenylacyl, alkynylacyl, arylacyl, heteroarylacyl, acylamino, diacylamino, acyloxy, alklysulphonlyoxy, arylsulphonyloxy, heterocycloalkylamino, alkylsulphonyl, arylsulphonyl, carboalkoxy, carboaryloxy, alkylthio, benzylthio, acylthio, cyano, nitro, sulfate and phosphate;

The term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 2nd Ed. (John Wiley & Sons, Inc., New York). Preferred amino protecting groups include, but are not limited to, benzyloxycarbonyl (CBz), t-butyloxycarbonyl (Boc), t-butyldimethylsilyl (TBDIMS), 9-fluorenylmethyloxycarbonyl (Fmoc), or suitable photolabile protecting groups such as 6-nitroveratryloxy carbonyl (Nvoc), nitropiperonyl, pyrenylmethoxycarbonyl, nitrobenzyl, dimethyl dimethoxybenzil, 5-bromo-7-nitroindolinyl, and the like. Preferred hydroxyl protecting groups include Fmoc, benzyl, t-butyl, allyl, TBDIMS, photolabile protecting groups (such as nitroveratryl oxymethyl ether (Nvom)), Mom (methoxy methyl ether), and Mem (methoxy ethoxy methyl ether). Particularly preferred protecting groups include NPEOC (4-nitrophenethyloxycarbonyl) and NPEOM (4-nitrophenethyloxymethyloxycarbonyl).

The term “Selectivity Index” is used to describe the ratio of compound cytoxic activity, as measured by IC₅₀ values, for normal cells over tumor cells. Unless otherwise specified, the Selectivity Index refers specifically to IC₅₀ (NFF)/IC₅₀ (MM96L). IC₅₀ is a measurement of the concentration of a compound needed to reduce population growth of organisms, including eukaryotic cells, by 50% in vitro. Though often expressed to denote in vitro antibacterial activity, it is also used as a benchmark for cytotoxicity to eukaryotic cells in culture.

As used throughout the specification the preferred number of carbon atoms will be represented by, for example, the phrase “Cₓ-C_{y} alkyl” which refers to an alkyl group as hereinbefore defined containing the specified number of carbon atoms. Similar terminology will apply for other variable.

Pharmaceutically acceptable derivatives and solvates of the compounds of the invention are also contemplated herein. The term “pharmaceutically acceptable derivative” as used throughout the specification is to be understood to mean a compound that is a drug precursor, which, upon administration to a subject, undergoes chemical conversion by metabolic or chemical processes to yield a compound of formula (I) or a salt and/or solvate thereof. The term is used interchangeably with the term ‘prodrug’.

The term “solvate” as used throughout the specification is to be understood to mean a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

The term “composition” as used throughout the specification is to be understood to mean a product containing the specified ingredients in the specified amounts, as well as any product which results, directly or indirectly, from combination of the specified ingredients in the specified amounts.

Log D_{7.0} refers to the lipophilicty of the compounds of the invention and was calculated at pH 7 (Log D_{7.0} being the octanol/water partition coefficient) using the program PALLAS.

The term “therapeutically effective amount” or “therapeutic amount” is an amount sufficient to effect beneficial or desired clinical results. An effective amount can be administered in one or more administrations. An effective amount is typically sufficient to palliate, ameliorate, stabilize, reverse, slow or delay the progression of the disease state.

Compound Design

The compounds were designed on the basis that human histone deacetylases (HDACs) are known to regulate the equilibrium between acetylated and deacetylated nuclear proteins known as histones, and that this control in turn influences the degree of interaction between histones and the DNA in which histones are normally wrapped. One role for histone deacetylases then is to increase the proportion of histones wrapped in DNA, and inhibitors of this enzyme can thus enhance the unwrapping.

The specific molecular interactons between DNA and histones are mediated through lysine side chains of histones. Histone lysines possess side chains consisting of a —(CH₂)₄—NH₂ terminus which when acetylated (—(CH₂)₄—NHCOCH₃), inserts into the active site of HDAC enzymes and makes contact with a zinc ion.

The three dimensional structure of a bacterial HDAC enzyme analogue (HDLP) has been solved both as the native enzyme, and co-crystallized with the HDAC inhibitors trichostatin A and suberoylanilide hydroxamic acid (SAHA). HDLP shares ˜32% homology with HDAC1 and deactetylates histones in vitro. High sequence homology is observed within the hydrophobic tubular catalytic active site, ˜11 Å deep but narrowing to ˜4 Å at the active site and terminating at a divalent zinc cation, activated water molecule, and histidine-aspartate charge-relay system. Most of the residues in the HDLP structure that interact directly with trichlorstatin are highly conserved among all the HDACs, but there is less conservation in adjoining residues, most notably on the enzyme surface which has a number of shallow pockets surrounding the active site channel.

Docking of trapoxin B into the HDLP crystal structure using a combination of conformational searching (MACROMODEL) and a genetic docking algorithm (GOLD) identified tight binding conformations in which the aliphatic side chain had inserted into the tubular pocket of the active site, with the Phe side chains in contact with the shallow pockets of the enzyme surface (Glenn et al., 2004). These aromatic groups represent important foliage on the cyclic tetrapeptide scaffold for tight enzyme binding, and similar groups are represented in related naturally occurring cyclic tetrapeptides (Phe, Trp, Tyr). However, cyclic tetrapeptides offer limited scope for potential therapeutics due to their difficulty of synthesis, problematic stability, and conformational homogeneity. It was generally conceived that active compounds could be developed by mimicking the key enzyme binding regions of Trapoxin B, which would include a zinc chelator tethered to a branched capping group capable of reproducing the approximate positions and orientations of the Phe side chains, on a much simplified template. It was envisaged that a tripeptide incorporating similar surface binding groups to those found in the potent naturally occurring cyclic tetrapeptide inhibitors (hydrophobic, aromatic, basic) would be able to span the surface binding domain of Trapoxin B, while a hydrophobic tether terminating at a hydroxamic acid would ensure firm zinc binding in the catalytic core.

Analysis of the problem led to the conclusion that amino acid like frameworks derived from either cysteine or alpha 7-substituted 2-amino-heptanoate have the potential to meet the above requirements as they provide the appropriate functionality, chirality and orientation to mimic the cyclic peptide, trapoxin B.

Synthetic studies in this area were therefore directed towards the use of cysteine and 7-substituted 2-amino-heptanoate like frameworks as building blocks from which improved compounds could be developed. These studies led to the development of the compounds of the invention.

Thus, the present invention provides a compound having the formula (I), or a pharmaceutically acceptable derivative, salt, racemate, isomer or tautomer thereof: 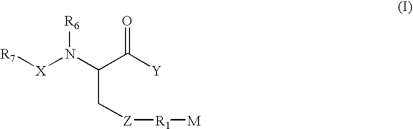
wherein
Z is S or CH₂;
R₁ is a linking moiety;
M is a zinc binding moiety containing at least one heteroatom;
R₆ is selected from the group consisting of H, optionally substituted alkyl, optionally substituted alkenyl, optionally substituted alkynyl and a nitrogen protecting group;
X is selected from the group consisting of: 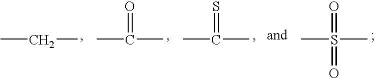
Y is selected from the group consisting: of —NR₄R₅, —OR₄, —SR₄, —CH₂R₄, CHR₄R₅, C(R₄)₂R₅, PHR₄ and PR₄R₅,
wherein R₄ is a group of formula: 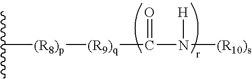
wherein R₈, R₉ and R₁₀ are each independently selected from the group consisting of optionally substituted alkyl, optionally substituted alkenyl, optionally substituted alkynyl, optionally substituted cycloalkyl, optionally substituted aryl, optionally substituted heteroaryl, and optionally substituted heterocycloalkyl;
p, q, r and s are each independently 0 or 1, provided that at least one of p, q or s is 1;
R₅ is H or a group of formula: 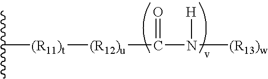
wherein R₁₁, R₁₂ and R₁₁₃ are each independently selected from the group consisting of optionally substituted alkyl, optionally substituted alkenyl, optionally substituted alkynyl, optionally substituted cycloalkyl, optionally substituted aryl, optionally substituted heteroaryl, or optionally substituted heterocycloalkyl;
t, u, v and w are each independently 0 or 1, provided that at least one of t, u and w is 1;
R₇ is a group of formula:
   (R₁₆)_{z}—(R₁₅)_{y}—(R₁₄)ₓ—
wherein R₁₄, R₁₅ and R₁₆ are independently selected from the group consisting of optionally substituted alkyl, optionally substituted alkenyl, optionally substituted alkynyl, optionally substituted cycloalkyl, optionally substituted aryl, optionally substituted heteroaryl and optionally substituted heterocycloalkyl,
x, y and z are independently 0 and 1 with the proviso that at least one of x, y and z is 1.

In one particular embodiment of the invention the compound having the formula (I) is based on cysteine. Accordingly, the embodiment of the invention provides a compound of formula (IIa), or a pharmaceutically acceptable derivative, salt, racemate, isomer or tautomer thereof: 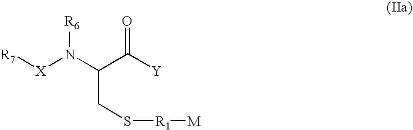
wherein R₁, R₆, R₇, M, X and Y are as defined above for the compound of formula (I).

In another embodiment of the invention the compound having the formula (I) is based on a 7-substituted 2-amino-heptanoates. Accordingly, the embodiment of the invention provides a compound of formula (IIb), or a pharmaceutically acceptable derivative, salt, racemate, isomer or tautomer thereof: 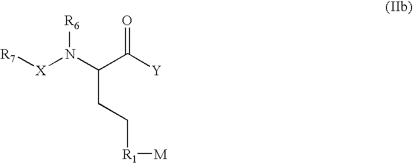
wherein R₁, R₆, R₇, M, X and Y are as defined above for the compound of formula (I).

As would be clear to a skilled addressee any number of suitable moieties can be used as the linking moiety of the compounds of the invention. It is typical, however, that the linking moiety is a hydrocarbyl moiety that is unbranched. Moieties of this type are the simplest to produce and are found to not interfere with the activity of the remainder of the compound. It is preferred that the linker has between 1 and 9 atoms in the normal chain, preferably between 1 and 4 atoms in the normal chain.

In addition the zinc binding moiety can be chosen so that it is any suitable moiety that will bind to zinc. There are a number of suitable zinc binding moieties well known in the art. Examples of well known zinc binding moieties include sulfur donors (such as HS—R, wherein R is defined above), amine containing compounds (primary, secondary, tertiary amines), heterocyclic amines, carboxylates, amino acids, thiolates, dithiocarbamates, phosphorodidithiolates and the like. Some examples of suitable moieties within these subsets are as follows:

Sulfur donors (thioproline, penicillamine, cysteine, 2-mercaptoethylamine, glutathione, methionine, thiosulfate, N-acetylcysteine, penicillaminedisulfide, thiomalate, and 2,3-dimercaptosuccinate

Aliphatic amines (histamine, trien, Me4en)

Heterocyclic amines (pipicolate, nicotinate, picolinate, 8-hydroxyquinoline, bicinchoninate, bipy, phendisulfonate)

Carboxylates (acetate, propionate, tartrate, succinate, malate, gluconate, betahydroxybutyrate, lactate, salicylate, citrate, ascorbate, oxalate, EDTA)

Amino acids (gly, arg, asn, glu, asp, glygly, glyglygly, glyglyhis, pro, 2,3-diaminopropionate, 2-amino-2-deoxygluconate, his)

It is preferred that the zinc binding ligand is a hydroxamic acid derivative.

As with all chemical families there are a number of preferred embodiments within the scope of the general formula.

It is preferred, for example that the group Y is a group of formula —NR₄R₅.

It is particularly preferred that the zinc binding moiety containing a heteroatom is a hydroxamic acid derivative, preferably a group of formula —C(O)—NR₂—OR₃ where R₂ is H, optionally substituted alkyl, optionally substituted alkenyl, optionally substituted alkynyl, optionally substituted aryl, or a nitrogen protecting group and R₃ is H, optionally substituted alkyl, optionally substituted alkenyl, optionally substituted alkynyl, optionally substituted aryl or an oxygen protecting group;

Accordingly in a preferred embodiment the present invention provides a compound having the formula (III), or a pharmaceutically acceptable derivative, salt, racemate, isomer or tautomer thereof: 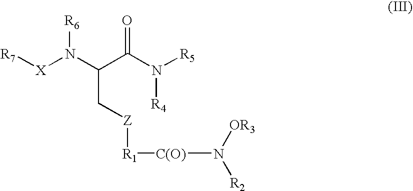
wherein
Z is S or CH₂;
R₁ is optionally substituted C₁-C₄ alkyl, optionally substituted C₁-C₄ alkenyl or optionally substituted C₁-C₄ alkynyl;
R₂ is H, optionally substituted alkyl, optionally substituted alkenyl, optionally substituted alkynyl, optionally substituted aryl, or a nitrogen protecting group;
R₃ is H, optionally substituted alkyl, optionally substituted alkenyl, optionally substituted alkynyl, optionally substituted aryl or an oxygen protecting group;
R₄ is a group of formula: 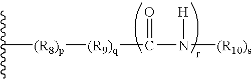
wherein R₈, R₉ and R₁₀ are each independently selected from the group consisting of optionally substituted alkyl, optionally substituted alkenyl, optionally substituted alkynyl, optionally substituted cycloalkyl, optionally substituted aryl, optionally substituted heteroaryl, and optionally substituted heterocycloalkyl;
p, q, r and s are each independently 0 or 1, provided that at least one of p, q or s is 1;
R₅ is H or a group of formula: 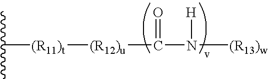
wherein R₁₁, R₁₂ and R₁₁₃ are each independently selected from the group consisting of optionally substituted alkyl, optionally substituted alkenyl, optionally substituted alkynyl, optionally substituted cycloalkyl, optionally substituted aryl, optionally substituted heteroaryl, or optionally substituted heterocycloalkyl;
t, u, v and w are each independently 0 or 1, provided that at least one of t, u and w is 1.
R₆ is selected from the group consisting of H, optionally substituted alkyl, optionally substituted alkenyl, optionally substituted alkynyl and a nitrogen protecting group;
X is selected from the group consisting of 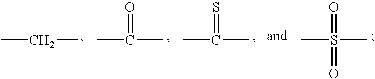
R₇ is a group of formula:
   (R₁₆)_{z}—(R₁₅)_{y}—(R₁₄)ₓ—
wherein R₁₄, R₁₅ and R₁₆ are independently selected from the group consisting of optionally substituted alkyl, optionally substituted alkenyl, optionally substituted alkynyl, optionally substituted cycloalkyl, optionally substituted aryl, optionally substituted heteroaryl and optionally substituted heterocycloalkyl;
x, y and z are independently 0 and 1 with the proviso that at least one of x, y and z is 1.

As stated previously there are a number of compounds within the scope of the general structural formula that are preferred. There are therefore a number of preferred variables for each of the substituents in the general formula. For example it is preferred that R₁ is optionally substituted C₁-C₄ alkyl, more preferably optionally substituted C₂-C₃ alkyl, even more preferably optionally substituted C₃ alkyl, most preferably propyl.

It is preferred that R₂ is either H, optionally substituted C₁-C₄ alkyl or a nitrogen protecting group, more preferably H or a nitrogen protecting group, most preferably H.

It is preferred that R₃ is either H, optionally substituted C₁-C₄ alkyl or an oxygen protecting group, more preferably H or an oxygen protecting group, most preferably H.

In a most preferred embodiment, the compounds are of formula (IIIa) and (IIIb).

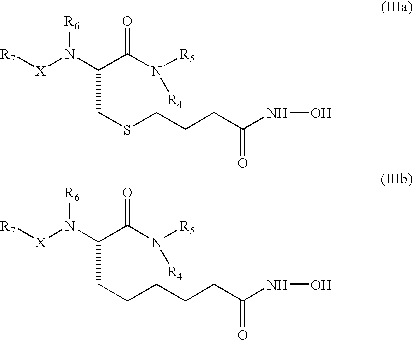

In the compounds of the invention it is particularly preferred that X is a carbonyl group.

It is preferred that R₅ is H.

It is preferred that R₆ is either H or a nitrogen protecting group, most preferably H.

In one preferred embodiment the group R₄ is of the formula 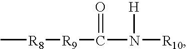
wherein R₈, R₉ and R₁₀ are as defined above.

In this embodiment it is particularly preferred that R₄ is of the formula:

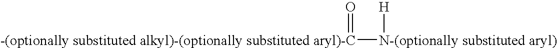

In the most preferred form of this embodiment R₄ is a group of the formula. 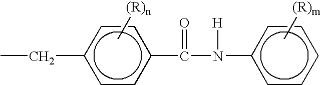
wherein each R is independently selected from the group consisting of alkyl, alkenyl, alkynyl, aryl, heteroaryl, cycloalkyl, heterocycloalkyl, halo, haloalkyl, haloalkenyl, haloalkynyl, haloaryl, haloheteroaryl, halocycloalkyl, haloheterocycloalkyl, hydroxy, alkoxy, alkenyloxy, aryloxy, heteroaryloxy, cycloalkyloxy, heterocycloalkyloxy, benzyloxy, haloalkoxy, haloalkenyloxy, haloaryloxy, halohetoraryloxy, nitro, nitroalkyl, nitroalkenyl, nitroalkynyl, nitroaryl, nitroheteroaryl, nitroheterocyclyoalkyl, amino, alkylamino, dialkylamino, alkenylamino, alkynylamino, arylamino, heteroarylamino, diarylamino, benzylamino, dibenzylamino, acyl, alkenylacyl, alkynylacyl, arylacyl, heteroarylacyl, acylamino, diacylamino, acyloxy, alklysulphonlyoxy, arylsulphonyloxy, heterocycloalkylamino, alkylsulphonyl, arylsulphonyl, carboalkoxy, carboaryloxy, alkylthio, benzylthio, acylthio, cyano, nitro, sulfate and phosphate;
n is 0-4, and
m is 0-5.

In an another preferred embodiment of the invention R₄ is selected from the group consisting of: optionally substituted alkyl, optionally substituted alkenyl, optionally substituted alkynyl, optionally substituted aryl, optionally substituted cycloalkyl, optionally substituted heteroaryl, optionally substituted heterocycloalkyl, optionally substituted arylalkyl, optionally substituted heteroarylalkyl, optionally substituted cycloalkylalkyl, optionally substituted heterocycloalkylalkyl, optionally substituted aryl alkenyl, optionally substituted heteroaryl alkenyl, optionally substituted cycloalkyl alkenyl, optionally substituted heterocycloalkyl alkenyl, optionally substituted aryl alkynyl; optionally substituted heteroaryl alkynyl optionally substituted cycloalkyl alkynyl, optionally substituted heterocycloalkyl alkynyl.

In this embodiment it is particularly preferred that R₄ is selected from optionally substituted aryl, optionally substituted cycloalkyl, optionally substituted heteroaryl, optionally substituted alkyl, optionally substituted arylalkyl, optionally substituted heteroarylalkyl, optionally substituted cycloalkyl alkyl, optionally substituted alkyl aryl, optionally substituted alkyl heteroaryl, optionally substituted alkyl heterocycloalkyl.

In a most preferred embodiment of the invention R₄ has one of the following formulae. 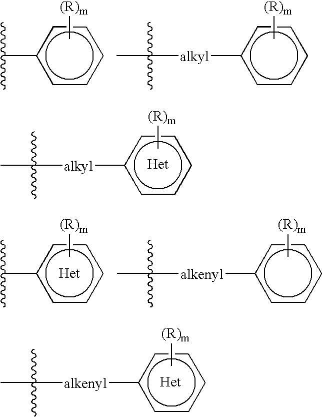
wherein each R is independently selected from the group consisting of alkyl, alkenyl, alkynyl, aryl, heteroaryl, cycloalkyl, heterocycloalkyl, halo, haloalkyl, haloalkenyl, haloalkynyl, haloaryl, haloheteroaryl, halocycloalkyl, haloheterocycloalkyl, hydroxy, alkoxy, alkenyloxy, aryloxy, heteroaryloxy, cycloalkyloxy, heterocycloalkyloxy, benzyloxy, haloalkoxy, haloalkenyloxy, haloaryloxy, halohetoraryloxy, nitro, nitroalkyl, nitroalkenyl, nitroalkynyl, nitroaryl, nitroheteroaryl, nitroheterocyclyoalkyl, amino, alkylamino, dialkylamino, alkenylamino, alkynylamino, arylamino, heteroarylamino, diarylamino, benzylamino, dibenzylamino, acyl, alkenylacyl, alkynylacyl, arylacyl, heteroarylacyl, acylamino, diacylamino, acyloxy, alklysulphonlyoxy, arylsulphonyloxy, heterocycloalkylamino, alkylsulphonyl, arylsulphonyl, carboalkoxy, carboaryloxy, alkylthio, benzylthio, acylthio, cyano, nitro, sulfate and phosphate;
and each m is from 0-5.

Preferred values of R as substituents on R₄ are dialkyl amino, acyl, aryl, carboalkoxy, benzyl, cycloalkyl, heteroaryl, hydroxy, halo and cyano. Particularly preferred values of R₄ are dimethyl amino, diethyl amino, bromo, phenyl and benzyl.

In the compounds of the invention it is preferred that R₇ is selected from the group consisting of optionally substituted alkyl, optionally substituted alkenyl, optionally substituted alkynyl, optionally substituted aryl, optionally substituted cycloalkyl, optionally substituted heteroaryl, optionally substituted heterocycloalkyl, optionally substituted aryl alkyl, optionally substituted heteroaryl alkyl, optionally substituted cycloalkyl alkyl, optionally substituted heterocycloalkyl alkyl, optionally substituted aryl alkenyl, optionally substituted hetero alkenyl, optionally substituted cycloalkyl alkenyl, optionally substituted heterocycloalkyl alkenyl, optionally substituted aryl alkynyl, optionally substituted heteroaryl alkynyl, optionally substituted cycloalkyl alkynyl, optionally substituted and heterocycloalkyl alkynyl.

It is even more preferred that R₇ is optionally substituted aryl, optionally substituted heteroaryl, optionally substituted alkyl, optionally substituted cycloalkyl, optionally substituted heterocycloalkyl, optionally substituted aryl alkyl, optionally substituted alkenyl, optionally substituted aryl alkenyl.

It is most preferred that R₇ has one of the following formula: 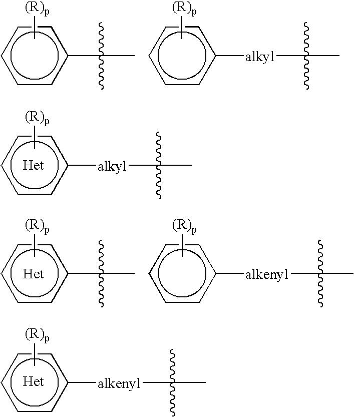
wherein each R is independently related from the group consisting of alkyl, alkenyl, alkynyl, aryl, heteroaryl, cycloalkyl, heterocycloalkyl, halo, haloalkyl, haloalkenyl, haloalkynyl, haloaryl, haloheteroaryl, halocycloalkyl, haloheterocycloalkyl, hydroxy, alkoxy, alkenyloxy, aryloxy, heteroaryloxy, cycloalkyloxy, heterocycloalkyloxy, benzyloxy, haloalkoxy, haloalkenyloxy, haloaryloxy, halohetoraryloxy, nitro, nitroalkyl, nitroalkenyl, nitroalkynyl, nitroaryl, nitroheteroaryl, nitroheterocyclyoalkyl, amino, alkylamino, dialkylamino, alkenylamino, alkynylamino, arylamino, heteroarylamino, diarylamino, benzylamino, dibenzylamino, acyl, alkenylacyl, alkynylacyl, arylacyl, heteroarylacyl, acylamino, diacylamino, acyloxy, alklysulphonlyoxy, arylsulphonyloxy, heterocycloalkylamino, alkylsulphonyl, arylsulphonyl, carboalkoxy, carboaryloxy, alkylthio, benzylthio, acylthio, cyano, nitro, sulfate and phosphate;
and each p is from 0-5.

Particularly preferred values of R as a substituent on an R₇ group are dialkylamino, alkoxy, halo, aryl, alkyl, hydroxy, nitro and arylamino.

Preferred compounds of the invention include those listed in tables 1 to 9 in the examples.

Synthesis of the Compounds of the Invention

Compounds of formula (I) may be generated in a number of ways depending on the synthetic strategy adopted and the available starting materials. As would be clear to a skilled addressee the exact method utilised will depend on the available starting materials.

Isolation and purification of the compounds and intermediates described herein can be effected, if desired, by any suitable separation or purification procedure such as, for example, filtration, extraction, crystallisation, column chromatography, thin-layer chromatography, thick-layer (preparative) chromatography, distillation, HPLC or a combination of these procedures. Specific illustrations of suitable separation and isolation procedures can be had by reference to the examples provided herein. However, other equivalent separation or isolation procedures can also be used.

Preparation of Compounds of Formula (IIa)

The applicants have identified an efficient methodology for producing the compounds of formula (IIa) that progresses through an advanced intermediate of formula (3): 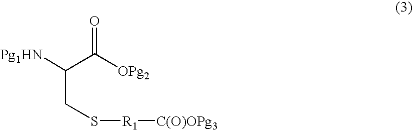
Wherein Pg₁ is a protecting group for nitrogen and Pg₂ and Pg₃ are protecting groups for oxygen and R₁ is as previously defined. The protecting groups in formula (3) may be any suitable groups that are suitably adapted for the remaining steps of the process. It is important, however that the two carboxylic acid protecting groups can be differentially de-protected so that the two groups can be separately functionalised. A preferred form of the compound of formula (3) can be made utilising the reaction sequence outlined in scheme 1. Modifications to this general scheme can be made to produce compounds of formula (3) with other protecting groups and/or general structures. The extent of the modifications and the way in which could be done are well within the ambit of a skilled addressee in the art. 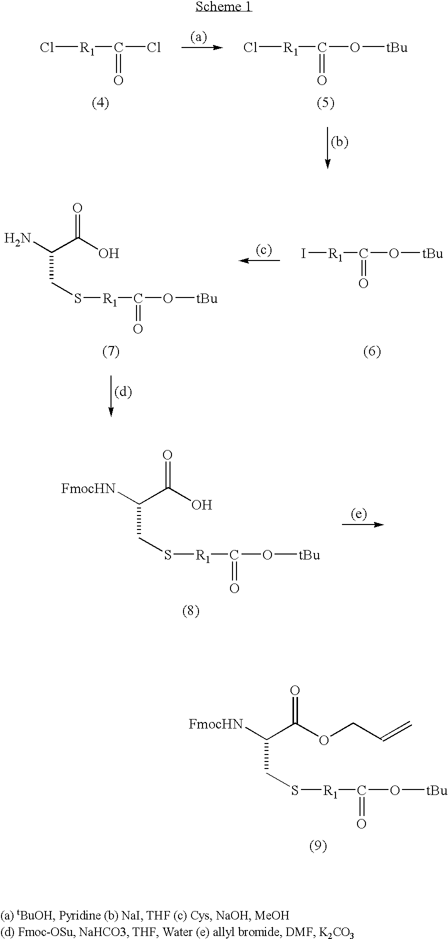
Referring to scheme 1 carboxylic acid chloride (4) with the desired R₁ group is converted to the protected form by reaction with tertiary butanol in pyridine to produce the t-butyl protected form (5). The choice of protecting group will vary depending on a number of factors including the identity of the further protecting group chosen. The choice of a suitable protecting group will typically not cause difficulty for a skilled addressee and can vary greatly with the preferred group being t-butyl.

The protected carboxylic acid (5) is then reacted with sodium iodide to produce the iodinated derivative (6). This is then reacted with an appropriate thio derivative such as cysteine to produce intermediate (7). This compound is then protected at both the C and N termini. Accordingly it is preferred that the compound is reacted with a nitrogen protecting group such as Fmoc to produce the N-protected compound (8) which is then in turn reacted with allyl bromide to produce the final differentially protected compound (9). In the preferred embodiment of the invention R₁ is propyl and the production of the preferred compounds follows an analogous procedure as that shown in scheme 1 with the starting compound (4) being the acid chloride of 4-chlorobutyric acid. In order to vary the group R₁ in the final compounds of the invention all that is required is that the starting material (4) contain the suitable R₁. In general a skilled worker in the field will easily be able to produce a wide range of compounds of general formula (4) with different values of R₁ from commercially available starting materials. In addition whilst in the reaction scheme shown above the iodinated compound (6) is reacted with naturally occurring cysteine it could equally be reacted with the unnatural isomer or even a mixture of isomers.

The compounds of formula (9) are then converted into compounds of the invention utilising the general procedure given in scheme 2.

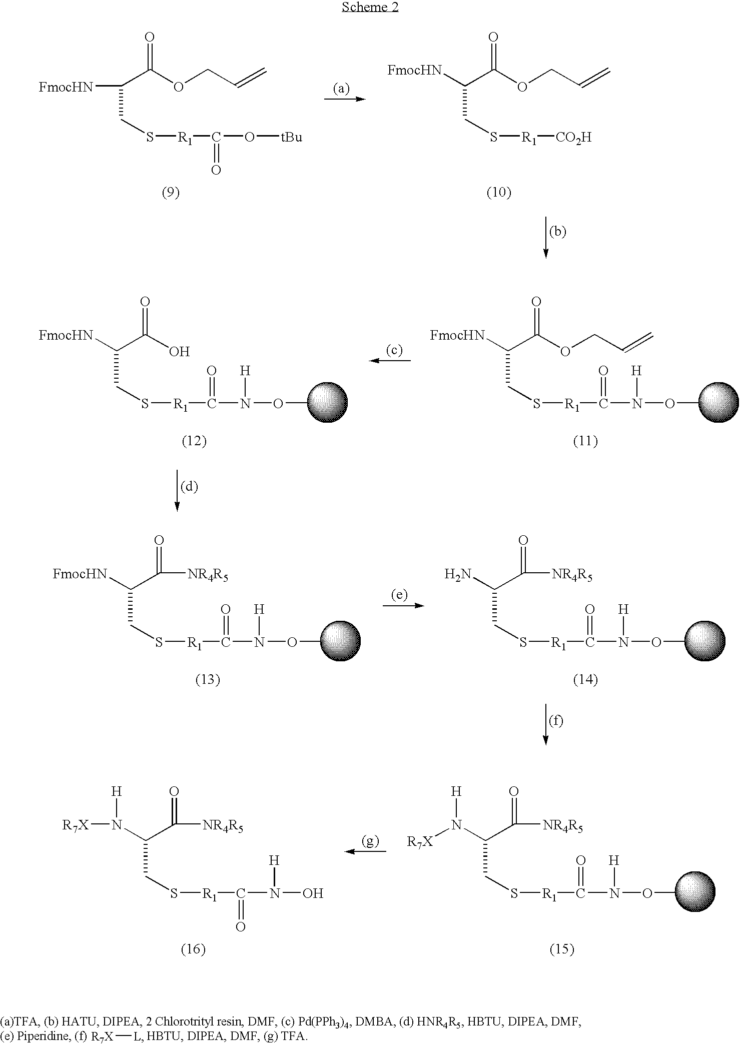

Thus the compound of formula (9) is de-protected by reaction with TFA to differentially remove the t-butyl protecting group and form compound (10). This de-protected compound is then reacted with an appropriately modified resin to immobilise the compound on the resin and form immobilised compound (11). The immobilised compound is then treated with palladium to remove the allyl protecting group to form immobilised acid (12). Reaction of acid (12) with an appropriately substituted nucleophillic compound such as amine of formula (HNR₄R₅) produces advanced compound (13). This is then reacted with piperidine to remove the Fmoc protecting group to produce the free amine (14). Reaction of amine (14) with a group of formula R₇XL where L is a leaving group then produces compound (15). The compound can then be removed from the solid support by reaction with TFA under appropriate conditions to form the compound (16) of the invention.

Preparation of Compounds of Formula (IIb)

Using a similar methodology to that described for compounds of formula (IIa), synthesis of the compounds of formula (IIb) progresses through an advanced intermediate of formula (17):

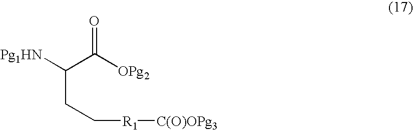

Again, Pg₁ is a protecting group for nitrogen and Pg₂ and Pg₃ are protecting groups for oxygen and R₁ is as previously defined. The protecting groups in formula (17) may be any suitable groups that are suitably adapted for the remaining steps of the process. A preferred form of the compound of formula (17) can be made utilising the reaction sequence outlined in scheme 3. Modifications to this general scheme can be made to produce compounds of formula (17) with other protecting groups and/or general structures. The extent of the modifications and the way in which could be done are well within the ambit of a skilled addressee in the art.

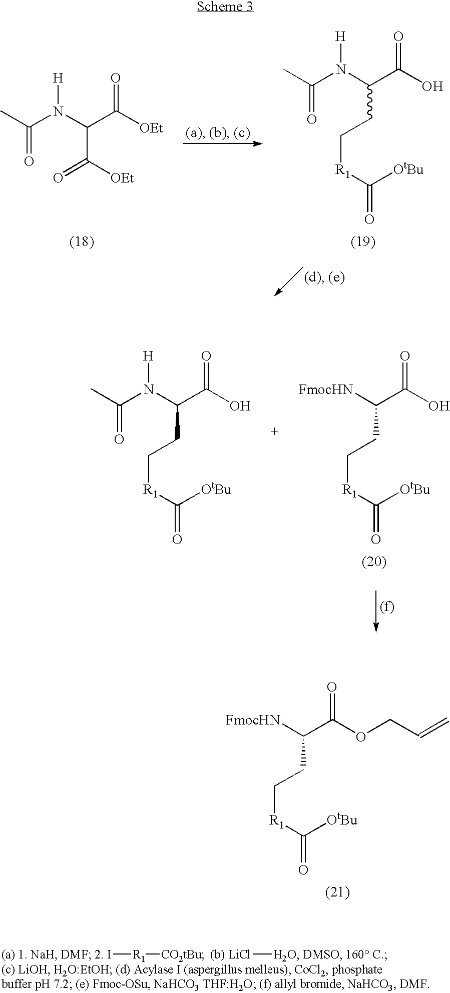

Referring to scheme 3, malonate diester (18) is alkylated with the desired R₁ carboxylate having an appropriate leaving group (e.g. iodide) and then decarboxylated and saponified to produce the acid (19). Enzymatic resolution followed by protection with a nitrogen protecting group such as Fmoc produces the N-protected (S)-enantiomer (20) which is then in turn reacted with allyl bromide to produce the final differentially protected intermediate (21).

In a preferred embodiment of the invention R₁ is propyl and the production of the preferred compounds follows an analogous procedure as that shown in scheme 3 with the malonate diester (18) being reacted with 6-iodohexanoic acid tert-butyl ester. In order to vary the group R₁ in the final compounds of the invention all that is required is that the iodo acid tert-butyl ester contains the suitable R₁. In general a skilled worker in the field will easily be able to produce a wide range of suitable compounds with different values of R₁ from commercially available starting materials. In addition whilst in the reaction scheme shown above the racemate (19) is resolved by enzymatic resolution, it will be appreciated that the chiral resolution may be omitted and the acetate group removed from the N-terminus in the racemic mixture and the free amine then protected with a suitable N-protecting group (in this case Fmoc) to produce a racemic mixture of compound (20) which can then be carried through the remainder of the steps.

The compounds of formula (21) are then converted into the compounds of the invention utilising the general procedure given in scheme 4. 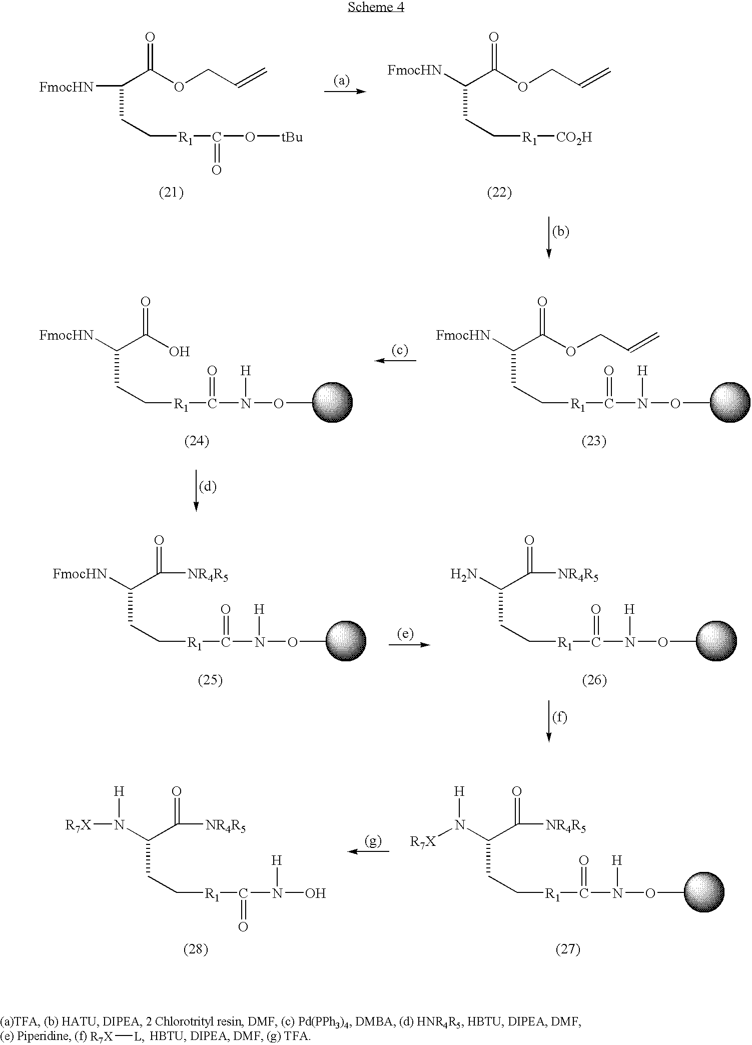
It will be appreciated the steps of scheme 4 may be carried out in the same manner as the steps of scheme 2.
Use of Compounds of the Invention for the Treatment of Cancer

The present invention also provides a method for the treatment of cancer in an animal, the method including the step of administering to the animal in need of such treatment an effective amount of a compound having the formula (I), as hereinbefore described, or a pharmaceutically acceptable derivative, salt, racemate, or isomer thereof.

The compounds of this invention may be administered in compositions such as tablets, capsules or elixirs for oral administration, suppositories, sterile solutions or suspensions for injectable administration, and the like, or incorporated into shaped articles. Typical adjuvants which may be incorporated into tablets, capsules and the like are a binder such as acacia, corn starch or gelatin, and excipient such as microcrystalline cellulose, a disintegrating agent like corn starch or alginic acid, a lubricant such as magnesium stearate, a sweetening agent such as sucrose or lactose, or a flavoring agent. When a dosage form is a capsule, in addition to the above materials it may also contain a liquid carrier such as water, saline, fatty oil. Other materials of various types may be used as coatings or as modifiers of the physical form of the dosage unit. Sterile compositions for injection can be formulated according to conventional pharmaceutical practice. For example, dissolution or suspension of the active compound in a vehicle such as an oil or a synthetic fatty vehicle like ethyl oleate, or into a liposome may be desired. Buffers, preservatives, antioxidants and the like can be incorporated according to accepted pharmaceutical practice.

While the preferred route of administration is oral, other methods of administration are also anticipated such as intravenously (bolus and/or infusion), subcutaneously, intramuscularly, transdermally, colonically, rectally, nasally or intraperitoneally, employing a variety of dosage forms such as suppositories, implanted pellets or small cylinders, aerosols, injectable formulations and topical formulations such as ointments, drops and dermal patches. The compounds of this invention could be incorporated into shaped articles such as implants which may employ inert materials such as biodegradable polymers or synthetic silicones, for example, Silastic, silicone rubber or other polymers commercially available.

The compounds of this invention may also be administered in the form of liposome delivery systems, such as small unilamellar vesicles, large unilamellar vesicles and multilamellar vesicles. Liposomes can be formed from a variety of lipids, such as cholesterol, stearylamine or phosphatidylcholines.

Formulations of the compounds of this invention are prepared for storage or administration by mixing the compound having a desired degree of purity with physiologically acceptable carriers, excipients, stabilisers etc., and may be provided in sustained release or timed release formulations. Acceptable carriers or diluents for therapeutic use are well known in the pharmaceutical field, and are described, for example, in Remington's Pharmaceutical Sciences, Mack Publishing Co., (A. R. Gennaro edit. 1985). Such materials are nontoxic to the recipients at the dosages and concentrations employed, and may include buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinalpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counterions such as sodium and/or nonionic surfactants such as Tween, Pluronics or polyethyleneglycol.

Animals in need of treatment using the compounds of this invention can be administered dosages that will provide optimal efficacy. The dose and method of administration will vary from animal to animal and be dependent upon such factors as the type of mammal being treated, its sex, weight, diet, concurrent medication, overall clinical condition, the particular compounds employed, the specific use for which these compounds are employed, and other factors which those skilled in the medical arts will recognise.

Therapeutically effective dosages may be determined by either in vitro or in vivo methods. For each particular compound of the present invention, individual determinations may be made to determine the optimal dosage required. The range of therapeutically effective dosages will naturally be influenced by the route of administration, the therapeutic objectives, and the condition of the patient. It may be necessary for the therapist to titer the dosage and modify the route of administration as required to obtain the optimal therapeutic effect. The determination of effective dosage levels, that is, the dosage levels necessary to achieve the desired result, will be within the knowledge of one skilled in the art. For example it is typical that for any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of the PK activity). Such information can then be used to more accurately determine useful doses in humans.

Toxicity and therapeutic efficacy of the compounds described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition—(See e.g., Fingl, et al., 1975, in “The Pharmacological Basis of Therapeutics”, Ch. 1 p. 1). Typically, applications of compound are commenced at lower dosage levels, with dosage levels being increased until the desired effect is achieved.

Generally a dosage of as little as about 1-2 milligram (mg) per kilogram (kg) of body weight is suitable, but preferably as little as 1 mg/kg and up to about 100 mg/kg may be used. Preferably, a dosage from 2 mg/kg to about 40 mg/kg is used. Most preferably, the dose is between 4 mg/kg to about 8 mg/kg. Any range of doses can be used. Generally, a compound, salt thereof, prodrug thereof, or combination of the present invention can be administered on a daily basis one or more times a day, or one to four times a week, either in a single dose or separate doses during the day. Twice-weekly dosing over a period of at least several weeks is preferred, and often dosing will be continued over extended periods of time and possibly for the lifetime of the patient. However, the dosage and the dosage regimen will vary depending on the ability of the patient to sustain the desired and effective plasma levels of the compounds of the present invention, or salt or prodrug thereof, in the blood.

In practicing the methods of this invention, the compounds of this invention may be used alone or in combination, or in combination with other therapeutic or diagnostic agents. In certain preferred embodiments, the compounds of this invention may be co-administered along with other compounds typically prescribed for these conditions according to generally accepted medical practice. For example, compounds of this invention may be used in combination with DNA methyltransferase inhibitors (as described in Herman J G and Baylin S B (2003) NEJM 349, 2042-2054). Such inhibitors may include but are not limited to 5-azacytidine, deoxy-5-azacytidine, or zebularine.

The compounds of this invention may also be delivered by the use of antibodies, antibody fragments, growth factors, hormones, or other targeting moieties, to which the compound molecules are coupled. The compounds of this invention may also be coupled with suitable polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the compounds of this invention may be coupled to a biodegradable polymer for achieving controlled release of a drug. Examples of such polymers include polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like.

Without further description, it is believed that one of ordinary skill in the art can, using the preceding description and the following illustrative examples, make and utilise the compounds of the present invention and practice the claimed methods.

The following abbreviations are used in the examples and elsewhere throughout the specification:

Ac=acetyl;

DCM=Dichloromethane;

DIPEA=diisopropylethylamine;

DMAP=4-(Dimethylamino)pyridine;

DMBA=1,3-Dimethylbarbituric acid;

DMF=dimethylformamide;

EtOAc=Ethyl acetate;

Fmoc-OSu=9-Fluorenylmethyloxycarbonyl-N-hydroxysuccinimide;

HATU=O-(7-azabenzotriazol-1-yl)-1,1,3,3tetramethyluroniumhexafluoro phosphate;

HBTU=[(benzotriazolyl)oxy]-N′,N′,N′,N′-tatramethyluroniumhexafluorophosphate; rpHPLC=reverse phase high performance liquid chromatography;

LRMS=Low resolution mass spectroscopy;

TFA=trifluoroacetic acid;

THF=tetrahydrofuran.

The following working examples therefore, specifically point out preferred embodiments of the present invention, and are not to be construed as limiting in any way the remainder of the disclosure.

### EXAMPLES OF PREFERRED EMBODIMENTS OF THE INVENTION

### General methods

¹H NMR spectra were recorded on either a Bruker ARX 500 MHz or a Varian 300 MHz NMR spectrometer. Semi preparative scale rpHPLC separations were performed on a Phenomenex Luna 5μ C18(2) 250×21 mm column run at 20 mL/minute using gradient mixtures of water/0.1% TFA (A) and water (10%)/acetonitrile (90%)/0.1% TFA (B), and product fractions were always lyophilized to dryness. Preparative scale rpHPLC separations were performed on a Vydac 218TP101550 50×250 mm column run at 70 mL/minute using gradient mixtures of A and B. Accurate mass determinations were performed on an API QSTAR mass spectrometer using electron impact ionization. Water octanol partition coefficients (Log D) were calculated using PALLAS prolog D 2.1. Molecular modeling was performed on an SGI Octane R12000, with minimization calculation performed with the cff91 force field using the Discover Module within Insight II.

### Example 1

### Coupling of Acid to Resin

### General Method

Commercially available N-Fmoc hydroxylamine 2-chlorotrityl resin (0.77 mmol/g, 10 g, 7.7 mmol) was shaken gently with 1:1 piperidine:DMF (30 mL) over night, and then flow washed with DMF for 1 minute. In a separate flask, HATU (3.0 g, 7.8 mmol) was added to a solution of the acid (7.8 mmol) and DIPEA (5.3 mL, 31.2 mmol) dissolved in DMF (10 mL), and the resulting solution stirred gently for 5 minutes. The HATU activated acid was then added in one portion to the deprotected resin, and the resin was shaken gently for 1 hour. After washing the resin well with DMF, the resin loading was determined. The unreacted resin was then acylated by addition of a solution of acetic anhydride (842 mg, 7.8 mmol) and DIPEA (5.3 mL, 31.2 mmol) in DMF (20 mL) with shaking for 2 minutes, followed by thorough washing with DMF.

### Example 2

### Coupling of Acid Moiety with Functional Group to Add NR₄R₅ Group

### General Method

The resin (0.45 mmol/g, 200 mg, 0.09 mmol) was shaken in DMF (1 mL) for 10 minutes, and then DIPEA (122 μL, 0.72 mmol) and 0.5 M HBTU in DMF (360 μL, 0.18 mmol) were introduced and shaking continued for a further 5 minutes. The amine (0.25 mmol) was then added, and shaking continued for a further 1 hour. After washing the resin well with DMF, cleavage of a small portion of resin and analysis by mass spectroscopy generally indicates 60-85% conversion to the amide.

### Example 3

### Coupling of Amine Moiety with Functional Group to Add R₇X Group

### General Method

The resin was shaken in DMF (1 mL) for 10 minutes, the DMF removed, and then 1:1 piperidine:DMF (1 mL) added. After shaking for 5 minutes the piperidine:DMF was removed, and the resin washed well with DMF. This procedure was repeated two more times. In a separate flask 0.5 M HBTU (180 μL, 90 μmol) in DMF was added to a solution of the desired acid (90 μM) and DIPEA (76 μL, 450 μmol) in DMF (1 mL), and the resulting solution stirred for 5 minutes before being added in one portion to the resin. The resin was shaken for 1 hour, and then washed well with DMF. Cleavage of a small portion of resin and analysis by mass spectroscopy generally indicates 100% conversion to the amide.

### Example 4

### Cleavage of Immobilised Compound from Resin

### General Method

The resin was washed well with DCM, and then drained. TFA:water (99:1, 1 mL) was added, and the resin shaken for 20 minutes. The TFA was collected, and the resin washed with a further 1 mL of TFA. The TFA was removed by distillation. Purification was performed by rpHPLC, and hydroxamates confirmed to be greater than 95% pure by analytical rpHPLC and ¹H NMR spectroscopy.

Production of a Preferred Intermediates

### Example 5

### 4-Chloro-butyric acid tert-butyl ester

4-Chlorobutyryl chloride (16.6 mL, 147 mmol) was added drop wise to a cooled (0° C.) solution of DMAP (10 mg) in equal portions of tert-butanol (50 mL) and pyridine (50 mL). After complete addition of the acid chloride, the resulting suspension was stirred for 1 hour, and then solvent removed under reduced pressure. The residue was dissolved in EtOAc (500 mL), and washed successively with saturated NaHCO₃ and NaCl solutions. The organic layer was dried over magnesium sulfate, and solvent removed to provide the tert-butyl ester as a clear oil (22.3 g, 85%). ¹H NMR (CDCl₃, 300 MHz): 3.58 (t (6.4 Hz), 2H); 2.40 (t (7.3 Hz), 2H); 2.06 (m, 2H); 1.45 ppm (s, 9H). ¹³C NMR (CDCl₃, 75 MHz): 172.5, 81.1, 44.8, 33.1, 28.6, 28.4 ppm.

### Example 6

### 4-Iodo-butyric acid tert-butyl ester

Sodium iodide (70.0 g, 467 mmol) was added to tert-butyl ester of example 5 (22.0 g, 124 mmol) dissolved in THF (300 mL), and the resulting yellow suspension was refluxed overnight. The solvent was removed under reduced pressure, and the residue dissolved in EtOAc (200 mL). After washing successively with water and saturated NaCl solution the organic phase was dried over magnesium sulfate, and solvent removed to provide the title iodide as a yellow oil (31.4 g, 94%). ¹H NMR (CDCl₃, 300 MHz): 3.23 (t (6.7 Hz), 2H); 2.34 (t (7.3 Hz), 2H); 2.07 (m, 2H); 1.40 ppm (s, 9H). ¹³C NMR (CDCl₃, 75 MHz): 173.2, 81.0, 44.6, 35.1, 28.9, 6.0 ppm.

### Example 7

### 4-((2S)-Amino-2-carboxy-ethylsulfanyl)-butyric acid tert-butyl ester

A suspension of cysteine (6.6 g, 55.5 mmol) in methanol (50 mL) was cooled to 0° C. and degassed under a stream of argon for 5 minutes. On addition of 2M sodium hydroxide solution (55.5 mL, 111 mmol) the cysteine dissolved, and tert-butyl ester of example 6 (15.0 g, 55.5 mmol) was added immediately in one portion. Stirring was continued for a further 5 minutes, before adjustment of the pH to ˜8 with 2 M HCl. The solvent was removed under reduced pressure, and the residue desalted by rpHPLC to provide the title amino acid as a white solid (14.2 g, 97%). ¹H NMR (d₆-DMSO, 300 MHz): 3.4 to 3.1 (br s, water); 2.97 (dd (3.8, 14.3 Hz), 1H); 2.70 (dd (8.7, 14.2 Hz), 1H); 2.51 (t (7.4 Hz), 2H); 2.29 (t (7.2 Hz), 2H); 1.72 (m, 2H); 1.38 ppm (s, 9H). ¹³C NMR (d₆DMSO, 75 MHz): 175.2, 172.6, 80.2, 53.9, 34.0, 33.2, 30.6, 28.1, 24.7 ppm.

### Example 8

### 4-[(2S)-Carboxy-2-(9H-fluoren-9-ylmethoxycarbonylamino)-ethylsulfanyl]-butyric acid tert-butyl ester

NaHCO₃ (14 g, 170 mmol) and Fmoc-OSu (18.7 g, 55.5 mmol) were added to a solution of amino acid of example 7 (14.0 g, 53.0 mmol) dissolved in 1:1 THF water (300 mL), and the resulting solution stirred for 2 hours. The solvent was removed under reduced pressure, and the residue suspended in EtOAc (300 mL), and washed successively with water, 1 M HCl, saturated NaHCO₃ solution and brine. The organic layer was dried over magnesium sulfate, and solvent removed under reduced pressure to yield a yellow oil which was purified by rpHPLC to provide the title acid as a white solid (19.7 g, 76%). ¹H NMR (d₆-DMSO, 300 MHz): 7.90 (d (7.5 Hz), 2H); 7.73 (d (7.71 Hz), 2H); 7.42 (t (7.2 Hz), 2H); 7.32 (t (6.6 Hz), 2H); 4.65 (d (5.3 Hz), 2H); 4.30 (m, 2H); 2.91 (dd (3.7, 14.2 Hz), 1H); 2.76 (dd (8.6, 14.2 Hz), 1H); 2.51 (t (7.3 Hz), 2H); 2.26 (t (7.2 Hz), 2H); 1.72 (m, 2H); 1.39 ppm (s, 9H). ¹³C NMR (d₆-DMSO, 75 MHz): 174.6, 172.1, 156.2, 144.2, 144.1, 128.0, 127.4, 125.6, 120.5, 79.6, 60.5, 54.5, 46.8, 34.0, 33.2, 30.8, 28.1, 24.8 ppm.

### Example 9

### 4-[(2S)-Allyloxycarbonyl-2-(9H-fluoren-9-ylmethoxycarbonylamino)-ethylsulfanyl]-butyric acid tert-butyl ester

Allyl bromide (6.23 g, 51.5 mmol) was added in one portion to a suspension of K₂CO₃ (27 g, 200 mmol) and compound of example 8 (25.0 g, 51.5 mmol) in DMF (200 mL). The resulting solution stirred for 10 minutes, and then the solvent was removed under reduced pressure. The resulting residue was dissolved in EtOAc (500 mL) and washed successively with water, 1 M HCl, saturated NaHCO₃ solution, and brine. The organic layer was dried over magnesium sulfate, and solvent removed under reduced pressure to provide the title allyl ester as a yellow oil (25.0 g, 92%). ¹H NMR (d₆-DMSO, 300 MHz): 7.89 (d (7.60 Hz), 2H); 7.72 (d (7.71 Hz), 2H); 7.41 (t (7.1 Hz), 2H); 7.32 (t (7.3 Hz), 2H); 5.88 (m, 1H); 5.31 (d (16.7 Hz), 1H); 5.20 (d (11.7 Hz), 1H); 4.59 (d (5.3 Hz), 2H); 4.25 (m, 4H); 2.88 (dd (3.8, 14.1 Hz), 1H); 2.77 (m, 1H); 2.53 (t (7.3 Hz), 2H); 2.27 (t (7.3 Hz), 2H); 1.72 (m, 2H); 1.38 ppm (s, 9H). ¹³C NMR (d₆-DMSO, 75 MHz): 172.1, 170.9. 156.3, 144.1, 141.1, 132.6, 128.0, 127.4, 125.6, 120.5, 118.1, 80.0, 66.2, 60.1, 54.5, 47.0, 34.0, 32.7, 31.0, 28.1, 24.9 ppm.

### Example 10

### 4-[2-Allyloxycarbonyl-2-(9H-fluoren-9-ylmethoxycarbonylamino)-ethylsulfanyl]-butyric acid

The ester of example 9 (25.0 g, 47.5 mmol) was stirred in 99:1 TFA:water (50 mL) for 2 hours. The solvent was removed under reduced pressure, and the residue purified by rpHPLC to provide the title acid as a white solid (19.5 g, 88%). ¹H NMR (d₆-DMSO, 300 MHz): 7.89 (d (7.1 Hz), 2H); 7.72 (d (7.1 Hz), 2H); 7.42 (t (7.1 Hz), 2H); 7.33 (t (7.6 Hz), 2H); 5.90 (m, 1H); 5.30 (d (17.3 Hz), 1H); 5.19 (d (9.4 Hz), 1H); 4.59 (d (5.2 Hz), 2H); 4.28 (m, 4H); 2.89 (dd (4.9, 13.5 Hz), 1H); 2.79 (m, 1H); 2.52 (t (7.3 Hz), 2H); 2.29 (t (7.3 Hz), 2H); 1.73 ppm (m, 2H). ¹³C NMR (d₆-DMSO, 75 MHz): 174.4, 170.9, 156.3, 144.1, 141.1, 132.6, 128.0, 127.4, 125.6, 120.5, 118.4, 66.2, 60.1, 54.5, 47.0, 32.8, 32.7, 31.1, 24.7 ppm.

### Example 11

### Coupling to Acid of Example 10 to Resin

Commercially available N-Fmoc hydroxylamine 2-chlorotrityl resin (0.77 mmol/g, 10 g, 7.7 mmol) was shaken gently with 1:1 piperidine:DMF (30 mL) over night, and then flow washed with DMF for 1 minute. In a separate flask HATU (3.0 g, 7.8 mmol) was added to a solution of acid of example 10 (3.7 g, 7.8 mmol) and DIPEA (5.3 mL, 31.2 mmol) dissolved in DMF (10 mL), and the resulting solution stirred gently for 5 minutes. The HATU activated acid was then added in one portion to the deprotected resin, and the resin was shaken gently for 1 hour. After washing the resin well with DMF, the resin loading was determined to be 0.46 mmol/g (70%) (LRMS m/e calc. for C₂₅H₂₉N₂O₆S (MH⁺) 485.6, obs. 485.1). The unreacted resin was then acylated by addition of a solution of acetic anhydride (842 mg, 7.8 mmol) and DIPEA (5.3 mL, 31.2 mmol) in DMF (20 mL) with shaking for 2 minutes, followed by thorough washing with DMF.

### Example 12

### Removal of the Allyl Protecting Group

The resin of example 11 was flow washed with DCM for 2 minutes, and then shaken in DCM (30 mL) for a further 10 minutes. An argon stream was introduced, and the resin and DCM degassed for 5 minutes. DMBA (1.2 g, 7.9 mmol) was added, and bubbling continued for a further minute to ensure thorough mixing. Pd(PPh₃)₄ (270 mg, 0.23 mmol) was added to the resin, the flask wrapped in aluminum foil, and after a further 30 seconds of degassing the argon stream was removed, and the resin shaken gently for 1 hour. The resin was flow washed successively with DCM, DMF, and DCM, before drying under high vacuum. The resin loading was determined to be 0.45 mmol/g (LRMS m/e calc. for C₂₂H₂₅N₂O₆S (MH⁺) 445.5, obs. 445.2).

### Example 13

6-Iodo-hexanoic acid tert-butyl ester: 6-Bromo-hexanoic acid (10 g, 51.3 mmol) was dissolved in 1,4-dioxane (30 mL) in a pressure vessel and cooled in a dry-ice bath (acetone). Isobutylene (30 mL) was added to the solution followed by H₂SO₄ (0.5 mL). The vessel was closed and the mixture was stirred at RT for 48 hrs before it was poured into a separatory funnel with sat. NaHCO₃ (aq) (150 mL), extracted with diethyl ether (3×150 mL) and washed with brine (2×150 mL). The organic phase was dried (MgSO₄) and evaporated and further dissolved in THF (200 mL). NaI (30.7 g, 205 mmol) was added to the reaction flask and the mixture was refluxed for 16 hrs. When the reaction mixture had cooled to RT, diethyl ether was added to the solution which made most of the salt precipitate. The salt was filtered off with a sintered glad funnel and the solvent was poured into a separatory funnel, extracted with diethyl ether (3×200 mL) and washed with brine (2×200 mL). The organic phase was dried (MgSO4), evaporated and purified by chromatography (petroleum ether: ethyl acetate, 9:1) to give a yellow oil in 90% yield over two steps. ¹H NMR (CDCl3, 600 MHz): 1.40-1.43 (m, 4H), 1.43 (s, 9H), 1.56-1.62 (m, 2H), 1.79-1.85 (m, 2H), 2.21 (t, 2H, J=7.5 Hz), 3.18 (t, 2H, J=7.0 Hz). ¹³C NMR (CDCl3, 125 MHz): 7.1, 24.1, 28.3, 28.3, 28.3, 30.0, 33.3, 35.4, 80.3, 173.1.

### Example 14

2-Acetylamino-2-ethoxycarbonyl-octanedioic acid 8-tert-butyl ester 1-ethyl ester: NaH (60% dispersion in mineral oil) (3.97 g, 99.1 mmol) was added to a solution of diethyl acetamidomalonate (19.57 g, 90.1 mmol) dissolved in DMF (150 mL). After 30 min Iodo-hexanoic acid tert-butyl ester (30 g, 117.2 mmol) was added to the mixture and the solution was stirred at RT for 4 hrs. The reaction mixture was poured into a separatory funnel, extracted with (3×150 mL) diethyl ether and washed with brine (2×150 mL). The organic phase was dried (MgSO₄), evaporated and purified by chromatography (petroleum ether: ethyl acetate, 3:1) to give a yellow oil in 93% yield. ¹H NMR (CDCl3, 600 MHz): 1.08-1.13 (m, 2H); 1.24 (t, 6H, J=7.1 Hz), 1.28-1.33 (m, 2H), 1.42 (s, 9H), 1.52-1.57 (m, 2H), 2.03 (s, 3H), 2.16 (t, 2H, J=7.2 Hz), 2.29-2.32 (m, 2H), 4.23 (q, 4H, J=7.1 Hz), 6.77 (bs, 1H).). ¹³C NMR (CDCl3, 125 MHz): 14.2, 23.2, 23.6, 25.1, 28.3, 28.9, 32.2, 35.5, 60.6, 62.7, 66.7, 80.2, 168.4, 169.1, 173.2.

### Example 15

2-Acetylamino-octanedioic acid 8-tert-butyl ester 1-ethyl ester: LiCl.H2O (622 mg, 14.5 mmol) and H2O (347 μL, 19.3 mmol) was added to a solution of 2-Acetylamino-2-ethoxycarbonyl-octanedioic acid 8-tert-butyl ester 1-ethyl (3.736 g, 9.64 mmol) dissolved in DMSO (50 mL). The mixture was heated to 150 oC for 16 hrs then extracted with diethyl ether (3×100 mL) and washed with brine (2×100 mL). The organic phase was dried (MgSO4), evaporated and put on high vacuum for 10 hrs to give the product in 98% yield as a yellow oil. ¹H NMR (CDCl3, 600 MHz): 1.27 (t, 3H, J=7.2 Hz), 1.28-1.33 (m, 2H), 1.42 (s, 9H), 1.53-1.58 (m, 2H), 1.62-1.67 (m, 2H), 1.79-1.84 (m, 2H), 2.01 (s, 3H), 2.18 (t, 2H, J=7.6 Hz), 4.19 (q, 4H, J=7.3 Hz), 4.55-4.59 (m, 1H), 6.08 (d, 1H, J=7.7 Hz). ¹³C NMR (CDCl3, 125 MHz): 14.3, 23.4, 25.0, 25.0, 28.3, 28.3, 28.8, 32.6, 35.5, 52.3, 61.6, 80.2, 170.0, 172.9, 173.2.

### Example 16

2-Acetylamino-octanedioic acid 8-tert-butyl ester: LiOH.H2O (1.79 g, 42.5 mmol) was added to 2-Acetylamino-octanedioic acid 8-tert-butyl ester 1-ethyl ester (8.93 g, 28.4 mmol) dissolved in 100 mL of H2O:EtOH (1:1). The pH was made neutral by citric acid (aq) after ca 1 hr and the EtOH was removed by evaporation. The solution was the poured into a separatory funnel, extracted with EtOAc (3×150 mL) and washed with brine (2×150 mL). The organic phase was dried (MgSO4), evaporated and purified by chromatography (petroleum ether: ethyl acetate, 1:1) to give a pale yellow oil in 93% yield. ¹H NMR (CDCl3, 600 MHz): 1.30-1.38 (m, 2H), 1.45 (s, 9H), 1.55-1.62 (m, 2H), 1.64-1.76 (m, 3H), 1.87-1.93 (m, 1H), 2.06 (s, 3H), 2.22 (t, 2H, J=7.4 Hz), 4.56-4.61 (m, 1H), 6.30 (d, 1H, J=7.3 Hz). ¹³C NMR (CDCl3, 151 MHz): 23.0, 24.7, 24.8, 28.1, 28.5, 31.7, 35.3, 52.3, 80.3, 170.9, 173.4, 175.3.

### Example 17

2-(9H-Fluoren-9-ylmethoxycarbonylamino)-octanedioic acid 8-tert-butyl ester: 2-Acetylamino-octanedioic acid 8-tert-butyl ester (7.5 g, 26.2 mmol) was dissolved in phosphate buffer (0.1 M, pH 7.2, 500 mL), and the pH adjusted to 7.2 by addition of 2M NaOH. The resulting solution was warmed to 39° C., and CoCl2.6H2O (75 mg) was added with gentle shaking. Acylase I (aspergillus melleus, 375 mg) was added to the solution, and the reaction was left to sit 48 hrs at 39° C. Analysis of an aliquot of the solution by ¹H NMR indicated a 1:1 mixture of the amine and the acetamide. The solvent was removed to about half the volume by evaporation and 250 mL of THF was added. NaHCO3 (4.4 g, 52.4 mmol) and FMOC-Succinate (4.6, 13.7 mmol) was added to the solution and the mixture was stirred for 2 hours. The solvent was removed under reduced pressure, and the residue suspended in EtOAc (300 mL), and washed successively with water, 1 M HCl, saturated NaHCO3 solution and brine. The organic layer was dried (MgSO4), evaporated and purified by chromatography (petroleum ether: ethyl acetate, 2:1) to give a pale yellow oil in 45% yield. ¹H NMR (CDCl3, 600 MHz): 1.35-1.44 (m, 4H), 1.45 (s, 9H), 1.59-1.62 (m, 2H), 1.69-1.73 (m, 1H), 1.88-1.92 (m, 1H), 2.22 (t, 2H, J=7.4 Hz), 3.92 (bs, 1H), 4.23 (t, 1H, J=7.0 Hz), 4.38-4.45 (m, 3H), 5.33 (d, 1H, J=8.2 Hz), 7.32 (dd, 2H, J=7.4, 7.3 Hz), 7.41 (dd, 2H, J=7.4 Hz), 7.61 (m, 2H), 7.77 (d, 2H, J=7.5 Hz). ¹³C NMR (CDCl3, 151 MHz): 24.9, 25.1, 28.3, 28.7, 32.3, 35.6, 47.4, 53.8, 67.3, 80.5, 120.2, 125.3, 127.4, 128.0, 141.5, 143.9, 156.3, 173.6, 176.4.

### Example 18

2-(9H-Fluoren-9-ylmethoxycarbonylamino)-octanedioic acid 1-allyl ester 8-tert-butyl ester: Allyl bromide (1.74 g, 14.4 mmol) was added in one portion to a suspension of NaHCO3 (4.4 g, 52.4 mmol)) and the ester from example 17 (5.5 g, 11.8 mmol) in DMF (200 mL). The resulting solution stirred for 30 minutes, and then the solvent was removed under reduced pressure. The resulting residue was dissolved in EtOAc (500 mL) and washed successively with water and brine (2×200 mL). The organic layer was dried (MgSO4), and solvent removed under reduced pressure to provide the title allyl ester as a yellow oil in 92% yield. ¹H NMR (CDCl3, 600 MHz): 1.31-1.40 (m, 4H), 1.45 (s, 9H), 1.56-1.62 (m, 2H), 1.67-1.73 (m, 1H), 1.85-1.90 (m, 1H), 2.21 (t, 2H, J=7.5 Hz), 4.24 (t, 1H, J=7.1 Hz), 4.40-4.42 (m, 3H), 4.66 (bs, 2H), 5.27-5.37 (m, 2H), 5.91-5.93 (m, 1H), 7.33 (dd, 2H, J=7.5 Hz), 7.41 (dd, 2H, J=7.5 Hz), 7.60-7.62 (m, 2H), 7.78 (d, 2H, J=7.6 Hz). ¹³C NMR (CDCl3, 151 MHz): 25.0, 25.1, 28.3, 28.8, 32.7, 35.6, 47.4, 54.1, 66.2, 67.2, 80.3, 119.2, 120.2, 125.9, 127.3, 127.9, 131.7, 141.5, 144.0, 144.1, 156.1, 172.5, 173.2.

### Example 19

2-(9H-Fluoren-9-ylmethoxycarbonylamino)-octanedioic acid 1-allyl ester: Tert-butyl ester from example 18 (4.0 g, 7.83 mmol) was stirred in 9:1 TFA:DCM (50 mL) for 30 min. The solvent was removed under reduced pressure, and the residue purified by flash chromatography (petroleum ether: ethyl acetate, 2:1) to provide the title acid as a white solid in 89% yield. ¹H NMR (CDCl3, 600 MHz): 1.26=7−1.42 (m, 4H), 1.63-1.70 (m, 3H), 1.86-1.88 (m, 1H), 2.35 (t, 2H, J=7.4 Hz), 4.24 (t, 1H, J=7.0 Hz), 4.41-4.42 (m, 3H), 4.66 (bs, 2H), 5.27-5.37 (m, 2H), 5.91-5.93 (m, 1H), 7.33 (dd, 2H, J=7.4 Hz), 7.41 (dd, 2H, J=7.5 Hz), 7.60-7.62 (m, 2H), 7.78 (d, 2H, J=7.6 Hz). ¹³C NMR (DMSO, 125 MHz): 24.3, 24.8, 28.5, 32.4, 33.7, 47.1, 53.8, 65.9, 67.0, 118.9, 119.9, 125.0, 127.0, 127.7, 131.4, 141.3, 143.7, 143.8, 155.9, 172.3, 179.1.

### Example 20

Coupling of acid to resin: Commercially available N-Fmoc hydroxylamine 2-chlorotrityl resin (0.77 mmol/g, 7.54 g, 5.81 mmol) was shaken gently with 1:1 piperidine:DMF (20 mL) over night, and then washed through with DMF 10 times. In a separate flask, HATU (2.26 g, 6.10 mmol) was added to a solution of 2-(9H-Fluoren-9-ylmethoxycarbonylamino)-octanedioic acid 1-allyl ester (3.14 g, 7.0 mmol) and DIPEA (5.06 mL, 29.0 mmol) dissolved in DMF (10 mL), and the resulting solution stirred gently for 10 minutes. The HATU activated acid was then added in one portion to the deprotected resin, and the resin was shaken gently for 1 hour. After washing the resin well with DMF, the resin loading was determined to be 0.522 mmol/g (91%) (LRMS m/e calc. for C26H30N2O6 (MH+) 467.2, obs. 467.2). The unreacted resin was then acylated by addition of a solution of acetic anhydride (842 mg, 7.8 mmol) and DIPEA (5.3 mL, 31.2 mmol) in DMF (20 mL) with shaking for 2 minutes, followed by thorough washing with DMF.

### Example 21

Removal of the allyl ester: The resin was flow washed with DCM for 2 minutes, and then shaken in DCM (30 mL) for a further 10 minutes. A nitrogen stream was introduced, and the resin and DCM degassed for 5 minutes. DMBA (0.80 g, 5.12 mmol) was added, and bubbling continued for a further minute to ensure thorough mixing. Pd(Ph3)4 (493 mg, 0.43 mmol) was added to the resin, the flask wrapped in aluminum foil, and after a further 30 seconds of degassing the nitrogen stream was removed, and the resin shaken gently for 1 hour. The resin was flow washed successively with DCM, DMF, and DCM, before drying under high vacuum. (LRMS m/e calc. for C23H26N2O6 (MH+) 427.2, obs. 427.1).

The products of examples 12 and 21 were subjected to the general procedures outlined in examples 2-4 with variations made to the amine moiety used for coupling to the acid in example 2 and the acid moiety used for reaction in example 3 to produce the compounds given in the following tables. For example utilising benzylamine as the amine used according to the procedure in example 2 and by using a number of acids as the coupling moiety according to the general procedure of example 3 the compounds in table 1 were produced as examples 22-58. Similarly, by utilising 4-dimethylamino benzoic acid as the coupling moiety according to the general procedure of example 3 and varying the amine used according to the procedure in example 2 the compounds in table 2 were produced as examples 59-96.

Following similar methodology using cinnamic acid as the coupling moiety according to the general procedure of example 3 and varying the amine used according to the procedure in example 2 the compounds in table 3 were produced as examples 97-102.

Similarly, by utilising 4-dimethylamino benzoic acid as the coupling moiety according to the general procedure of example 3 and varying the amine used according to the procedure in example 2 the compounds in table 8 of examples 103-121 were produced.

Likewise, by using the 7-substituted 2-amino-heptanoate (21) and varying the acid as the coupling moiety according to the general procedure of example 3, or by varying the amine used according to the procedure of example 2 the compounds in table 4 of examples 122 to 168 were produced.

TABLE 1
HPLC Retention Time and HRMS Data for Compounds of Examples 22-
57

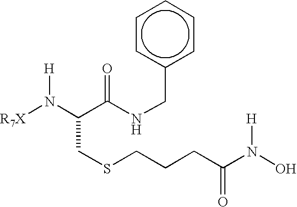

| Compound | | RpHPLC | RT-grad | HRMS | MS- |
|---|---|---|---|---|---|
| of Example | R₇—X | RT-Iso (min) | (min) | (g/mol) | theoretical |
| 22 | 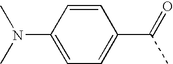 | 9.17 | 17.04 | 459.2051 | 459.2061 |
| 23 | 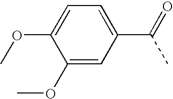 | 2.32 | 16.66 | 445.1909 | 445.1904 |
| 24 | 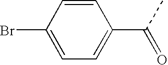 | 7.25 | 24.56 | 494.0742 | 494.0744 |
| 25 | 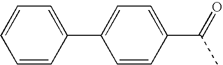 | 12.00 | 26.57 | 492.1987 | 492.1987 |
| 26 | 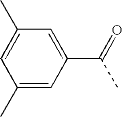 | 7.45 | 24.69 | 444.1989 | 444.1952 |
| 27 | 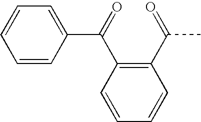 | 11.46 | 26.27 | 521.1745 | 521.1741 |
| 28 | 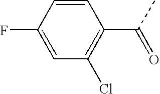 | 11.41 | 18.91 | 468.1143 | 468.1155 |
| 29 | 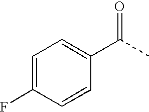 | 10.87 | 18.61 | 434.1528 | 434.1545 |
| 30 | 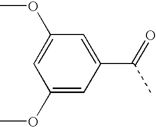 | 11.60 | 10.97 | 476.1867 | 476.1850 |
| 31 | 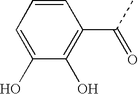 | 9.39 | 17.34 | 448.1538 | 448.1537 |
| 32 | 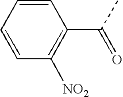 | 10.15 | 17.96 | 461.1504 | 461.1490 |
| 33 | 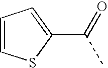 | 4.48 | 21.77 | 422.1211 | 422.1203 |
| 34 | 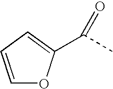 | 9.25 | 17.00 | 406.1419 | 406.1431 |
| 35 | 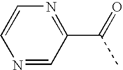 | 3.44 | 19.79 | 418.1574 | 418.1544 |
| 36 | 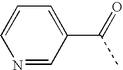 | 7.80 | 14.60 | 417.1593 | 417.1591 |
| 37 | 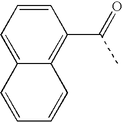 | 7.87 | 24.99 | 466.1816 | 466.1795 |
| 38 | 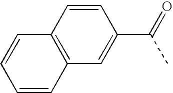 | 7.53 | 24.77 | 466.1791 | 466.1795 |
| 39 | 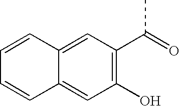 | 20.93 | 21.72 | 482.1759 | 482.1744 |
| 40 | 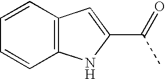 | 6.26 | 23.94 | 455.1744 | 455.1748 |
| 41 | 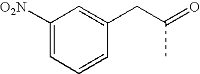 | 10.69 | 18.42 | 475.1672 | 475.1646 |
| 42 | 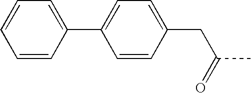 | 11.92 | 26.49 | 506.2143 | 506.2108 |
| 43 | 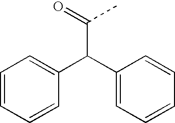 | 17.66 | 21.09 | 506.2098 | 506.2108 |
| 44 | 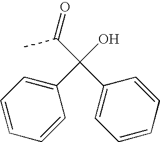 | 8.81 | 25.38 | 522.2088 | 522.2057 |
| 45 | 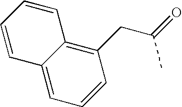 | 7.60 | 24.78 | 480.1955 | 480.1952 |
| 46 | 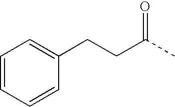 | 11.46 | 18.94 | 444.1950 | 444.1952 |
| 47 | 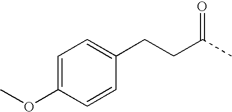 | 11.21 | 18.83 | 474.2058 | 474.2057 |
| 48 | 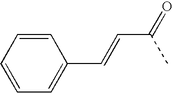 | 5.95 | 23.67 | 442.1830 | 442.1795 |
| 49 | 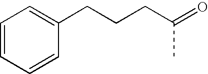 | 7.13 | 24.49 | 458.2127 | 458.2108 |
| 50 | 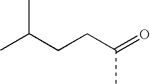 | 5.53 | 22.92 | 410.2127 | 410.2108 |
| 51 | 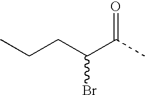 | 5.46 | 23.01 | 422.2131 | 422.2108 |
| 52 | 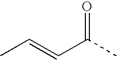 | 8.89 | 16.60 | 380.1638 | 380.1639 |
| 53 | 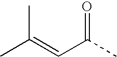 | 9.72 | 17.54 | 394.1790 | 394.1795 |
| 54 | 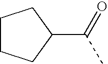 | 10.19 | 17.96 | 408.1949 | 408.1952 |
| 55 | 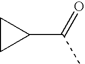 | 8.81 | 16.38 | 380.1640 | 380.1639 |
| 56 | 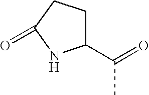 | 7.72 | 24.85 | 423.1701 | 423.1697 |
| 57 | 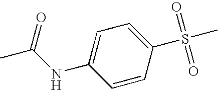 | 0.6 | 17.51 | 434.1388 | 434.1381 |

TABLE 2
HPLC Retention Time and HRMS Data for Compounds of Examples
59-96

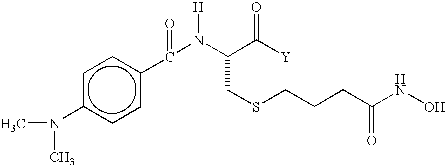

| Compound | | RT-Iso | RT-grad | HRMS | MS- |
|---|---|---|---|---|---|
| of Example | Y | (min) | (min) | (g/mol) | theoretical |
| 59 | 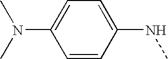 | 7.66 | 15.36 | 488.2301 | 488.2326 |
| 60 | 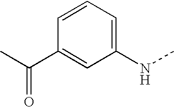 | 8.30 | 18.23 | 487.2025 | 487.2010 |
| 61 | 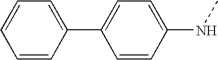 | 8.83 | 25.38 | 521.2199 | 521.2217 |
| 62 | 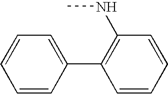 | 9.75 | 21.61 | 521.2237 | 521.2217 |
| 63 | 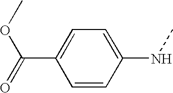 | 9.98 | 20.16 | 503.1969 | 503.1959 |
| 64 | 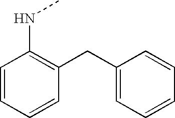 | 12.01 | 20.05 | 535.2393 | 535.2374 |
| 65 | 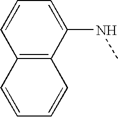 | 10.56 | 18.97 | 495.2058 | 495.2061 |
| 66 | 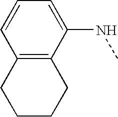 | 10.87 | 19.75 | 499.2396 | 499.2374 |
| 67 | 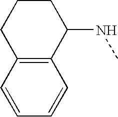 | 9.25 | 20.55 | 499.2396 | 499.2374 |
| 68 | 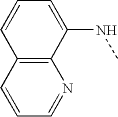 | 9.77 | 17.95 | 496.2005 | 496.2013 |
| 69 | 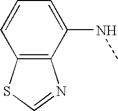 | 11.10 | 19.18 | 502.1590 | 502.1577 |
| 70 | 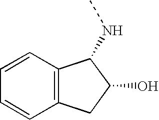 | 8.85 | 19.98 | 501.2148 | 501.2166 |
| 71 | 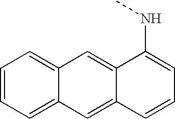 | 10.88 | 23.21 | 545.2228 | 545.2217 |
| 72 | 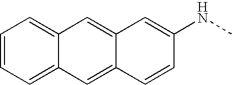 | 12.64 | 24.48 | 545.2228 | 545.2217 |
| 73 | 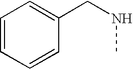 | 9.15 | 20.57 | 459.2074 | 459.2061 |
| 74 | 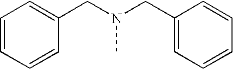 | 10.27 | 22.44 | 549.2547 | 549.2530 |
| 75 | 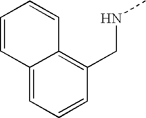 | 11.06 | 23.10 | 509.2230 | 509.2217 |
| 76 | 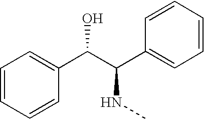 | 9.71 | 21.45 | 565.2452 | 565.2479 |
| 77 | 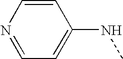 | 7.56 | 15.14 | 446.1773 | 446.1791 |
| 78 | 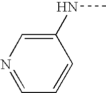 | 7.76 | 16.03 | 446.1776 | 446.1791 |
| 79 | 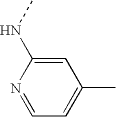 | 7.67 | 15.65 | 460.2009 | 460.2013 |
| 80 | 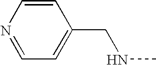 | 7.38 | 14.34 | 460.2009 | 460.2013 |
| 81 | 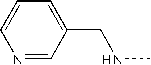 | 8.01 | 17.79 | 460.2009 | 460.2013 |
| 82 | 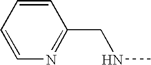 | 7.46 | 14.58 | 460.2009 | 460.2013 |
| 83 | 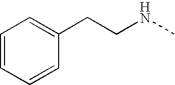 | 10.07 | 18.31 | 473.2227 | 473.2217 |
| 84 | 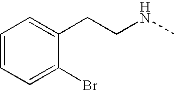 | 8.76 | 19.55 | 551.1312 | 551.1322 |
| 85 | 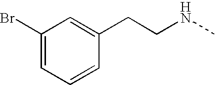 | 8.98 | 20.06 | 551.1315 | 551.1322 |
| 86 | 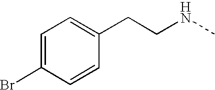 | 11.28 | 23.26 | 551.1305 | 551.1322 |
| 87 | 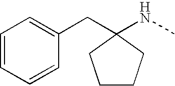 | 10.26 | 22.37 | 527.2697 | 527.2687 |
| 88 | 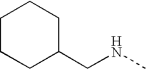 | 8.91 | 19.92 | 465.2521 | 465.2530 |
| 89 | 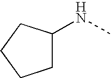 | 9.40 | 17.12 | 437.2206 | 437.2217 |
| 90 | 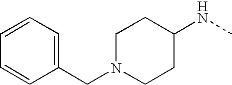 | 8.13 | 17.76 | 542.2802 | 542.2796 |
| 91 | 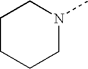 | 7.95 | 16.92 | 437.2199 | 437.2217 |
| 92 | 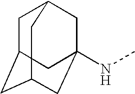 | 9.79 | 21.65 | 503.2699 | 503.1697 |
| 93 | 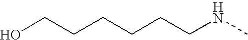 | 8.34 | 15.38 | 455.2344 | 455.2323 |
| 94 | 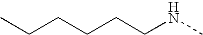 | 8.52 | 16.72 | 439.2347 | 439.2374 |
| 95 | 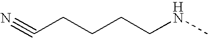 | 7.88 | 16.54 | 464.2339 | 464.2321 |
| 96 | 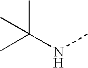 | 8.87 | 19.50 | 425.2194 | 425.2217 |

TABLE 3
HPLC Retention Time and HRMS Data for Compounds of Examples
97-102

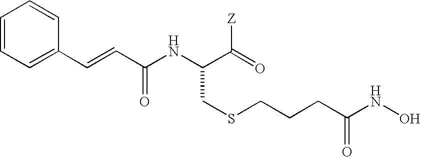

| Compound | | RT-Iso | RT-grad | | MS- |
|---|---|---|---|---|---|
| of Example | Z | (min) | (min) | HRMS (g/mol) | theoretical |
| 97 | 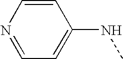 | 8.21 | 18.84 | 429.1610 | 429.1591 |
| 98 | 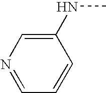 | 7.95 | 17.10 | 429.1610 | 429.1591 |
| 99 | 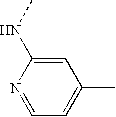 | 8.10 | 18.02 | 443.1610 | 443.1591 |
| 100 | 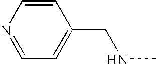 | 7.82 | 17.63 | 443.1611 | 443.1591 |
| 101 | 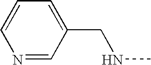 | 7.73 | 15.47 | 443.1610 | 443.1591 |
| 102 | 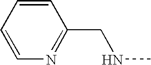 | 8.03 | 18.09 | 443.1612 | 443.1591 |

TABLE 4
HPLC Retention Time and HRMS Data for Selected Compounds of
Examples 122-168

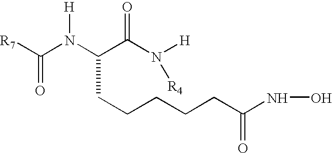

| | | | RT- | RT- | | |
|---|---|---|---|---|---|---|
| Compound | | | Iso | Grad | HRMS | MS- |
| of Example | R₇ | R₄ | (min) | (min) | (g/mol) | theoretical |
| 122 | 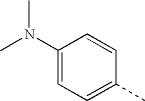 | 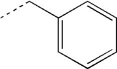 | | | | 441.2496 |
| 123 | 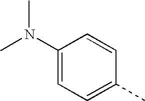 | 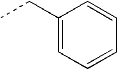 | | | | 441.2496 |
| 124 | 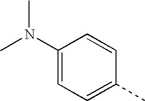 | 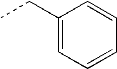 | | | | 441.2496 |
| 125 | 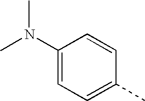 | 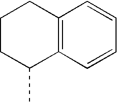 | | | | 481.2809 |
| 126 | 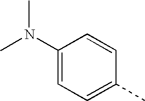 | 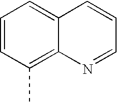 | 3.88 | 20.23 | 478.2456 | 478.2449 |
| 127 | 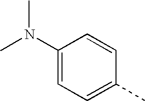 | 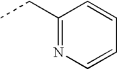 | | | | 442.2449 |
| 128 | 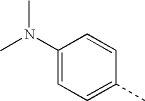 | 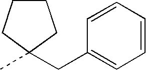 | | | | 509.3122 |
| 129 | 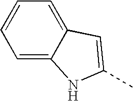 | 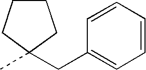 | | | | 505.2809 |
| 130 | 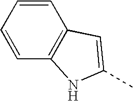 | 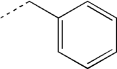 | 3.61 | 21 | 437.2206 | 437.2183 |
| 131 | 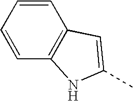 | 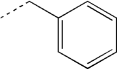 | | | 437.2206 | 437.2183 |
| 132 | 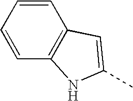 | 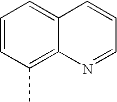 | 6.81 | 23.10 | 474.2148 | 474.2136 |
| 133 | 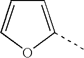 | 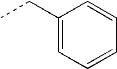 | | | | 388.1867 |
| 134 | 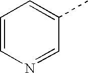 | 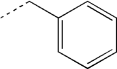 | | | | 399.2027 |
| 135 | 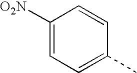 | 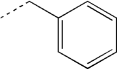 | | | | 443.1925 |
| 136 | 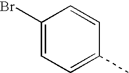 | 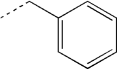 | 3.77 | 21.26 | 476.1200 | 476.1179 |
| 137 | 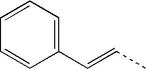 | 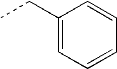 | 3.55 | 20.52 | 424.2215 | 424.2231 |
| 138 | 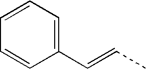 | 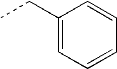 | | | | 424.2231 |
| 139 | 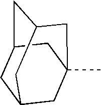 | 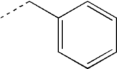 | 4.78 | 22.88 | 456.2842 | 456.2857 |
| 140 | 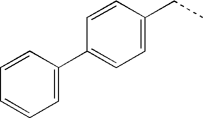 | 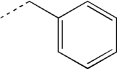 | 4.94 | 23.20 | 488.2527 | 488.2544 |
| 141 | 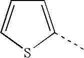 | 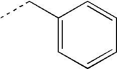 | 3.11 | 18.66 | 404.1641 | 404.1639 |
| 142 | 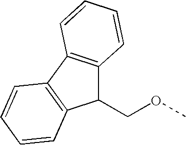 | 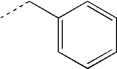 | 6.55 | 24.50 | 516.2501 | 516.2493 |
| 143 | 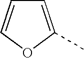 | 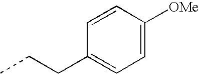 | | | | 416.2180 |
| 144 | 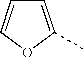 | 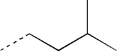 | | | | 368.2180 |
| 145 | 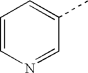 | 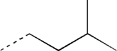 | | | | 379.2340 |
| 146 | 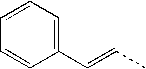 | 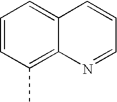 | 6.04 | 22.57 | 461.2174 | 461.2184 |
| 147 | 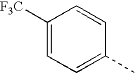 | 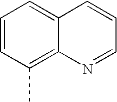 | 12.23 | 25.20 | 503.1899 | 503.1901 |
| 148 | 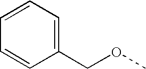 | 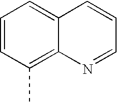 | 8.61 | 23.87 | 465.2137 | 465.2133 |
| 149 | 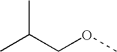 | 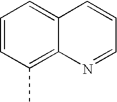 | 7.67 | 23.43 | 432.2299 | 431.2289 |
| 150 | 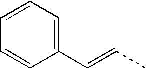 | 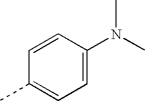 | 3.09 | 19.55 | 453.2496 | 453.2497 |
| 151 | 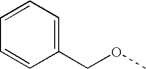 | 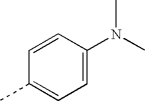 | 3.17 | 18.46 | 457.2425 | 457.2446 |
| 152 | 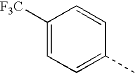 | 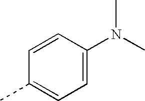 | 3.32 | 19.80 | 495.2226 | 495.2214 |
| 153 | 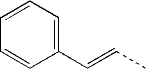 | 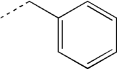 | 7.43 | 23.41 | 424.2242 | 424.2231 |
| 154 | 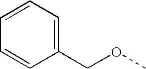 | 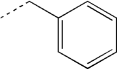 | 6.03* | 23.74 | 428.2209 | 428.2180 |
| 155 | 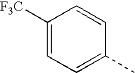 | 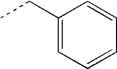 | 3.16 | 17.77 | 466.1968 | 466.1948 |
| 156 | 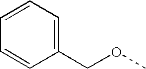 | 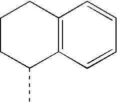 | 9.41* | 25.83 | 468.2483 | 468.2493 |
| 157 | 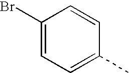 | 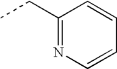 | 3.20 | 18.08 | 477.1145 | 477.1132 |
| 158 | 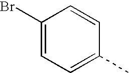 | 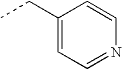 | 3.18 | 17.77 | 477.1143 | 477.1132 |
| 159 | 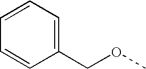 | 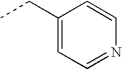 | 3.16 | 17.18 | 429.2146 | 429.2133 |
| 160 | 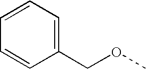 | 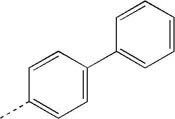 | 19.87* | 28.42 | 490.2324 | 490.2337 |
| 161 | 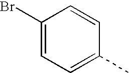 | 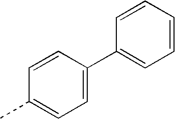 | 24.08* | 29.07 | 538.1342 | 538.1336 |
| 162 | 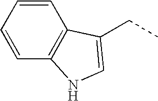 | 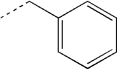 | 4.71 | 21.51 | 451.2330 | 451.2340 |
| 163 | 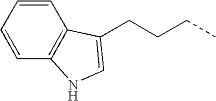 | 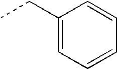 | | | 479.2641 | 479.2653 |
| 164 | 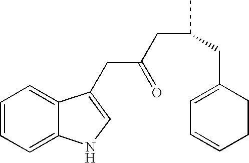 | 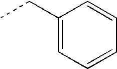 | 9.23 | 24.22 | 598.3010 | 598.3024 |
| 165 | 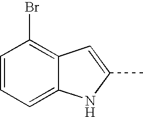 | 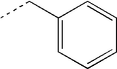 | 15.62 | 25.91 | 515.1272 | 515.1289 |
| 166 | 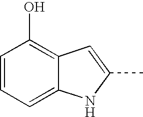 | 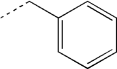 | 4.16 | 20.63 | 453.2126 | 453.2133 |
| 167 | 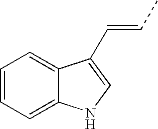 | 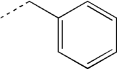 | XX | XX | 463.2360 | 463.2340 |
| 168 | 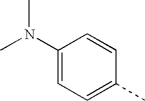 | 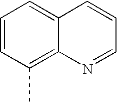 | 3.69 | 19.86 | 479.2420 | 479.2401 |

Selected chemical data for a number of the compounds in tables 1 to 3 is given as follows:

Hydroxamic Acid of example 22 (R⁷=4-Dimethylamino Benzoic Acid): ¹H NMR (d₆-DMSO, 500 MHz): 10.32 (s, 1H); 10.02 (s, 1H); 8.51 (t (5.9 Hz), 1H); 8.14 (d (8.3 Hz), 1H); 7.70 (d (8.7 Hz), 2H); 7.24 to 7.10 (m, 5H); 6.63 (d (8.7 Hz), 2H); 4.52 (m, 1H); 4.21 (d (5.9 Hz), 2H); 2.89 (s, 6H); 2.86 (obsc m (5.2 Hz)); 2.76 (dd (9.5, 13.5 Hz), 1H); 2.44 (m, 2H); 1.94 (t (7.5 Hz), 2H); 1.64 ppm (m, 2H). HRMS calc. for C₂₃H₃₁N₄O₄S (MH⁺): 459.206, Found 459.201.

Hydroxamic Acid of example 24 (R⁷=4-Bromobenzoic Acid): ¹H NMR (d₆-DMSO, 500 MHz): 10.27 (s, 1H); 8.63 (d (7.9 Hz), 1H); 8.56 (t (5.5 Hz), 1H); 7.76 (d (8.7 Hz), 2H); 7.61 (d, 7.9 Hz), 2H); 7.25 to 7.15 (m, 5H); 4.54 (m, 1H); 4.21 (d (5.5 Hz), 2H); 2.90 (dd (4.8, 13.5 Hz), 1H); 2.75 (dd (9.5, 13.5 Hz), 1H); 2.45 (m, 2H); 1.93 (br t (7.1 Hz), 2H); 1.65 ppm (m, 2H). HRMS calc. for C₂₁H₂₅BrN₃O₄S (MH⁺): 494.074, Found 494.076.

Hydroxamic Acid of example 38 (R⁷=2-Napthoic acid): ¹H NMR (d₆-DMSO, 500 MHz): 10.30 (s, 1H); 10.04 (s, 1H); 8.70 (d (7.9 Hz), 1H); 8.60 (t (6.3 Hz), 1H); 8.45 (s, 1H); 7.97 to 7.87 (m, 4H); 7.52 (m, 2H); 7.25 to 7.10 (m, 5H); 4.63 (m, 1H); 4.25 (d (5.5 Hz), 2H); 2.95 (dd (4.8, 13.5 Hz), 1H); 2.83 (ddd (1.6, 9.5, 13.5 Hz), 1H); 2.49 (m, 2H); 1.96 (m, 2H); 1.67 ppm (m, 2H). HRMS calc. for C₂₅H₂₈N₃O₄S (MH⁺): 466.179, Found 466.178.

Hydroxamic Acid of example 40 (R⁷=1H-Indole-2-carboxylic acid): ¹H NMR (d₆-DMSO, 500 MHz): 11.52 (s, 1H); 10.28 (s, 1H); 8.61 (t (6.0 Hz), 1H); 8.53 (d (8.3 Hz), 1H); 7.55 (d (7.9 Hz), 1H); 7.34 (d (7.9 Hz), 1H); 7.25 to 7.20 (m, 6H); 7.10 (t (7.1 Hz), 1H); 6.96 (t (7.1 Hz), 1H); 4.60 (m, 1H); 4.24 (m, 2H); 2.91 (dd (5.5, 13.9 Hz), 1H); 2.77 (dd (9.5, 13.9 Hz), 1H); 2.47 (m, 2H); 1.95 (br t (6.7 Hz), 2H); 1.66 ppm (m, 2H). HRMS calc. for C₂₃H₂₇N₄O₄S (MH⁺): 455.175, Found 455.171.

Hydroxamic Acid of example 48 (R⁷=Cinnamic Acid): ¹H NMR (d₆-DMSO, 500 MHz): 10.29 (s, 1H); 8.62 (t (5.5 Hz), 1H); 8.32 (d (7.9 Hz), 1H); 7.47 (br d (7.13 Hz), 2H); 7.37 to 7.10 (m, 9H); 6.71 (d (15.9 Hz), 1H); 4.52 (dd (7.9, 14.2 Hz), 1H); 4.22 (d (6.3 Hz), 2H); 2.80 (dd (6.3, 13.5 Hz), 1H); 2.65 (dd (7.9, 13.5 Hz), 1H); 2.45 (t (7.1 Hz), 2H); 1.96 (br t (7.9 Hz), 2H); 1.65 ppm (m, 2H). HRMS calc. for C₂₃H₂₈N₃O₄S (MH⁺): 442.179, Found 442.176.

Hydroxamic Acid of example 59 (NR₆XR₇=4-Dimethylamino benzylamine): ¹H NMR (d₆-DMSO, 500 MHz): 10.30 (s, 1H); 8.18 (d (7.9 Hz), 1H); 7.70 (d (8.7 Hz), 2H); 7.42 (br s, 2H); 6.84 (br s, 2H); 6.62 (d (8.7 Hz), 2H); 4.60 (m, 1H); 2.88 (s, 12H); 2.80 (m, 2H); 2.48 (m, 2H); 1.95 (m, 2H); 1.68 ppm (m, 2H). HRMS calc. for C₂₄H₃₄N₅O₄S (MH⁺): 488.2326, Found 488.2301.

Hydroxamic Acid of example 61 (NR₆XR₇=4-Aminobiphenyl): ¹H NMR (d₆-DMSO, 500 MHz): 10.30 (s, 1H); 10.20 (s, 1H); 8.24 (d (7.1 Hz), 1H); 7.72 (d (8.7 Hz), 2H); 7.65 (d (8.7 Hz), 2H); 7.55 (m, 4H); 7.35 (t (7.9 Hz), 2H); 7.24 (t (7.9 Hz), 1H); 6.64 (d (8.7 Hz), 2H); 4.66 (dd (7.9, 14.3 Hz), 1H); 2.92 (obsc m (5.5 Hz)); 2.90 (s, 6H); 2.84 (dd (8.7, 13.5 Hz), 1H); 2.51 (t (7.1 Hz), 2H); 1.98 (m, 2H); 1.69 ppm (m, 2H). HRMS calc. for C₂₈H₃₃N₄O₄S (MH⁺): 521.2217, Found 521.2199.

Hydroxamic Acid of example 65 (NR₆XR₇=8-Aminoquinoline): ¹H NMR (d₆-DMSO, 500 MHz): 10.54 (s, 1H); 10.29 (s, 1H); 8.71 (dd (1.6, 3.9 Hz), 1H); 8.67 (d (7.9 Hz), 1H); 8.56 (d (7.9 Hz), 1H); 8.32 (dd (1.6, 8.3 Hz), 1H); 7.77 (d (8.7 Hz), 2H); 7.60 (d (7.1 Hz), 1H); 7.52 (m, 2H); 6.70 (d (9.1 Hz), 2H); 4.75 (m, 1H); 3.12 (dd (4.8, 13.9 Hz), 1H); 2.92 (s, 6H); 2.88 (m, 1H); 2.47 (m, 2H); 1.97 (t (7.1 Hz), 2H); 1.69 ppm (m, 2H). HRMS calc. for C₂₅H₃₀N₅O₄S (MH⁺): 495.2061, Found 495.2058.

Hydroxamic Acid of example 73 (NR₆XR₇=Benzyl Amine): ¹H NMR (d₆-DMSO, 500 MHz): 10.32 (s, 1H); 10.02 (s, 1H); 8.51 (t (5.9 Hz), 1H); 8.14 (d (8.3 Hz), 1H); 7.70 (d (8.7 Hz), 2H); 7.24 to 7.10 (m, 5H); 6.63 (d (8.7 Hz), 2H); 4.52 (m, 1H); 4.21 (d (5.9 Hz), 2H); 2.89 (s, 6H); 2.86 (obsc m (5.2 Hz)); 2.76 (dd (9.5, 13.5 Hz), 1H); 2.44 (m, 2H); 1.94 (t (7.5 Hz), 2H); 1.64 ppm (m, 2H). HRMS calc. for C₂₃H₃₁N₄O₄S (MH⁺): 459.2061, Found 459.2074.

Hydroxamic Acid of example 96 (NR₆XR₇=^{t}Butyl Amine): ¹H NMR (d₆-DMSO, 500 MHz): 10.28 (s, 1H); 10.00 (s, 1H); 7.90 (d (8.3 Hz), 1H); 7.66 (d (8.7 Hz), 2H); 7.50 (s, 1H); 6.62 (d (8.7 Hz), 2H); 4.44 (m, 1H); 2.89 (s, 6H); 2.77 (dd (5.2, 13.5 Hz), 1H); 2.70 (dd (8.7, 13.1 Hz), 1H); 2.45 (m, 2H); 1.94 (t (6.7 Hz), 2H); 1.64 ppm (m, 2H). HRMS calc. for C₂₀H₃₃N₄O₄S (MH⁺): 425.2217, Found 425.2194.

Selected chemical data for a number of the compounds of examples 122 to 169 is given as follows:

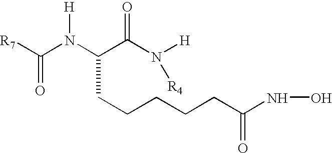

| Compound | | | ¹H NMR |
|---|---|---|---|
| of Example | R₇ | R₄ | 600 MHz |
| 122 | 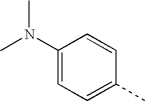 | 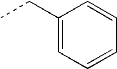 | ¹H NMR (CDCl₃, 500 MHz) δ 8.05 (s, 1 H), 7.69 (d, J = 8.5 Hz, 2 H), 7.36 (d, J = 7.3 Hz, 1 H), 7.23 (m, 5 H), 6.66 (d,, J = 8.5 Hz, 2 H), 4.72 (m, 1 H), 4.47 (dd, J = 14.6, 5.8 Hz, 1 H), 4.29 (dd, J = 14.8, 5.8 Hz, 1 H), 2.99 (s, 6 H), 2.00 (m, 2 H), 1.75 (m, 2 H), 1.45-1.25 (m, 6 H). |
| 126 | 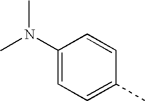 | 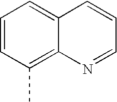 | ¹H NMR (d₆-DMSO, 600 MHz) δ10.54 (s, 1 H), 10.32 (s, 1 H), 8.78 (d, J = 4.3 Hz, 1 H), 8.65 (m, 3 H), 8.42 (dd, J = 8.3, 1.6 Hz, 1 H), 7.86 (d, J =9.0 Hz, 1 H), 7.66 (dd, J = 8.3, 1.2 Hz, 1 H), 7.60 (dd, J = 8.3, 4.2 Hz, 1 H), 7.58 (t, J = 8.0 Hz, 1 H), 6.7 (d, J =9.0 Hz, 2 H), 4.61 (m, 1 H), 1.93 (t, J = 7.5 Hz, 2 H), 1.49 (m, 2 H), 1.48-1.45 (m, 2 H), |
| # 1.31-1.23 (m, 4 H). | | | |
| 130 | 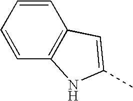 | 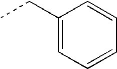 | ¹H NMR (d₆-Acetone, 500 MHz) δ10.76 (s, 2 H), 9.93 (s, 1 H), 7.87 (t, J =6.0 Hz, 1 H), 7.81 (d, J = 8.0 Hz, 1 H), 7.61 (d, J = 8.0 Hz, 1 H), 7.3-7.1 (m, 5 H), 7.06 (t, J = 8 Hz, 1 H), 4.64 (m, 1 H), 4.42 (d, J = 6 Hz, 2 H), 4.39 (s, 1 H), 1.96 (m, 2 H), 1.80 (m, 2 H), 1.60 (m, 2 H), 1.4-1.3 (m, 4 H). |
| 132 | 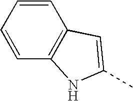 | 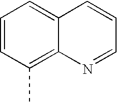 | ¹H NMR (d₆-DMSO, 600 MHz) δ11.65 (s, 1 H), 10.52 (s, 1 H), 10.32 (s, 1 H), 8.99 (d, J = 7.4 Hz, 1 H), 8.78 (m, 1 H), 8.65 (dd, J = 7.9, 1.1 Hz, 1 H), 8.64 (s, 1 H), 8.39 (dd, J = 8.3, 1.6 Hz, 1 H), 7.69-7.66 (m, 2 H), 7.60-7.57 (m, 2 H), 7.43 (d, J = 8.3 Hz, 1 H), 7.39 (s, 1 H), 7.20 (t, J = 7.9 Hz, 1 H), 7.06 (t, J = 7.8 Hz, 1 H), 4.72 (m, 1 H), 1.93 (t, J = 7.3 |
| # Hz, 2 H), 1.50 (m, 4 H), 1.48-1.30 (m, 4 H). | | | |
| 136 | 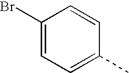 | 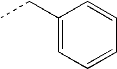 | ¹H NMR (d₆-Acetone, 500 MHz) δ9.92 (s, 2 H), 7.88 (d, J = 8.5 Hz, 2 H), 7.84 (d, J = 6.5, Hz, 2 H), 7.65 (d, J =8.5 Hz, 2 H), 7.28 (m, 3 H), 7.21 (m, 1 H), 4.62 (m, 1 H), 4.42 (d, J = 6 Hz, 2 H), 2.72 (s, 1 H), 1.94 (m, 2 H), 1.79 (m, 2 H), 1.58 (m, 2 H), 1.4-1.3 (m, 4 H). |
| 139 | 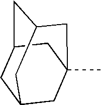 | 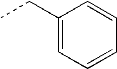 | ¹H NMR (d₆-Acetone, 500 MHz) δ9.95 (s, 1 H), 7.93 (s, 1 H), 7.67 (br s, 1 H), 7.28-7.20 (m, 5 H), 6.69 (d, J =8.0 Hz, 1 H), 4.44 (m, 1 H), 4.39 (d, J =6.0 Hz, 2 H), 1.99 (br s, 3 H), 1.87 (m, 6 H), 1.82 (m, 2 H), 1.72 (m, 6 H), 1.65-1.51 (m, 4 H), 1.32 (m, 4 H). |
| 140 | 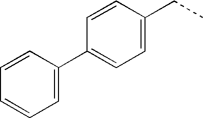 | 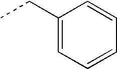 | ¹H NMR (d₆-Acetone, 500 MHz) δ9.91 (s, 1 H), 7.80 (s, 1 H), 7.68 (m, 2 H), 7.64 (d, J = 7.5 Hz, 2 H), 7.57 (d, J = 8.2 Hz, 2 H), 7.46-7.38 (m, 5 H), 7.35-7.20 (5 H), 4.44 (m, 1 H), 4.37 (d, J = 6.0 Hz, 2 H), 3.62 (d, J =4.2 Hz, 2 H), 1.81 (m, 2 H), 1.62 (m, 2 H), 1.53 (m, 2 H), 1.30 (m, 4 H). |
| 141 | 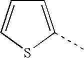 | 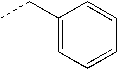 | ¹H NMR (d₆-Acetone, 500 MHz) δ9.90 (s, 1 H), 7.85 (br s, 1 H), 7.81 (dd, J = 3.7, 1.1 Hz, 1 H), 7.74 (br s, 1 H), 7.67 (dd, J = 5.0, 1.1 Hz, 1 H), 7.29 (m, 5 H), 7.21 (br s, 1 H), 7.12 (dd, J = 5.0 Hz, 1 H), 4.58 (m, 1 H), 4.41 (d, J = 6.0 Hz, 2 H), 1.76 (m, 2 H), 1.57 (m, 2 H), 1.46-1.30 (m, 6 H). |
| 142 | 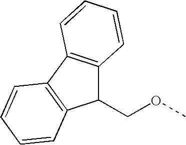 | 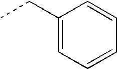 | ¹H NMR (d₆-Acetone, 500 MHz) δ9.91 (s, 1 H), 7.85 (d, J = 7.8 Hz, 2 H), 7.77 (br s, 1 H), 7.70 (m, 4 H), 7.40 (t, J = 7.5 Hz, 2 H), 7.29 (m, 5 H), 7.21 (br s, 1 H), 6.61 (d, J = 7.6 Hz, 1 H), 4.41 (d, J = 5.9 Hz, 2 H), 4.33 (m, 1 H), 4.22 (d, J = 6.9 Hz, 2 H), 4.17 (m, 1 H), 1.84 (m, 2 H), 1.68 (m, 2 H), 1.59 (m, 2 H), 1.46-1.31 (m, 6 H). |
| 146 | 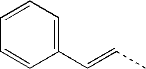 | 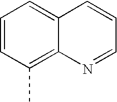 | ¹H NMR (d₆-DMSO, 600 MHz) δ10.46 (s, 1 H), 10.31 (s, 1 H), 8.86 (dd, J = 4.3, 1.7 Hz, 1 H), 8.64 (dd, J =7.7, 1.3 Hz, 1 H), 8.41 (dd, J = 8.3, 1.7 Hz, 1 H), 8.31 (dd, J = 8.5, 3.6 Hz, 1 H), 7.69 (dd, J = 8.3, 1.3 Hz, 1 H), 7.59-7.55 (m, 2 H), 7.50 (d, J =15.8 Hz, 1 H), 7.65 (dd, J = 8.3, 4.2 Hz, 1 H), 7.59 (t, J = 8.0 Hz, 1 H), 7.44-7.7.39 (m, 3 H), 6.84 (d, |
| # J =15.8 Hz, 1 H), 4.11 (m, 1 H) 1.93 (t, J =7.5 Hz, 2 H), 1.64-1.58 (m, 2 H), 1.46 (m, 2 H), 1.34-1.27 (m, 4 H). | | | |
| 147 | 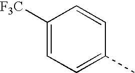 | 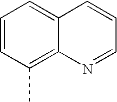 | ¹H NMR (d₆-DMSO, 600 MHz) δ10.56 (s, 1 H), 10.31 (s, 1 H), 9.28 (d, J = 7.4 Hz, 2 H), 8.84 (dd, J = 4.1, 1.4 Hz, 1 H), 8.63 (dd, J = 7.7, 1.3 Hz, 1 H), 8.42 (dd, J = 8.3, 1.6 Hz, 1 H), 8.12 (d, J = 7.1 Hz, 1 H), 7.83 (d, J 7.3 Hz, 2 H), 7.69 (dd, J = 8.3, 1.2 Hz, 1 H), 7.63 (dd, J = 8.2, 4.2 Hz, 1 H), 7.59 (t, J = 7.9 Hz, 1 H), 4.10 (m, 1 H) 1.93 (t, |
| # J = 7.5 Hz, 2 H), 1.64-1.58 (m, 2 H), 1.46 (m, 2 H), 1.34-1.27 (m, 4 H). | | | |
| 148 | 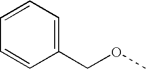 | 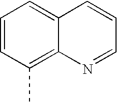 | ¹H NMR (d₆-DMSO, 600 MHz) δ10.46 (s, 1 H), 10.31 (s, 1 H), 8.86 (dd, J = 4.1, 1.4 Hz, 1 H), 8.64 (d, J =7.1 Hz, 1 H), 8.42 (dd, J = 8.3, 1.6 Hz, 1 H), 8.12 (d, J = 7.1 Hz, 1 H), 7.69 (dd, J = 8.3, 1.2 Hz, 1 H), 7.66 (dd, J = 8.2, 4.2 Hz, 1 H), 7.59 (t, J =7.9 Hz, 1 H), 7.37 (m, 3 H), 7.30 (m, 2 H), 5.01 (m, 2 H), 4.10 (m, 1 H), 1.93 (t, J = 7.5 Hz, 2 H), |
| # 1.64-1.58 (m, 2 H), 1.46 (m, 2 H), 1.34-1.27 (m, 4 H). | | | |
| 149 | 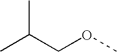 | 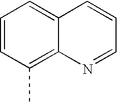 | ¹H NMR (d₆-DMSO, 600 MHz) δ10.46 (s, 1 H), 10.31 (s, 1 H), 8.86 (d, J = 3.8 Hz, 1 H), 8.64 (d, J = 7.1 Hz, 1 H), 8.42 (dd, J = 8.3, 1.6 Hz, 1 H), 7.94 (d, J = 7.1 Hz, 1 H), 7.68 (dd, J =8.3, 1.2 Hz, 1 H), 7.65 (dd, J = 8.3, 4.2 Hz, 1 H), 7.59 (t, J = 8.0 Hz, 1 H), 4.16 (m, 1 H), 3.88 (m, 2 H), 2.41 (m, 1 H), 1.92 (t, J = 7.5 Hz, 2 H), 1.83 (m, 2 H), 1.46 |
| # (m, 2 H), 1.35-1.28 (m, 4 H), 0.91 (dd, J = 6.4, 3.8 Hz, 6 H) | | | |
| 150 | 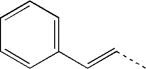 | 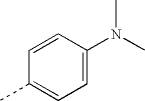 | ¹H NMR (d₆-DMSO, 600 MHz) δ10.33 (s, 1 H), 10.08 (s, 1 H), 8.36 (d, J = 7.8 Hz, 2 H), 7.56 (m, 3 H), 7.35 (m, 5 H), 7.06 (br s, 1 H), 6.80 (d, J =15.8 Hz, 1 H), 4.51 (m, 1 H), 4.04 (br s, 1 H), 1.93 (t, J = 7.5 Hz, 2 H), 1.72-1.58 (m, 2 H), 1.46 (m, 2 H), 1.36-1.27 (m, 4 H). |
| 151 | 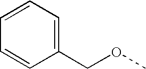 | 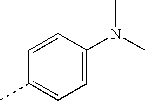 | ¹H NMR (d₆-DMSO, 600 MHz) δ10.32 (s, 1 H), 9.94 (s, 1 H), 7.55 (d, J = 7.8 Hz, 2 H), 7.35 (m, 5 H), 7.30 (d, J = 7.8 Hz, 2 H), 7.06 (br s, 1 H), 5.02 (s, 2 H), 4.10 (m, 1 H), 4.04 (br s, 1 H), 1.93 (t, J = 7.5 Hz, 2 H), 1.64-1.58 (m, 2 H), 1.46 (m, 2 H), 1.34-1.27 (m, 4 H. |
| 152 | 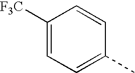 | 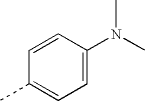 | ¹H NMR (d₆-DMSO, 600 MHz) δ10.32 (s, 1 H), 10.06 (s, 1 H), 8.8 (d, J =7.6 Hz, 2 H), 8.15 (d, J = 8.2 Hz, 2 H), 7.85 (d, J = 8.2 Hz, 2 H), 7.55 (d, J = 8.3 Hz, 2 H), 7.05 (br s, 1 H), 4.55 (m, 1 H), 4.04 (br s, 1 H), 1.93 (t, J = 7.5 Hz, 2 H), 1.49 (m, 2 H), 1.42 (m, 1 H), 1.34-1.27 (m, 3 H). |
| 153 | 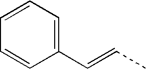 | 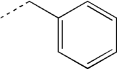 | ¹H NMR (d₆-DMSO, 600 MHz) δ10.32 (s, 1 H), 8.64 (s, 1 H), 8.54 (t, J =6.0 Hz 1 H), 8.24 (d, J = 8.4 Hz, 1 H), 7.55 (d, J = 7.2 Hz, 2 H), 7.44-7.36 (m, 3 H), 7.31-7.22 (m, 5 H), 6.78 (d, J = 15.8 Hz, 1 H), 4.40 (m, 1 H), 4.28 (d, J = 5.5 Hz, 2 H), 1.91 (t, J =7.5 Hz, 2 H), 1.69 (m, 1 H), 1.56 (m, 1 H), 1.45 (m, 2 H), 1.31-1.23 (m, 4 H). |
| 154 | 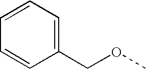 | 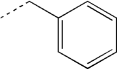 | ¹H NMR (d₆-DMSO, 600 MHz) δ10.31 (s, 1 H), 8.40 (s, 1 H), 7.41 (d, J =8.1 Hz, 2 H), 7.36-7.29 (m, 6 H), 7.23 (d, J = 7.5 Hz, 2 H), 5.02 (d, J =3.5 Hz, 2 H), 4.27 (dd, J = 5.6, 3.7 Hz, 2 H), 3.97 (m 1 H), 3.44 (br s, 1 H), 1.91 (t, J = 7.4 Hz, 2 H), 1.69 (m, 1 H), 1.56 (m, 1 H), 1.45 (m, 2 H), 1.31-1.23 (m, 4 H). |
| 155 | 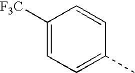 | 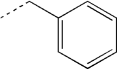 | ¹H NMR (d₆-DMSO, 600 MHz) δ10.31 (s, 1 H), 8.52 (t, J = 7.2 Hz, 1 H), 8.1 (d, J = 8.1 Hz, 2 H), 7.85 (d, J = 8.2 Hz, 2 H), 7.64 (br s, 1 H), 7.32-7.22 (m, 5 H), 4.40 (m, 1 H), 4.28 (d, J = 7.2 Hz, 2 H), 1.91 (t, J = 7.5 Hz, 2 H), 1.77 (m, 2 H), 1.47 (m, 2 H), 1.37-1.26 (m, 4 H). |
| 156 | 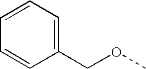 | 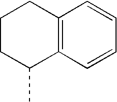 | ¹H NMR (d₆-DMSO, 600 MHz) δ10.31 (s, 1 H), 8.25 (d, J = 8.4 Hz, 1 H), 8.16 (d, J = 7.2 Hz, 1 H), 7.35 (m, 2 H), 7.29 (m, 2 H), 7.13 (m, 3 H), 5.03 (m, 2 H), 4.94 (m, 1 H), 3.97 (m, 1 H), 2.71 (m, 2 H), 1.91 (t, J = 7.5 Hz, 2 H), 1.84 (m, 2 H), 1.69-1.54 (m, 4 H), 1.45 (m, 2 H), 1.31-1.23 (m, 4 H). |
| 157 | 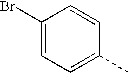 | 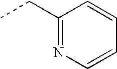 | ¹H NMR (d₆-DMSO, 600 MHz) δ10.32 (s, 1 H), 8.68 (t, J = 5.6 Hz, 1 H), 8.64 (d, J = 7.6 Hz, 1H), 8.60 (d, J =4.8 Hz, 1 H), 8.02 (t, J = 7.4 Hz, 1 H), 7.85 (d, J = 6.7 Hz, 2 H), 7.69 (d, J = 11.0 Hz, 2 H), 7.47 (m, 2 H), 4.44 (d, J = 5.9 Hz, 2 H), 4.42 (m, 1 H), 1.92 (t, J = 7.5 Hz, 2 H), 1.77-1.73 (m, 2 H), 1.47 (m, 2 H), 1.36-1.26 (m, 4 H) |
| 158 | 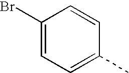 | 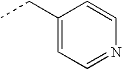 | ¹H NMR (d₆-DMSO, 600 MHz) δ10.32 (s, 1 H), 8.73 (m, 3 H), 8.66 (d, J = 7.3 Hz, 2 H), 7.85 (d, J = 6.7 Hz, 2 H), 7.69 (d, J = 11.0 Hz, 2 H), 7.65 (d, J = 4.9 Hz, 2 H), 4.46 (m, 2 H), 4.40 (m, 1 H), 1.92 (t, J = 7.5 Hz, 2 H), 1.77-1.73 (m, 2 H), 1.47 (m, 2 H), 1.36-1.26 (m, 4 H) |
| 159 | 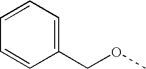 | 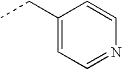 | ¹H NMR (d₆-DMSO, 600 MHz) δ10.32 (s, 1 H), 8.69 (m, 3 H), 7.61 (m, 2 H), 7.54 (d, J = 7.5 Hz, 1 H), 7.36-7.31 (m, 5 H), 5.05 (m, 2 H), 4.45 (m, 2 H), 3.99 (m, 1 H), 1.92 (t, J = 7.5 Hz, 2 H), 1.64 (m, 1 H), 1.56 (m, 1 H), 1.46 (m, 2 H), 1.36-1.26 (m, 4 H) |
| 160 | 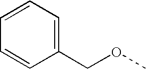 | 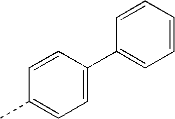 | ¹H NMR (d₆-DMSO, 600 MHz) δ10.32 (s, 1 H), 10.12 (s, 1 H), 8.64 (m, 1 H), 7.69 (m 2 H), 7.63 (m, 3 H), 7.58 (d, J = 7.8 Hz, 2 H), 7.44 (t, J = 7.8 Hz, 2 H), 7.36 (m, 3 H), 7.32 (m, 2 H), 5.03 (s, 2 H), 4.14 (m, 1 H), 1.92 (t, J =7.5 Hz, 2 H), 1.66 (m, 1 H), 1.61 (m, 1 H), 1.46 (m, 2 H), 1.37-1.26 (m, 4 H) |
| 161 | 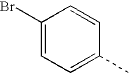 | 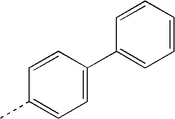 | ¹H NMR (d₆-DMSO, 600 MHz) δ10.32 (s, 1 H), 10.22 (s, 1 H), 8.71 (d, J = 7.5 Hz, 1 H), 7.87 (d, J = 8.6 Hz, 2 H), 7.70 (t, J = 9.0 Hz, 4 H), 7.63 (m, 4 H), 7.44 (t, J = 7.8 Hz, 2 H), 7.32 (t, J = 7.9 Hz, 1 H) 4.55 (m, 1 H), 1.93 (t, J = 7.5 Hz, 2 H), 1.80 (m, 2 H), 1.49 (m, 2 H), 1.43 (m, 1 H), 1.37 (m, 1 H), 1.26 (m, 2 H) |
| 162 | 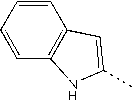 | 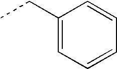 | ¹H NMR (d₆-DMSO, 600 MHz) δ10.82 (s, 1 H), 10.32 (s, 1 H), 8.42 (t, J = 6.0 Hz, 1 H), 8.02 (d, J = 8.1 Hz, 1 H), 7.54 (d, J = 7.6 Hz, 1 H), 7.32 (d, J = 8.1 Hz, 1 H), 7.29-7.18 (m, 5 H), 7.05 (dd,, J = 7.0, 1.1 Hz, 1 H), 6.94 (dd,, J = 7.0, 1.1 Hz, 1 H), 4.24 (m, 1 H), 3.56 .92 (t, J = 7.5 Hz, 2 H), 1.66 (m, 1 H), 1.61 (m, 1 H), 1.44 (m, 2 H), 1.31-1.18 (m, 4 H) |

Biological Data

The cytotoxicities of the compounds of the invention were determined by clonogenic survival of human cancer cells (MM96L, melanoma) and human normal cells (NFF, neonatal foreskin fibroblasts). Cells were incubated with the compounds at various concentrations of compound (0.01-10 μg/mL) for 24 hours, washed, and then grown for a further four days in the absence of hydroxamic acid before determining cell survival by cell count. The final readout involved staining with sulforhodamine B (SRB), a cost-effective method amenable to automation and high throughput analysis. At the technical level, “cell sensitivity” is often inferred from short term (1-2 day) observations such as apoptosis, which may not be a satisfactory model of clonogenic survival. Compounds were considered for further testing if they exhibited either potency (IC₅₀ 200 nM) or selectivity (SI>5) in their killing of cancer cells over normal cells.

Cell Lines and Culture Medium. All cell lines used in this study have been described previously (Parsons et al., 1986; Todaro et al., 1980; Glenn et al., 2004). All cell lines were cultured in 10% heat-inactivated foetal calf serum (CSL, Australia) in RPMI 1640 medium supplemented with 100 U/mL penicillin, 100 μg/mL streptomycin, and 3 mM HEPES at 5% CO₂, 99% humidity at 37° C. Primary human fibroblasts were obtained from neonatal foreskins and cultured in the above medium. Routine mycoplasma tests were performed using Hoechst stainⁱ and were always negative.

Cell Survival Assay. Cells were plated into 96-well microtitre plates at 5×10³ cells/well, and allowed to adhere overnight. Test compounds were added to culture medium at the indicated concentrations, and plates incubated in the above conditions for 24 hours. Following this incubation period, compounds and media were removed, and replaced with fresh culture medium. Cells were then grown for a further 72 hours before assay using sulforhodamine B (SRB; Sigma, St. Louis, Mo.) as previously described. Briefly, the culture medium was removed from the 96-well microtitre plates and the plates washed twice with phosphate buffered saline (PBS), before the cells were fixed with methylated spirits for 15 minutes. The plates were then rinsed with tap water and the fixed cells stained with 50 μL/well of SRB solution (0.4% sulforhodamine B (w/v) in 1% (v/v) acetic acid) over a period of 1 hour. The SRB solution was then removed from the wells and the plates rapidly washed two times with 1% (v/v) acetic acid. Protein bound dye was then solubilised with the addition of 100 μL of 10 mM unbuffered Tris, and incubated for 15 min at 25° C. Plates were then read at 564 nm on a VERSA max tunable microplate reader (Molecular Devices, Sunnyvale, Calif.).

The results of the biological test results on each of the compounds is as given in the following tables.

TABLE 5
Activity of Compounds of Examples 22-58

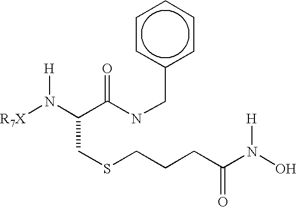

| Compound | | | | IC₅₀ MM96L | |
|---|---|---|---|---|---|
| of Example | R₇—X | Log D_{7.0} | IC₅₀ NFF (μM) | (μM) | Selectivity |
| 22 | 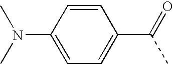 | 2.1 | 0.35±0.07 | 0.14±0.09 | 2.5 |
| 23 | 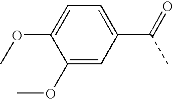 | 1.6 | 8.3±0.8 | 1.7±0.1 | 4.9 |
| 24 | 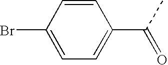 | 2.7 | 0.83±0.09 | 0.02±0.1 | 4.2 |
| 25 | 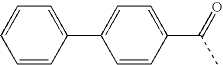 | 3.5 | 2.8±0.2 | 0.9±0.1 | 3.1 |
| 26 | 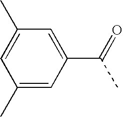 | 2.8 | 10.9±0.9 | 2.0±0.4 | 5.5 |
| 27 | 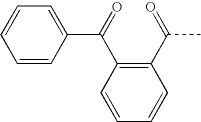 | 3.0 | 30±1 | 24±3 | 1.3 |
| 28 | 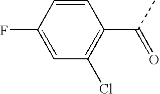 | 2.2 | >100 | 28±3 | >3 |
| 29 | 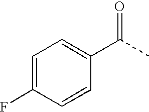 | 1.5 | 26±1 | 5.2±0.6 | 5.0 |
| 30 | 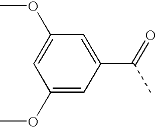 | 1.5 | 4.5±0.6 | 1.7±0.3 | 2.6 |
| 31 | 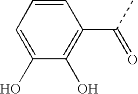 | 0.7 | 4.5±0.6 | 32±3 | 0.14 |
| 32 | 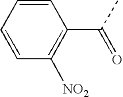 | 0.9 | >100 | 10.6±0.1 | >10 |
| 33 | 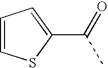 | 1.8 | 9±1 | 2.5±0.2 | 3.6 |
| 34 | 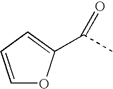 | 0.5 | 22±1 | 7.2±0.2 | 3.1 |
| 35 | 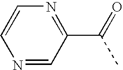 | 0.4 | 62±5 | 19±2 | 3.3 |
| 36 | 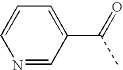 | 0.1 | >100 | 12.8±0.8 | >8 |
| 37 | 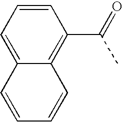 | 2.8 | 5.3±0.6 | 6.3±0.6 | 0.8 |
| 38 | 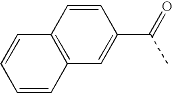 | 2.8 | 1.14±0.05 | 0.6±0.2 | 1.9 |
| 39 | 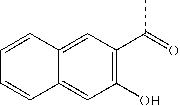 | 2.5 | >100 | 13±2 | >8 |
| 40 | 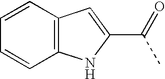 | 1.4 | 0.8±0.2 | 0.13±0.09 | 6.2 |
| 41 | 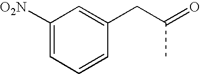 | 1.0 | >100 | 12±1 | >8 |
| 42 | 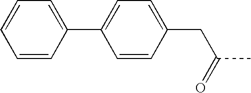 | 3.5 | 21±2 | 12±3 | 1.8 |
| 43 | 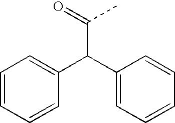 | 2.6 | 22±2 | 9.3±0.3 | 2.4 |
| 44 | 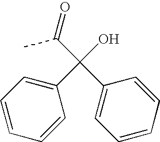 | 2.9 | 9.3±0.7 | 1.8±0.2 | 5.2 |
| 45 | 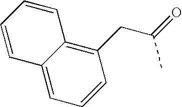 | 2.8 | 15±1 | 4.1±0.7 | 3.7 |
| 46 | 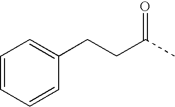 | 1.5 | 21±3 | 7.4±0.5 | 2.9 |
| 47 | 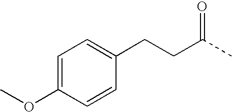 | 1.4 | 16±1 | 8±1 | 2.0 |
| 48 | 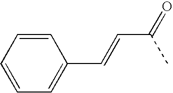 | 2.2 | 0.8±0.2 | 0.2±0.1 | 4.0 |
| 49 | 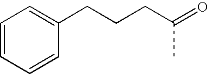 | 2.6 | 11±2 | 5±1 | 2.2 |
| 50 | 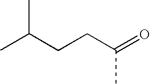 | 1.8 | 25±4 | 7±2 | 3.6 |
| 51 | 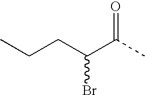 | 2.4 | 10±1 | 5±2 | 2.0 |
| 52 | 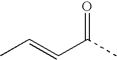 | 0.4 | >100 | 11±0.7 | >9 |
| 53 | 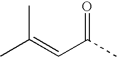 | 0.8 | 22±1 | 11±1 | 2.0 |
| 54 | 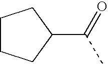 | 1.0 | >100 | 21±3 | >5 |
| 55 | 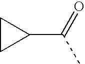 | 0 | >100 | 21±2 | >5 |
| 56 | 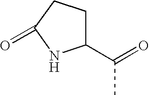 | 0.9 | 9.6±0.9 | 4.4±0.3 | 2.2 |
| 57 | 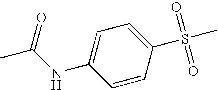 | 0.6 | >100 | 45±7 | >2 |
| 58 | 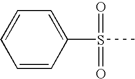 | 1.7 | >100 | <100 | >1 |

TABLE 6
Activity of Compounds of Examples 59-96

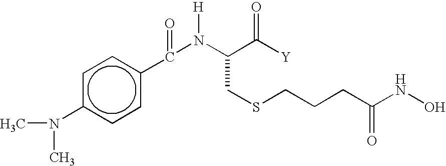

| Compound | | | IC₅₀ (μM) | | |
|---|---|---|---|---|---|
| of Example | Y | Log D_{7.0} | NFF | MM96L | Selectivity |
| 59 | 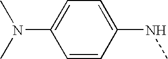 | 1.6 | 0.6±0.1 | 0.1±0.1 | 6.0 |
| 60 | 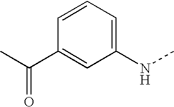 | 1.3 | 1.60±0.08 | 0.55±0.05 | 2.9 |
| 61 | 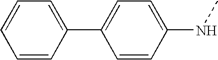 | 3.6 | 0.32±0.05 | 0.17±0.05 | 1.9 |
| 62 | 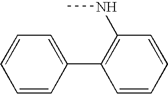 | 3.6 | 4.4±0.6 | 2.1±0.2 | 2.1 |
| 63 | 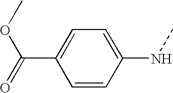 | 2.2 | 2.8±0.1 | 0.96±0.07 | |
| 64 | 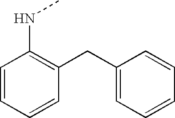 | 4.5 | 6.8±0.4 | 3.5±0.2 | 1.9 |
| 65 | 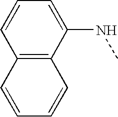 | 2.9 | 5.9±0.7 | 1.3±0.1 | 4.5 |
| 66 | 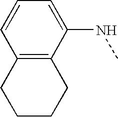 | 3.2 | 2.2±0.3 | 0.5±0.1 | 4.4 |
| 67 | 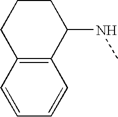 | 2.4 | 2.2±0.2 | 0.20±0.1 | 11 |
| 68 | 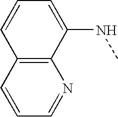 | 2.5 | 3.0±0.3 | 0.61±0.08 | 5.0 |
| 69 | 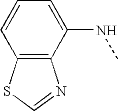 | 0.2 | 3.3±0.3 | 1.3±0.1 | 2.5 |
| 70 | 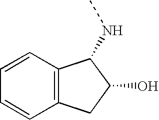 | 0.6 | 8.2±0.8 | 0.90±0.05 | 9.1 |
| 71 | 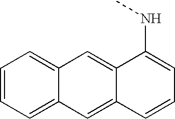 | 4.1 | 2.2±0.3 | 1.7±0.3 | 1.3 |
| 72 | 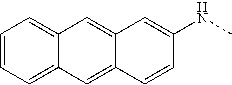 | 4.1 | 1.14±0.06 | 0.55±0.07 | 2.1 |
| 73 | 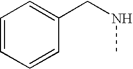 | 1.3 | 0.35±0.07 | 0.14±0.09 | 2.5 |
| 74 | 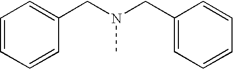 | 3.1 | 15.3±0.6 | 2.3±0.2 | 6.5 |
| 75 | 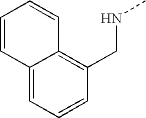 | 2.5 | 0.42±0.05 | 0.20±0.02 | 2.1 |
| 76 | 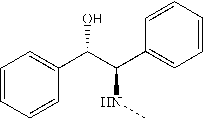 | 2.2 | 9±3 | 2.1±0.2 | 4.3 |
| 77 | 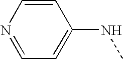 | 0.4 | 7.2±0.8 | 1.8±0.2 | 4.1 |
| 78 | 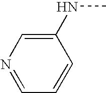 | 0.4 | 11±1 | 2.1±0.1 | 5.6 |
| 79 | 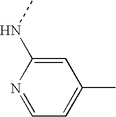 | 1.2 | 11.2±0.7 | 1.12±0.06 | 10.0 |
| 80 | 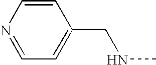 | 0.1 | 14±1 | 2.2±0.3 | 6.2 |
| 81 | 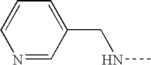 | 0.1 | 8.8±0.5 | 1.49±0.09 | 5.9 |
| 82 | 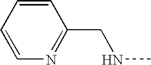 | 0.2 | 6.5±0.3 | 1.2±0.1 | 5.5 |
| 83 | 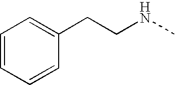 | 1.7 | 2.7±0.3 | 1.4±0.1 | 1.9 |
| 84 | 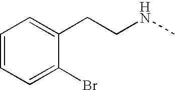 | 2.6 | 3.3±0.3 | 0.7±0.2 | 4.7 |
| 85 | 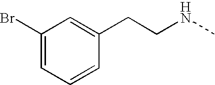 | 2.7 | 3.1±0.1 | 0.9±0.1 | 3.4 |
| 86 | 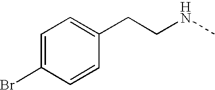 | 2.6 | 2.6±0.3 | 1.0±0.1 | 2.6 |
| 87 | 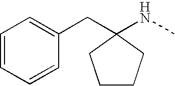 | 3.1 | 4.2±0.5 | 0.6±0.1 | 7.0 |
| 88 | 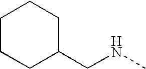 | 2.1 | 3.6±0.2 | 0.51±0.09 | 7.1 |
| 89 | 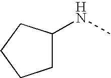 | 1.9 | 2.6±0.3 | 1.5±0.1 | 1.7 |
| 90 | 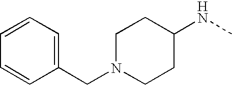 | 0.4 | 1.28±0.08 | 1.5±0.2 | 0.9 |
| 91 | 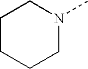 | 0.9 | 20±2 | 4.9±0.3 | 4.2 |
| 92 | 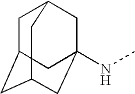 | 2.3 | 7.1±0.5 | 1.48±0.08 | 4.8 |
| 93 | 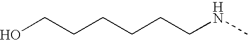 | 0.8 | 12±1 | 1.8±0.2 | 6.7 |
| 94 | 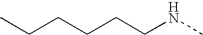 | 1.7 | 7±1 | 0.85±0.04 | 8.0 |
| 95 | 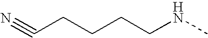 | 1.4 | 6.0±0.8 | 1.3±0.2 | 4.5 |
| 96 | 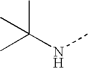 | 0.9 | 3.5±0.5 | 0.7±0.2 | 5.0 |

TABLE 7
Activity of Compounds of Examples 97-102

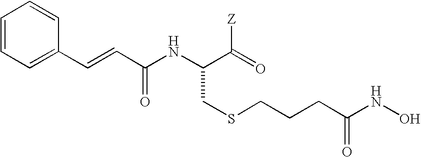

| Com- | | | | | |
|---|---|---|---|---|---|
| pound | | | | | |
| of Ex- | | Log | IC50 (μM) | | Selec- |
| ample | Z | D_{7.0} | NFF | MM96L | tivity |
| 97 | 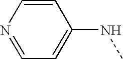 | 0.7 | 1.6 ± 0.3 | 0.5 ± 0.2 | 3.3 |
| 98 | 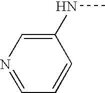 | 0.7 | 6.4 ± 0.8 | 1.2 ± 0.3 | 5.2 |
| 99 | 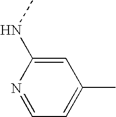 | 1.5 | 4.0 ± 0.6 | 0.6 ± 0.2 | 6.5 |
| 100 | 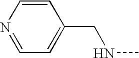 | 0.4 | 1.6 ± 0.2 | 0.34 ±0.02 | 4.8 |
| 101 | 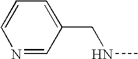 | 0.4 | 13.2 ± 0.7 | 0.9 ± 0.2 | 15.1 |
| 102 | 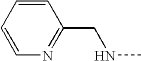 | 0.5 | 4.2 ± 0.4 | 0.8 ± 0.3 | 5.3 |

TABLE 8
Activity of Compounds of Example 103-121

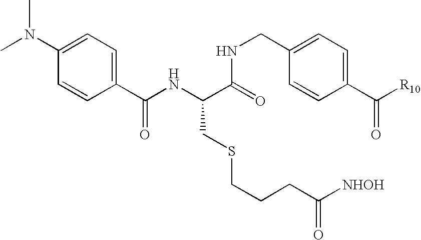

| Compound of | | | IC50 (μM) | | |
|---|---|---|---|---|---|
| Example | R₁₀ | Log D_{7.0} | NFF | MM96 | Selectivity |
| 103 | 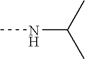 | 1.44 | 10.12 | 1.38 | 7.30 |
| 104 | 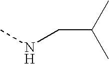 | 1.71 | 8.08 | 1.62 | 5.0 |
| 105 | 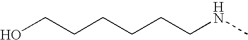 | 1.02 | 17.03 | 4.26 | 4.0 |
| 106 | 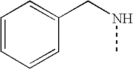 | 2.05 | 11.84 | 1.18 | 10.0 |
| 107 | 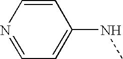 | 0.83 | 16.89 | 2.96 | 5.7 |
| 108 | 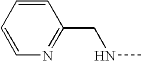 | 0.95 | 16.89 | 2.28 | 7.4 |
| 109 | 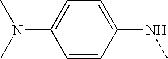 | 2.23 | 2.42 | 0.54 | 4.5 |
| 110 | 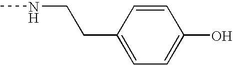 | 1.96 | 16.10 | 2.98 | 5.4 |
| 111 | 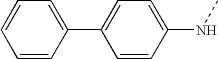 | 4.21 | 1.11 | 0.51 | 2.2 |
| 112 | 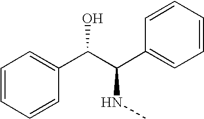 | 3.00 | 14.34 | 2.87 | 5.0 |
| 113 | 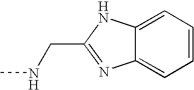 | 1.52 | 15.84 | 3.96 | 4.0 |
| 114 | 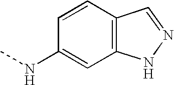 | 1.73 | 12.15 | 1.5 | 8.1 |
| 115 | 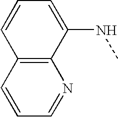 | 3.80 | 1.35 | 0.56 | 2.4 |
| 116 | 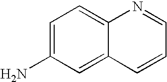 | 2.41 | 5.57 | 2.23 | 2.5 |
| 117 | 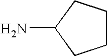 | 2.00 | 5.27 | 1.19 | 4.4 |
| 118 | 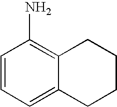 | — | 3.78 | 1.17 | 3.2 |
| 119 | 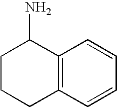 | — | 1.74 | 0.93 | 1.9 |
| 120 | 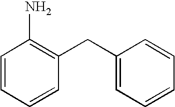 | — | 2.32 | 1.02 | 2.4 |
| 121 | 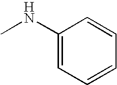 | — | 11.57 | 1.41 | 8.2 |

TABLE 9
Activity of Compounds of Examples 122-171

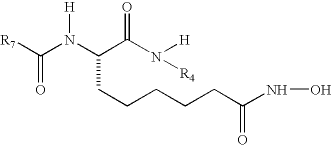

| Compound | | | | | IC50 (uM) | | |
|---|---|---|---|---|---|---|---|
| Of Example | R₇ | R₄ | * | LogD | NFF | MM96L | SI^{a} |
| 122 | 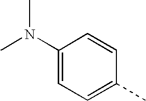 | 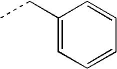 | S | 2.8 | 12 ± 2 | 1.6 ± 0.3 | 7.8 |
| 123 | 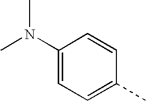 | 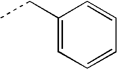 | R | 2.8 | 1.0 ± 0.1 | 0.16 ± 0.08 | 6.0 |
| 124 | 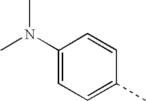 | 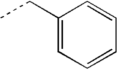 | rac | 2.8 | 0.87 ± 0.07 | 0.13 ± 0.02 | 6.7 |
| 125 | 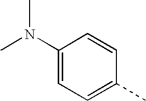 | 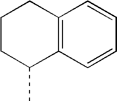 | rac | 3.6 | 1.81 ± 0.07 | 0.3 ± 0.1 | 6.0 |
| 126 | 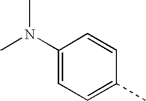 | 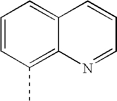 | S | 2.8 | 0.57 ± 0.07 | 0.02 ± 0.01 | 28 |
| 127 | 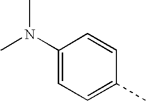 | 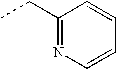 | S | 1.9 | 33 ± 5 | 3.6 ± 0.4 | 9.1 |
| 128 | 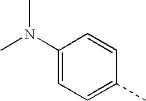 | 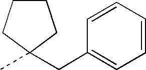 | S | 4.1 | 3.0 ± 0.3 | 0.9 ± 0.2 | 3.5 |
| 129 | 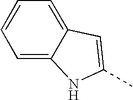 | 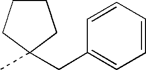 | S | 3.4 | 1.6 ± 0.2 | 0.9 ± 0.2 | 1.7 |
| 130 | 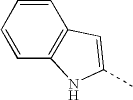 | 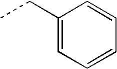 | S | 2.45 | 1.260 | 0.252 | 5.0 |
| 131 | 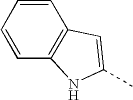 | 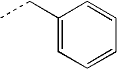 | R | 2.45 | 0.572 | 0.080 | 7.1 |
| 132 | 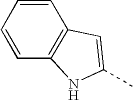 | 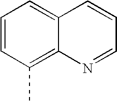 | S | 2.96 | 0.337 | 0.021 | 16 |
| 133 | 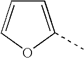 | 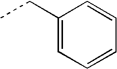 | rac | 1.1 | 4.6 ± 0.4 | 0.2 ± 0.1 | 23 |
| 134 | 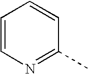 | 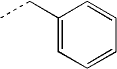 | rac | 1.2 | 24 ± 8 | 9.5 ± 0.8 | 2.5 |
| 135 | 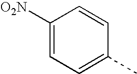 | 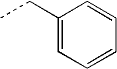 | rac | 0.9 | 9 ± 2 | 6 ± 1 | 1.5 |
| 136 | 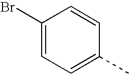 | 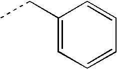 | S | 3.02 | 5.864 | 1.994 | 4.2 |
| 137 | 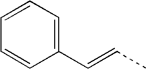 | 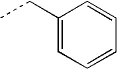 | S | 2.67 | 4.014 | 0.46 | 8.7 |
| 138 | 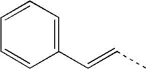 | 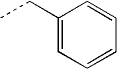 | R | 2.67 | 1.145 | 0.252 | 4.5 |
| 139 | 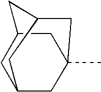 | 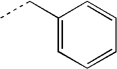 | S | 3.13 | 7.133 | 3.951 | 1.8 |
| 140 | 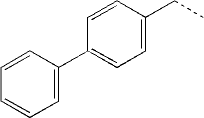 | 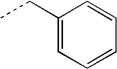 | S | 4.04 | 5.127 | 2.358 | 2.2 |
| 141 | 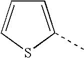 | 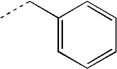 | S | 2.05 | 5.204 | 0.892 | 5.8 |
| 142 | 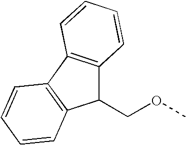 | 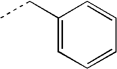 | S | 4.45 | 5.365 | 3.199 | 1.7 |
| 143 | 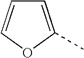 | 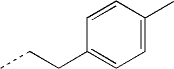 | rac | 1.3 | 1.3 ± 0.1 | 0.97 ± 0.08 | 1.3 |
| 144 | 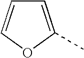 | 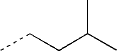 | rac | 1.0 | 4.4 ± 0.5 | 1.5 ± 0.2 | 2.9 |
| 145 | 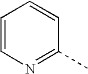 | 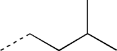 | rac | 1.0 | 12 ± 1 | 9 ± 2 | 1.3 |
| 146 | 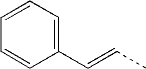 | 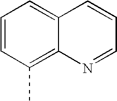 | S | 3.18 | 1.24 | 0.021 | 58.9 |
| 147 | 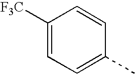 | 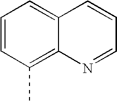 | S | 3.81 | 0.696 | 0.258 | 2.7 |
| 148 | 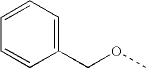 | 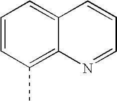 | S | 2.96 | 0.452 | 0.043 | 10.5 |
| 149 | 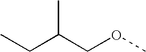 | 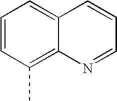 | S | 2.62 | 0.081 | 0.023 | 3.5 |
| 150 | 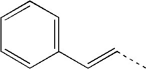 | 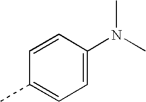 | S | 2.30 | 0.240 | 0.165 | 1.5 |
| 151 | 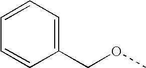 | 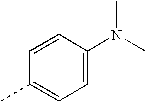 | S | 2.77 | 0.650 | 0.610 | 1.1 |
| 152 | 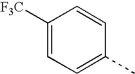 | 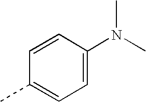 | S | 3.62 | 0.404 | 0.222 | 1.8 |
| 153 | 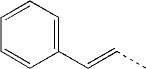 | 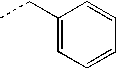 | S | 2.67 | 0.990 | 0.731 | 1.4 |
| 154 | 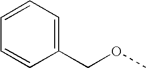 | 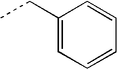 | S | 2.45 | 1.520 | 1.520 | 1 |
| 155 | 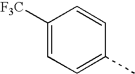 | 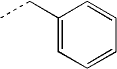 | S | 3.29 | 1.611 | 0.665 | 2.4 |
| 156 | 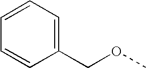 | 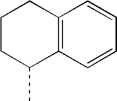 | S | 2.89 | 1.710 | 1.710 | 1 |
| 157 | 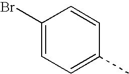 | 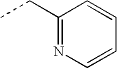 | S | 1.92 | 2.094 | 1.152 | 1.8 |
| 158 | 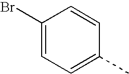 | 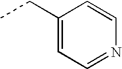 | S | 1.79 | 5.237 | 0.837 | 6.3 |
| 159 | 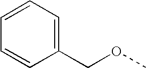 | 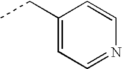 | S | 1.22 | 23.34 | 4.32 | 5.4 |
| 160 | 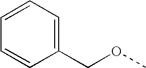 | 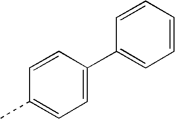 | S | 4.76 | 2.209 | 0.331 | 6.7 |
| 161 | 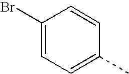 | 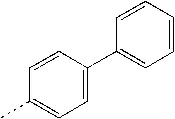 | S | 5.34 | 0.185 | 0.074 | 2.5 |
| 162 | 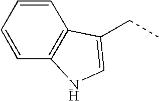 | 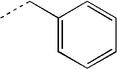 | S | 2.30 | >2.219 | 0.332 | >7 |
| 163 | 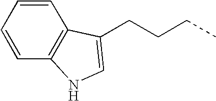 | 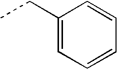 | S | 3.27 | 7.034 | 1.845 | 3.8 |
| 164 | 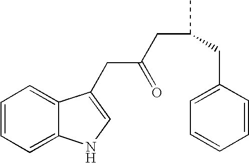 | 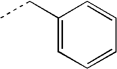 | S | 3.34 | 9.759 | NA | |
| 165 | 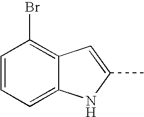 | 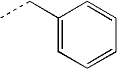 | S | 3.25 | 1.940 | 0.485 | 4.0 |
| 166 | 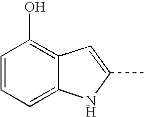 | 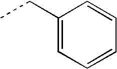 | S | 2.02 | 2.209 | 0.331 | 6.7 |
| 167 | 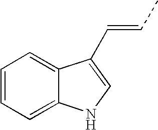 | 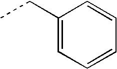 | S | 2.89 | 10.377 | 3.242 | 3.2 |
| 168 | 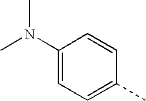 | 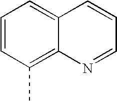 | S | | NA | NA | |

^{a}Selectivity Index = IC₅₀ (NFF)/IC₅₀ (MM96L).

A number of the more active compounds were also tested for cytotoxicity and cytoselectivity against six other human cancer cell lines two melanoma (SkMel28, DO4), prostate (DU145), breast (MCF-7), and ovarian (JAM, CI-80-13S). For comparison their results are also shown for MM96L and NFF cell lines. The results of these additional tests are given in table 9.

TABLE 10
Cytotoxicity of Selected Compounds for Various Cancer Cell
Lines
Cell line^{a} IC₅₀ (μM)

| Compound | NFF | MM96L | SkMel | DO4 | DU145 | MCF7 | JAM | C18013S |
|---|---|---|---|---|---|---|---|---|
| 22 | 0.35 (7) | 0.14 (9) | 3.0 (3) | 2.0 (3) | 0.61 (4) | 0.59 (5) | 1.24 (6) | 0.7 (2) |
| 24 | 0.83 (9) | 0.2 (1) | 5.7 (4) | 3.5 (2) | 3.8 (4) | 1.16 (2) | 2.0 (2) | 1.5 (3) |
| 40 | 0.8 (2) | 0.13 (9) | 1.7 (2) | 1.3 (4) | 0.4 (4) | 0.84 (9) | 0.75 (8) | 0.4 (3) |
| 44 | 0.8 (2) | 0.2 (1) | 2.5 (1) | 2.1 (3) | 1.70 (3) | 0.7 (2) | 1.8 (1) | 0.6 (5) |
| 59 | 0.60 | 0.10 | 1.09 | 1.05 | 0.33 | 0.39 | 0.43 | 0.39 |
| 61 | 0.32 | 0.17 | 1.11 | 0.83 | 0.37 | 0.48 | 0.60 | 0.38 |
| 67 | 2.20 | 0.20 | 2.59 | 2.73 | 1.06 | 0.96 | 1.47 | 1.24 |
| 70 | 8.20 | 0.90 | 6.14 | 7.12 | 3.76 | 3.30 | 7.22 | 2.50 |
| 78 | 3.60 | 0.51 | 2.33 | 2.48 | 1.06 | 0.97 | 2.30 | 1.36 |

TABLE 11
Antiproliferative Potencies of Compound 124.

| Cell Line^{a} | IC₅₀ (μM) | Selectivity^{b} |
|---|---|---|
| MM96L | 0.13 ± 0.02 | 6.7 |
| MM229 | 0.60 ± 0.08 | 1.5 |
| MM329 | 0.06 ± 0.04 | 15 |
| MM470 | 0.09 ± 0.07 | 10 |
| MM604 | 0.01 ± 0.01 | 87 |
| Mel RM | 0.95 ± 0.07 | 0.9 |
| Mel FH | 0.08 ± 0.04 | 11 |
| SK Mel 28 | 0.06 ± 0.04 | 15 |
| DO4 | 0.12 ± 0.03 | 7 |
| D14 | 0.26 ± 0.08 | 3 |
| D11 | 0.3 ± 0.2 | 3 |
| D17 | 0.06 ± 0.02 | 15 |
| LSP M2 | 0.45 ± 0.05 | 2 |
| AF-6 | 0.30 ± 0.03 | 3 |
| AO7 RM | 0.18 ± 0.07 | 5 |
| A2058 | 0.10 ± 0.02 | 9 |
| HeLa | 0.09 ± 0.01 | 10 |
| NFF | 0.87 ± 0.07 | 1 |

^{a}NFF, neonatal foreskin fibroblasts; MM96L, 229, 329, 470, 604, Mel RM and FH, SK-Mel-28, DO4, 11, 14, 17 melanoma; DU145, prostate; MCF-7 breast; JAM, Cl-80-13S, ovarian.
Standard deviations are in parentheses.
^{b}A more comprehensive list of non-melanoma cell lines to be added Selectivity Index = IC₅₀ (NFF)/IC₅₀(cancer cell line), recognizing that the IC50 for NFFs is an underestimate because many cells are selectively differentiated to a non-proliferating phenotype.

TABLE 12
Cytoselectivities (nM) For Six Antitumour Compounds (S and
R enantiomers) In Different Cancer Cell Lines^{a}

| Cancer | 126 | | 132 | | 146 | | 148 | | 149 | | 161 | |
|---|---|---|---|---|---|---|---|---|---|---|---|---|
| Cell | S | R | S | R | S | R | S | R | S | R | S | R |
| A549 | 148 | 191 | 69 | 121 | 73 | 196 | 248 | 104 | 291 | 453 | 335 | 18619 |
| DU145 | 61 | 52 | 22 | 33 | 30 | 63 | 131 | 39 | 130 | 221 | 149 | 18619 |
| HOP62 | 137 | 147 | 63 | 63 | 65 | 133 | 226 | 65 | 267 | 314 | 261 | 18619 |
| HT29 | 178 | 199 | 79 | 238 | 99 | 272 | 334 | 139 | 360 | 488 | 307 | 18619 |
| MCF-7 | 35 | 40 | 19 | 42 | 17 | 51 | 122 | 29 | 138 | 178 | 149 | 13033 |
| MM96L | 51 | 38 | 18 | 18 | 20 | 45 | 108 | 22 | 116 | 163 | 121 | 18619 |
| SK- | 73 | 57 | 33 | 43 | 37 | 87 | 146 | 43 | 170 | 267 | 168 | 18619 |
| MEL- | | | | | | | | | | | | |
| 28 | | | | | | | | | | | | |
| SK- | 32 | 44 | 18 | 23 | 29 | 66 | 70 | 20 | 100 | 115 | 102 | 18619 |
| MEL-5 | | | | | | | | | | | | |
| H520 | 63 | 188 | 26 | 180 | 27 | 152 | 118 | 46 | 129 | 337 | 117 | 18619 |
| T-47D | 45 | 63 | 19 | 42 | 20 | 99 | 93 | 51 | 198 | 291 | 197 | 18619 |
| CI80- | 65 | 43 | 20 | 27 | 28 | 91 | 124 | 29 | 149 | 203 | 158 | 18619 |
| 13S | | | | | | | | | | | | |
| JAM | 85 | 251 | 48 | 211 | 49 | 163 | 194 | 65 | 221 | 267 | 182 | 18619 |
| PC-3 | 220 | 387 | 148 | 148 | 109 | 543 | 528 | 269 | 651 | 2092 | 531 | 18619 |
| Col208 | 223 | —^{b} | 102 | — | 141 | — | 122 | — | 395 | — | 272 | — |

^{a}Colo208 (colon), DU145 (prostate), MCF-7 (breast), SK-MEL-28 (melanoma), A549 (lung), HOP62 (lung), HT29 (colon).
^{b}Not performed.

TABLE 13
Selectivity Index For Six Antitumour Compounds (S
enantiomer), Cancer Cell Compared to NFFs.

| Cancer Cell | 126 | 132 | 146 | 148 | 149 | 161 |
|---|---|---|---|---|---|---|
| A549 | 4 | 5 | 17 | 2 | 0.3 | 0.6 |
| DU145 | 9 | 15 | 41 | 3 | 0.6 | 1.2 |
| HOP62 | 4 | 5 | 19 | 2 | 0.3 | 0.7 |
| HT29 | 3 | 4 | 13 | 1.4 | 0.2 | 0.6 |
| MCF-7 | 16 | 18 | 71 | 4 | 0.6 | 1.2 |
| MM96L | 11 | 19 | 63 | 4 | 0.7 | 1.5 |
| SK-MEL-28 | 8 | 10 | 34 | 3 | 0.5 | 1.1 |
| SK-MEL-5 | 18 | 18 | 43 | 6 | 0.8 | 1.8 |
| H520 | 9 | 13 | 46 | 4 | 0.6 | 1.6 |
| T-47D | 13 | 18 | 63 | 5 | 0.4 | 0.9 |
| CI80-13S | 9 | 17 | 44 | 4 | 0.5 | 1.2 |
| JAM | 7 | 7 | 25 | 2 | 0.4 | 1.0 |
| PC-3 | 3 | 2 | 11 | 0.9 | 0.1 | 0.3 |
| Col208 | 1.5 | 3 | 9 | 4 | 0.2 | 0.6 |

Selectivity Index = IC₅₀(NFF)/IC₅₀(cancer cell line), recognizing that the IC50 for NFFs is an underestimate because many cells are selectively differentiated to a non-proliferating phenotype.

Histone Hyperacetylation. The more potent compounds were tested for inhibition of histone deacetylase by monitoring the acetylation state of histone H4 using Triton-acetic acid-urea gel electrophoresis.

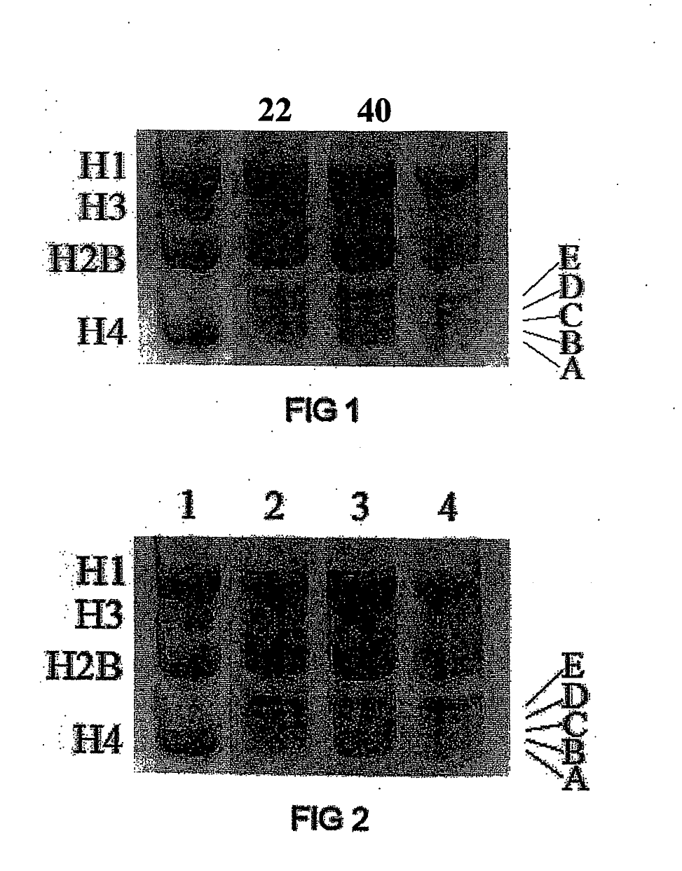

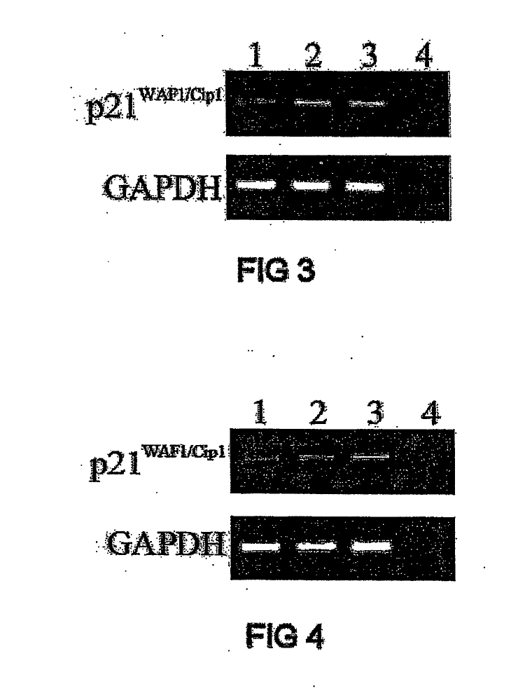

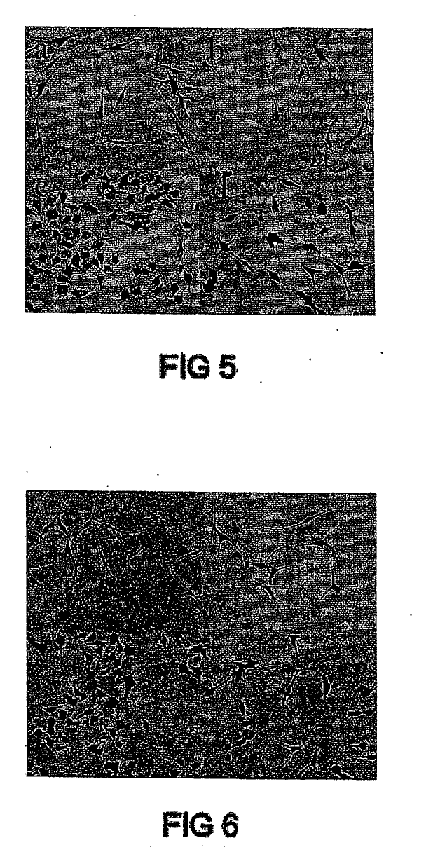

One set of results is shown in FIG. 1 for the compounds of examples 22 and 40, showing hyperacetylation of H4. It was not necessary to quantitate histone deacetylation because the compounds inhibit HDAC activity in both normal and cancer cells and has no impact on the cytoselectivity. The known HDAC inhibitor, TSA, included for comparison, showed similar levels of hyperacetylation indicated by the mobility shift of histone H4. Clearly visible in untreated cells is the non-acetylated histone H4 (lane 1, arrow A). In the extracts from cells treated with 10 μg/ml of 22 and 40, histone H4 was observed in a variety of acetylation states, ranging from non-acetylated to tetra-acetylated. These results support the notion that this compound series inhibits HDACs.

Further results are outlined in FIG. 2 for just compounds 40 and 73, showing hyperacetylation of H4. Once again, it was not necessary to quantitate histone deacetylation because the compounds inhibit HDAC activity in both normal and cancer cells and has no impact on the cytoselectivity. The known HDAC inhibitor, TSA, included for comparison, showed similar levels of hyperacetylation indicated by the mobility shift of histone H4. Clearly visible in untreated cells in the non-acetylated histone H4 (lane 1, arrow A). In the extracts from cells treated with 10 μg/ml of 40 and 73, histone H4 was observed in a variety of acetylation states, ranging from non-acetylated to tetra-acetylated. These results support the notion that this compound series inhibits HDACs.

Induction of p21 Expression. It has been postulated that histone acetylation is associated with activation of gene transcription. It has been shown that the action of HDAC inhibitors on gene expression is somewhat selective, and does not lead to global deregulation of transcription as may be expected. In cells cultured with TSA, the expression of only 2% of genes was significantly altered, indicating a remarkable specificity. Possibly the best characterised gene to be induced following exposure to different HDAC inhibitors is that of the cyclin-dependent kinase inhibitor p21^{WAF1/Cip1}, which blocks cyclin-dependent kinase activity thereby causing cell-cycle arrest in G1. HDAC inhibitors are thought to act directly on the CDKN1A promoter rather than an upstream target. The HDAC inhibitor SAHA induces accumulation of acetylated histones in the chromatin associated with the CDKN1A gene, and this correlates with the observed increase in transcription. Sp-1 transcription factor binding sites in the promoter of CDKN1A are considered to be crucial for the observed induction, and for a number of other targets. The capacity of novel compounds to induce expression of the cyclin-dependent kinase inhibitor p21^{WAF1/Cip1} (CDKN1A) was examined by semi-quantitative RT-PCR after 8/24 hours of treatment in MM96L and NFF cell types.

Cell Treatment and Total RNA Isolation. Cells were seeded in 25 cm² flasks in 10% heat-inactivated foetal calf serum (CSL, Melbourne, Australia) in RPMI 1640 medium supplemented with 100 U/mL penicillin, 100 μg/mL streptomycin, 3 mM HEPES, and incubated at 5% CO₂, 99% humidity at 37° C. for 16 hours before treatment. Cells were treated with 10 μg/mL of drug and RNA harvested at the indicated times following treatment. Total RNA was extracted from cells using the Qiagen RNeasy Kit as per manufacturer's instructions. RNA was analysed for sufficient quality by formamide agarose gel electrophoresis, and quantified by spectrophotometry.

p21 Expression. The semi-quantitative analysis of mRNA expression of p21^{WAF1/Cip1} was carried out by RT-PCR. First strand synthesis was performed using 2 μg total RNA with 0.5 μg oligo (dT)₁₅ and 200 U SuperScript II (Invitrogen, Carlsbad, Calif.), at 42° C. for 50 minutes in a final volume of 20 μL. Polymerase chain reaction was performed using 10 μL of a 1 in 10 dilution of the first strand cDNA, under standard conditions with the polymerase DyNAzyme (Finnzymes, Melbourne, Australia). Oligonucleotide primers and conditions used in the PCR were as follows: p21^{WAF1/Cip1} F 5′-ATT AGC AGC GGA ACA AGG AGT CAG ACA T-3′, p21^{WAF1/Cip1} R 5′-CTG TGA AAG ACA CAG AAC AGT ACA GGG T-3′ with initial denaturation at 94° C. for 7 mins, 27 cycles of 94° C. for 45 s, 60° C. for 40 s and 72° C. for 60 s, with the final extension for 5 minutes; GAPDH F 5′-GGC TCT CCA GAA CAT CAT CCC TGC-3′, GAPDH R 5′-GGG TGT CGC TGT TGA AGT CAG AGG-3′ with initial denaturation at 94° C. for 7 minutes, 25 cycles of 94° C. for 45 s, 62° C. for 40 s and 72° C. for 60 s, with the final extension for 5 minutes. Products were analysed by agarose gel electrophoresis, and visualised on a UV light box. Product intensity was determined to increase linearly with number of cycles and amount of mRNA used, by densitometric analysis using ImageQuaNT 4.2 software (Molecular Dynamics, Sunnyvale, Calif.). Quantitation of p21^{WAF1/Cip1} induction was also performed by densitometric analysis using ImageQuaNT 4.2 software following normalisation to GAPDH product intensity.

Morphological Reversion. Cells were plated into 96-well microtitre plates at 5×10³ cells/well, and allowed to adhere overnight. Compounds were added to culture medium at the indicated concentrations, and plates incubated in the above conditions for 24 hours. Cells were then washed once with Hank's Balanced Salt Solution (HBSS; Gibco/Invitrogen, Grand Island, N.Y.), and fixed in 4% buffered formalin for 1 hour at room temperature. The fixed cells were then washed once further with HBSS and stained with 1% Crystal Violet in methanol for 5 minutes. Excess stain was removed by washing with tap water, before the microtitre plate being air dried at 37° C. Photographs were taken using a Leica DMIRB inverted microscope.

Oral Bioavailability

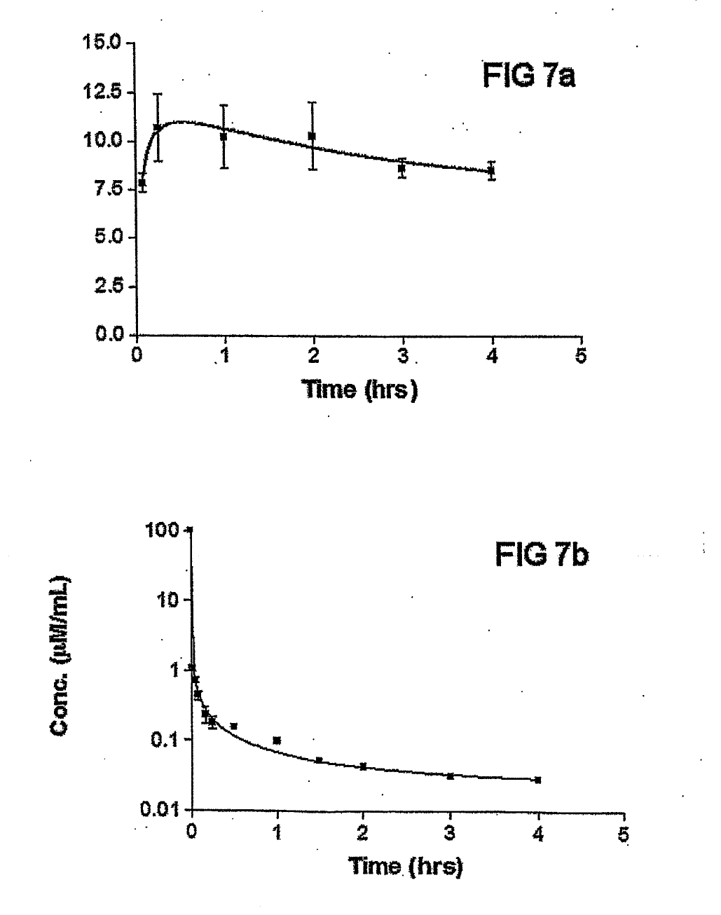

Still the most effective form of drug delivery, in terms of ease of administration, probability of patient compliance, and systemic penetration, is the oral route which is the preferred form of delivery of antitumor drugs. Compounds of the invention were examined to see whether this new series had the expected favorable properties for oral delivery. The results indicate that the compound series being developed here is, in general, orally bioavailable. Compound 24 (Log D_{7.0} 2.7) was administered intravenously and orally to rats. When delivered at 5 mg/kg in 4:1 olive oil:DMSO to three rats starved prior to dosing, high serum levels of drug were maintained (FIG. 7), with Cmax˜6 μg/mL for >4 h examined in this preliminary study and Tmax˜15 min. Neither vehicle nor fasting had any significant effects on these parameters.

Finally, it will be appreciated that there may be other variations and modifications to the methods described herein that are also within the scope of the present invention.

### REFERENCES

Marks, P. A., Richon, V. M., Kiyokawa, H., Rifkind, R. A. Inducing differentiation of transformed cells with hybrid polar compounds: a cell cycle-dependent process. PNAS (USA) 1994, 91, 10251-4
Rifkind, R. A., Richon, V. M., Marks, P. A. Induced differentiation, the cell cycle and the treatment of cancer. Pharmacol. Therap. 1996, 69, 97-102.
Leszczyniecka, M., Roberts, T., Dent, P., Grant, S., Fisher P. B.; Differentiation therapy of human cancer: basic-science and clinical implications. Pharmacol. Therap. 2001, 90, 105-156.
Tsuji, N., Kobayashi, M., Nagashima, K., Wakisaka, Y., Koizumi, K. A new antifungal antibiotic, trichostatin. J. Antibiot. 1976 29, 1-6.
Yoshida, M.; Kijima, M.; Akita, M.; Beppu, T. Potent and specifik Inhibition of Mammalian Histone Deacetylase Both in Vivo and in Vitro by Trichostatin A. J. Biol. Chem. 1990, 265, 17174-17179.
Cress, W. D.; Seto, E. Histone Deacetylase, Transcriptional Control, and Cancer. J. Cell. Physiol. 2000, 184, 1-16.
Marks, P. A. Richon V M, Breslow R, Rifkind R A. Histone deacetylase inhibitors as new cancer drugs. Curr. Opin. Oncol. 2001, 13, 477-483.
Kijima, M.; Yoshida, M.; Sugita, K.; Horinouchi, S.; Beppu, T. Trapoxin, an Antitumor Cyclic Tetrapeptide, Is an Irreverible Inhibitor of Mammalian Histone Deacetylase. J. Biol. Chem. 1993, 268, 22429-22435.
Vigushin, D. M.; Coombes, R. C. Histone deacetylase inhibitors in cancer treatment. Anti-Cancer Drugs. 2002, 13, 1-13.
Furumai, R.; Komatsu, Y.; Nishino, N.; Khochbin, S.: Yoshida, M.; Horinouchi, S. Potent histone deacetylase inhibitors built from trichostatin A and cyclic tetrapeptide antibiotics including trapoxin. PNAS. 2001, 98, 87-92.
Parsons, P. G., Bowman, E. P. W. and Blakely, R. L. Selective toxicity of deoxyadenosine analogues in human melanoma cell lines. Biochem. Pharmacol. 1986, 35, 4025-4029.
Todaro, G. J., Fryling, C. and De Larco, J. E. Transforming growth factors produced by certain human tumour cells: polypeptides that interact with epidermal growth factor receptors. Proc. Natl. Acad. Sci. U.S.A. 1980, 77, 5258-5262.
Glenn, M. P.; Kahnberg, P.; Boyle, G. M.; Hansford, K. A.; Hans, D.; Martyn, A. C.; Parsons, G. P.; Fairlie, D. P. Anti-Proliferative And Phenotype-Transforming Antitumor Agents Derived From Cysteine J. Med. Chem. 2004, 47, 2984-2994.

1-83. (canceled)

84. A compound having the formula (I), or a pharmaceutically acceptable derivative, salt, racemate, isomer or tautomer thereof: 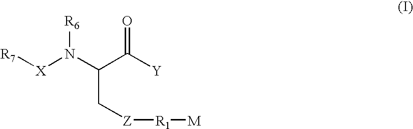
wherein
Z is S or CH₂;
R₁ is a linking moiety;
M is a zinc binding moiety containing at least one heteroatom;
R₆ is selected from the group consisting of H, optionally substituted alkyl, optionally substituted alkenyl, optionally substituted alkynyl and a nitrogen protecting group;
X is selected from the group consisting of: 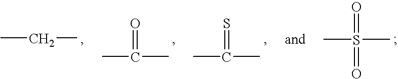
Y is selected from the group consisting: of —NR₄R₅, —OR₄, —SR₄, —CH₂R₄, CHR₄R₅, C(R₄)₂R₅, PH and PR₄R₅,
wherein R₄ is a group of formula: 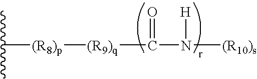
wherein R₈, R₉ and R₁₀ are each independently selected from the group consisting of optionally substituted alkyl, optionally substituted alkenyl, optionally substituted alkynyl, optionally substituted cycloalkyl, optionally substituted aryl, optionally substituted heteroaryl, and optionally substituted heterocycloalkyl;
p, q, r and s are each independently 0 or 1, provided that at least one of p, q or s is 1;
R₅ is H or a group of formula: 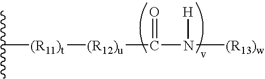
wherein R₁₁, R₁₂ and R₁₁₃ are each independently selected from the group consisting of optionally substituted alkyl, optionally substituted alkenyl, optionally substituted alkynyl, optionally substituted cycloalkyl, optionally substituted aryl, optionally substituted heteroaryl, or optionally substituted heterocycloalkyl;
t, u, v and w are each independently 0 or 1, provided that at least one of t, u and w is 1;
R₇ is a group of formula:
(R₁₆)_{z}—(R₁₅)_{y}—(R₁₄)ₓ—
wherein R₁₄, R₁₅ and R₁₆ are independently selected from the group consisting of optionally substituted alkyl, optionally substituted alkenyl, optionally substituted alkynyl, optionally substituted cycloalkyl, optionally substituted aryl, optionally substituted heteroaryl and optionally substituted heterocycloalkyl,
x, y and z are independently 0 and 1 with the proviso that at least one of x, y and z is 1,
with the proviso that:
when Z is CH₂ and Y is 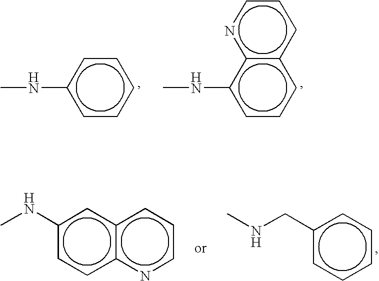
then R₆ is not H, X is not 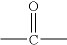
and R₇ is not 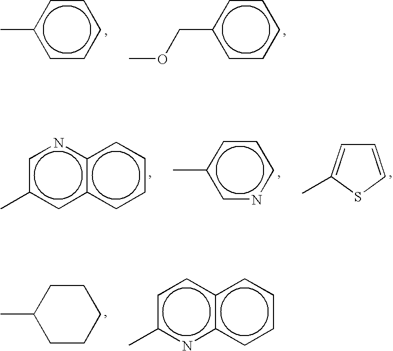
or OC(CH₃)₃; and
when Z is CH₂ and Y is 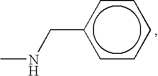
then R₆ is not H, X is not 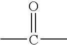
and R₇ is not —CH₃.

85. A compound as in claim 84, wherein the zinc binding moiety is a group of formula —C(O)—NR₂—OR₃ where R₂ is H, optionally substituted alkyl optionally substituted alkenyl, optionally substituted alkynyl, optionally substituted aryl or a nitrogen protecting group and R₃ is H, optionally substituted alkyl, optionally substituted alkenyl, optionally substituted alkynyl, optionally substituted aryl or an oxygen protecting group.

86. A compound as in claim 85, wherein the linking moiety has between 1 and 9 atoms in the normal chain.

87. A compound as in claim 86, wherein the linking moiety is an n-propyl chain.

88. A compound having the formula (IIIa), or a pharmaceutically acceptable derivative, salt, racemate, isomer or tautomer thereof: 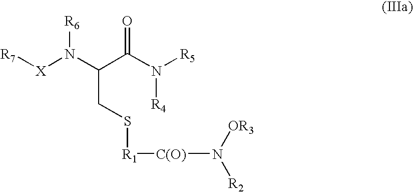
wherein
R₁ is optionally substituted C₁-C₄ alkyl, optionally substituted C₁-C₄ alkenyl or optionally substituted C₁-C₄ alkynyl;
R₂ is H, optionally substituted alkyl, optionally substituted alkenyl, optionally substituted alkynyl, optionally substituted aryl, or a nitrogen protecting group;
R₃ is H, optionally substituted alkyl, optionally substituted alkenyl, optionally substituted alkynyl, optionally substituted aryl or an oxygen protecting group;
R₄ is a group of formula: 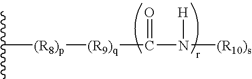
wherein R₈, R₉ and R₁₀ are each independently selected from the group consisting of optionally substituted alkyl, optionally substituted alkenyl, optionally substituted alkynyl, optionally substituted cycloalkyl, optionally substituted aryl, optionally substituted heteroaryl, and optionally substituted heterocycloalkyl;
p, q, r and s are each independently 0 or 1, provided that at least one of p, q or s is 1;
R₅ is H or a group of formula: 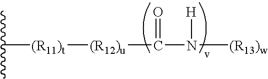
wherein R₁₁, R₁₂ and R₁₁₃ are each independently selected from the group consisting of optionally substituted alkyl, optionally substituted alkenyl, optionally substituted alkynyl, optionally substituted cycloalkyl, optionally substituted aryl, optionally substituted heteroaryl, or optionally substituted heterocycloalkyl;
t, u, v and w are each independently 0 or 1, provided that at least one of t, u and w is 1.
R₆ is selected from the group consisting of H, optionally substituted alkyl, optionally substituted alkenyl, optionally substituted alkynyl and a nitrogen protecting group;
X is selected from the group consisting of 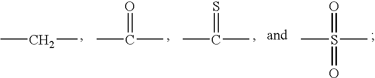
R₇ is a group of formula:
(R₁₆)_{z}—(R₁₅)_{y}—(R₁₄)ₓ—
wherein R₁₄, R₁₅ and R₁₆ are independently selected from the group consisting of optionally substituted alkyl, optionally substituted alkenyl, optionally substituted alkynyl, optionally substituted cycloalkyl, optionally substituted aryl, optionally substituted heteroaryl and optionally substituted heterocycloalkyl;
x, y and z are independently 0 and 1 with the proviso that at least one of x, y and z is 1.

89. A compound as in claim 88, wherein R₁ is n-propyl.

90. A compound as in claim 88, wherein R₂ is either H, optionally substituted C₁-C₄ alkyl or a nitrogen protecting group.

91. A compound as in claim 88, wherein R₃ is either H, optionally substituted C₁-C₄ alkyl or an oxygen protecting group.

92. A compound as in claim 88, wherein R₄ is of the formula: 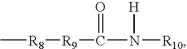
wherein R₈, R₉ and R₁₀ are each independently selected from the group consisting of optionally substituted alkyl, optionally substituted alkenyl, optionally substituted alkynyl, optionally substituted cycloalkyl optionally substituted aryl, optionally substituted heteroaryl, and optionally substituted heterocycloalkyl.

93. A compound as in claim 92, wherein R₄ is a group of the formula. 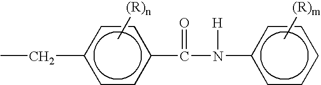
wherein each R is independently selected from the group consisting of alkyl, alkenyl, alkynyl aryl, heteroaryl, cycloalkyl, heterocycloalkyl, halo, haloalkyl, haloalkenyl, haloalkynyl, haloaryl, haloheteroaryl, halocycloalkyl, haloheterocycloalkyl hydroxy, alkoxy, alkenyloxy, aryloxy, heteroaryloxy, cycloalkyloxy, heterocycloalkyloxy, benzyloxy, haloalkoxy, haloalkenyloxy, haloaryloxy, halohetoraryloxy, nitro, nitroalkyl, nitroalkenyl, nitroalkynyl, nitroaryl, nitroheteroaryl, nitroheterocyclyoalkyl, amino, alkylamino, dialkylamino, alkenylamino, alkynylamino, arylamino, heteroarylamino, diarylamino, benzylamino, dibenzylamino, acyl, alkenylacyl, alkynylacyl, arylacyl, heteroarylacyl, acylamino, diacylamino, acyloxy, alkylsulphonyloxy, arylsulphonyloxy, heterocycloalkylamino, alkylsulphonyl, arylsulphonyl, carboalkoxy, carboaryloxy, alkylthio, benzylthio, acylthio, cyano, nitro, sulfate and phosphate;
n is 0-4, and
m is 0-5.

94. A compound as in claim 92, wherein R₄ has one of the following formulae: 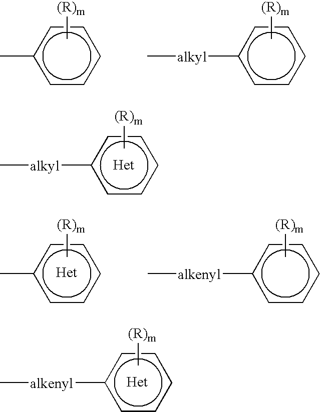
wherein each R is independently selected from the group consisting of alkyl, alkenyl, alkynyl, aryl, heteroaryl, cycloalkyl, heterocycloalkyl, halo, haloalkyl, haloalkenyl, haloalkynyl, haloaryl, haloheteroaryl, halocycloalkyl, haloheterocycloalkyl, hydroxy, alkoxy, alkenyloxy, aryloxy, heteroaryloxy, cycloalkyloxy, heterocycloalkyloxy, benzyloxy, haloalkoxy, haloalkenyloxy, haloaryloxy, halohetoraryloxy, nitro, nitroalkyl, nitroalkenyl nitroalkynyl, nitroaryl, nitroheteroaryl, nitroheterocyclyoalkyl, amino, alkylamino, dialkylamino, alkenylamino, alkynylamino, arylamino, heteroarylamino, diarylamino, benzylamino, dibenzylamino, acyl, alkenylacyl, alkynylacyl, arylacyl, heteroarylacyl, acylamino, diacylamino, acyloxy, alklysulphonlyoxy, arylsulphonyloxy, heterocycloalkylamino, alkylsulphonyl, arylsulphnyl, carboalkoxy, carboaryloxy, alkylthio, benzylthio, acylthio, cyano, nitro, sulfate and phosphate;
and each m is from 0-5.

95. A compound as in claim 88, wherein R₅ is either H or optionally substituted alkyl.

96. A compound as in claim 88, wherein X is a carbonyl group.

97. A compound as in claim 96, wherein R₆ is either H or a nitrogen protecting group.

98. A compound as in claim 96, wherein R₇ is selected from the group consisting of optionally substituted alkyl, optionally substituted alkenyl, optionally substituted alkynyl, optionally substituted aryl, optionally substituted cycloalkyl, optionally substituted heteroaryl, optionally substituted heterocycloalkyl, optionally substituted aryl alkyl, optionally substituted heteroaryl alkyl, optionally substituted cycloalkyl alkyl, optionally substituted heterocycloalkyl alkyl, optionally substituted aryl alkenyl, optionally substituted hetero alkenyl, optionally substituted cycloalkyl alkenyl, optionally substituted heterocycloalkyl alkenyl, optionally substituted aryl alkynyl, optionally substituted heteroaryl alkynyl, optionally substituted cycloalkyl alkynyl, and optionally substituted heterocycloalkyl alkynyl.

99. A compound as in claim 88, wherein the compound has a potency of cytotoxicity of IC₅₀≦10 μM against MM96 melanoma cells.

100. A compound as in claim 99, wherein the compound has a Selectivity Index of ≧1.5.

101. A compound as in claim 100, wherein the compound has a potency of IC₅₀≦1 μM against the MM96 melanoma cells and a Selectivity Index of ≧3.

102. A compound as in claim 101, wherein the compound has a potency of IC₅₀≦0.5 μM against the MM96 melanoma cells and a Selectivity Index of ≧4.

103. A compound as in claim 84, wherein the compound has the formula (IIIb): 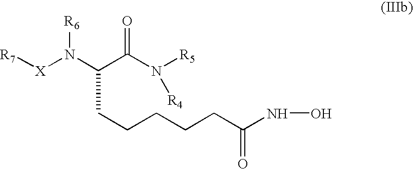
wherein
R₄ is a group of formula: 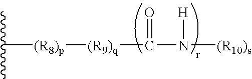
wherein R₈, R₉ and R₁₀ are each independently selected from the group consisting of optionally substituted alkyl, optionally substituted alkenyl, optionally substituted alkynyl, optionally substituted cycloalkyl, optionally substituted aryl, optionally substituted heteroaryl, and optionally substituted heterocycloalkyl;
p, q, r and s are each independently 0 or 1, provided that at least one of p, q or s is 1;
R₅ is H or a group of formula: 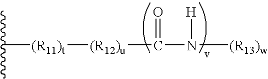
wherein R₁₁, R₁₂ and R₁₁₃ are each independently selected from the group consisting of optionally substituted alkyl, optionally substituted alkenyl, optionally substituted alkynyl, optionally substituted cycloalkyl, optionally substituted aryl, optionally substituted heteroaryl, or optionally substituted heterocycloalkyl;
t, u, v and w are each independently 0 or 1, provided that at least one of t, u and w is 1.
R₆ is selected from the group consisting of H, optionally substituted alkyl, optionally substituted alkenyl, optionally substituted alkynyl and a nitrogen protecting group;
X is selected from the group consisting of 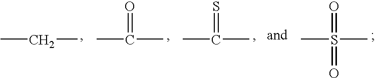
R₇ is a group of formula:
(R₁₆)_{z}—(R₁₅)_{y}—(R₁₄)ₓ—
wherein R₁₄, R₁₅ and R₁₆ are independently selected from the group consisting of optionally substituted alkyl, optionally substituted alkenyl, optionally substituted alkynyl, optionally substituted cycloalkyl, optionally substituted aryl, optionally substituted heteroaryl and optionally substituted heterocycloalkyl;
x, y and z are independently 0 and 1 with the proviso that at least one of x, y and z is 1.

104. A compound as in claim 103, wherein R₄ is of the formula: 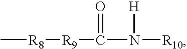
wherein R₈, R₉ and R₁₀ are each independently selected from the group consisting of optionally substituted alkyl, optionally substituted alkenyl, optionally substituted alkynyl, optionally substituted cycloalkyl, optionally substituted aryl, optionally substituted heteroaryl, and optionally substituted heterocycloalkyl.

105. A compound as in claim 104, wherein R₄ is a group of the formula. 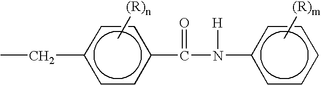
wherein each R is independently selected from the group consisting of alkyl, alkenyl, alkynyl, aryl, heteroaryl, cycloalkyl, heterocycloalkyl, halo, haloalkyl, haloalkenyl, haloalkynyl, haloaryl, haloheteroaryl, halocycloalkyl haloheterocycloalkyl, hydroxy, alkoxy, alkenyloxy, aryloxy, heteroaryloxy, cycloalkyloxy, heterocycloalkyloxy, benzyloxy, haloalkoxy, haloalkenyloxy, haloaryloxy, halohetoraryloxy, nitro, nitroalkyl, nitroalkenyl, nitroalkynyl, nitroaryl, nitroheteroaryl, nitroheterocyclyoalkyl, amino, alkylamino, dialkylamino, alkenylamino, alkynylamino, arylamino, heteroarylamino, diarylamino, benzylamino, dibenzylamino, acyl, alkenylacyl, alkynylacyl, arylacyl, heteroarylacyl, acylamino, diacylamino, acyloxy, alkylsulphonyloxy, arylsulphonyloxy, heterocycloalkylamino, alkylsulphonyl, arylsulphonyl, carboalkoxy, carboaryloxy, alkylthio, benzylthio, acylthio, cyano, nitro, sulfate and phosphate;
n is 0-4, and
m is 0-5.

106. A compound as in claim 103, wherein R₄ has one of the following formulae: 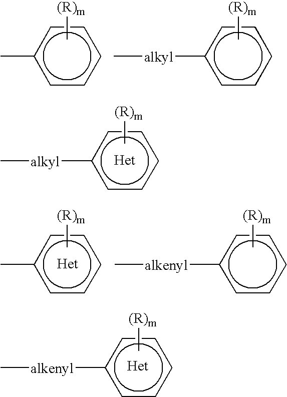
wherein each R is independently selected from the group consisting of alkyl, alkenyl, alkynyl, aryl, heteroaryl, cycloalkyl, heterocycloalkyl, halo, haloalkyl, haloalkenyl, haloalkynyl, haloaryl,

111. A compound as in claim 103, wherein the compound has a potency of cytotoxicity of IC₅₀≦10 μM against MM96 melanoma cells.

112. A compound as in claim 111, wherein the compound has a Selectivity Index of ≧1.5.

113. A compound as in claim 112, wherein the compound has a potency of IC₅₀≦1 μM against the MM96 melanoma cells and a Selectivity Index of ≧3.

114. A compound as in claim 113, wherein the compound has a potency of IC₅₀≦0.5 μM against the MM96 melanoma cells and a Selectivity Index of ≧4.

115. A method for the treatment of cancer in an animal, the method including the step of administering to the animal in need of such treatment an effective amount of a compound having the formula (I), or a pharmaceutically acceptable derivative, salt, racemate, isomer or tautomer thereof: 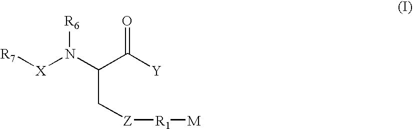
wherein
Z is S or —CH₂—;
R₁ is a linking moiety;
haloheteroaryl, halocycloalkyl, haloheterocycloalkyl hydroxy, alkoxy, alkenyloxy, aryloxy, heteroaryloxy, cycloalkyloxy, heterocycloalkyloxy, benzyloxy, haloalkoxy, haloalkenyloxy, haloaryloxy, halohetoraryloxy, nitro, nitroalkyl, nitroalkenyl nitroalkynyl, nitroaryl, nitroheteroaryl, nitroheterocyclyoalkyl, amino, alkylamino, dialkylamino, alkenylamino, alkynylamino, arylamino, heteroarylamino, diarylamino, benzylamino, dibenzylamino, acyl, alkenylacyl, alkynylacyl, arylacyl, heteroarylacyl, acylamino, diacylamino, acyloxy, alklysulphonlyoxy, arylsulphonyloxy, heterocycloalkylamino, alkylsulphonyl, arylsulphnyl, carboalkoxy, carboaryloxy, alkylthio, benzylthio, acylthio, cyano, nitro, sulfate and phosphate;
and each m is from 0-5.

107. A compound as in claim 103, wherein R₅ is H.

108. A compound as in claim 103, wherein X is a carbonyl group.

109. A compound as in claim 108, wherein R₆ is either H or a nitrogen protecting group.

110. A compound as in claim 108, wherein R₇ is selected from the group consisting of optionally substituted alkyl, optionally substituted alkenyl, optionally substituted alkynyl, optionally substituted aryl, optionally substituted cycloalkyl, optionally substituted heteroaryl, optionally substituted heterocycloalkyl, optionally substituted aryl alkyl, optionally substituted heteroaryl alkyl, optionally substituted cycloalkyl alkyl optionally substituted heterocycloalkyl alkyl, optionally substituted aryl alkenyl, optionally substituted hetero alkenyl, optionally substituted cycloalkyl alkenyl, optionally substituted heterocycloalkyl alkenyl, optionally substituted aryl alkynyl, optionally substituted heteroaryl alkynyl, optionally substituted cycloalkyl alkynyl, and optionally substituted heterocycloalkyl alkynyl.
M is a zinc binding moiety containing at least one heteroatom;
R₆ is selected from the group consisting of H, optionally substituted alkyl, optionally substituted alkenyl, optionally substituted alkynyl and a nitrogen protecting group;
X is selected from the group consisting of: 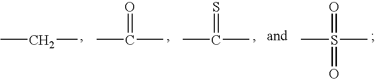
Y is selected from the group consisting: of —NR₄R₅, —OR₄, —SR₄, —CH₂R₄, CHR₅, C(R₄)₂R₅, PHR₄ and PR₄R₅,
wherein R₄ is a group of formula: 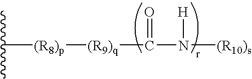
wherein R₈, R₉ and R₁₀ are each independently selected from the group consisting of optionally substituted alkyl, optionally substituted alkenyl, optionally substituted alkynyl, optionally substituted cycloalkyl, optionally substituted aryl, optionally substituted heteroaryl, and optionally substituted heterocycloalkyl;
p, q, r and s are each independently 0 or 1, provided that at least one of p, q or s is 1;
R₅ is H or a group of formula: 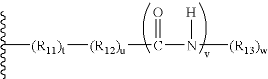
wherein R₁₁, R₁₂ and R₁₁₃ are each independently selected from the group consisting of optionally substituted alkyl, optionally substituted alkenyl, optionally substituted alkynyl, optionally substituted cycloalkyl, optionally substituted aryl optionally substituted heteroaryl, or optionally substituted heterocycloalkyl;
t, u, v and w are each independently 0 or 1, provided that at least one of t, u and w is 1;
R₇ is a group of formula:
(R₁₆)_{z}—(R₁₅)_{y}—(R₁₄)ₓ—
wherein R₁₄, R₁₅ and R₁₆ are independently selected from the group consisting of optionally substituted alkyl, optionally substituted alkenyl, optionally substituted alkynyl, optionally substituted cycloalkyl, optionally substituted aryl, optionally substituted heteroaryl and optionally substituted heterocycloalkyl,
x, y and z are independently 0 and 1 with the proviso that at least one of x, y and z is 1,
with the proviso that:
when Z is CH₂ and Y is 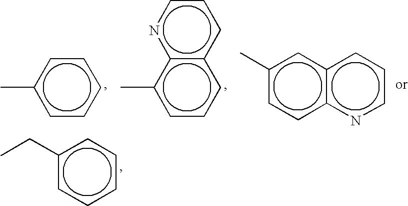
then R₆ is not H, X is not 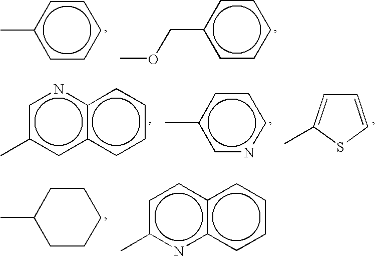
and R₇ is not 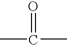
or OC(CH₃)₃; and
when Z is CH₂ and Y is 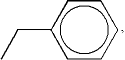
then R₆ is not H, X is not 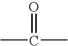
and R₇ is not —CH₃.

116. A method as in claim 115, wherein the linking moiety has between 1 and 9 atoms in the normal chain.

117. A method as in claim 116, wherein the linking moiety is an n-propyl chain.

118. A method as in claim 115, wherein Z is S.

119. A method as in claim 115, wherein the compound has the formula (III): 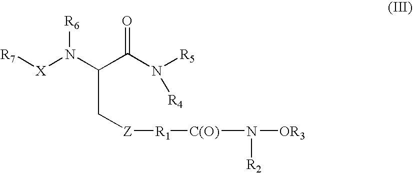
wherein
Z is S or CH₂;
R₁ is optionally substituted C₁-C₄ alkyl, optionally substituted C₁-C₄ alkenyl or optionally substituted C₁-C₄ alkynyl;
R₂ is H, optionally substituted alkyl, optionally substituted alkenyl, optionally substituted alkynyl, optionally substituted aryl, or a nitrogen protecting group;
R₃ is H, optionally substituted alkyl, optionally substituted alkenyl, optionally substituted alkynyl, optionally substituted aryl or an oxygen protecting group;
R₄ is a group of formula: 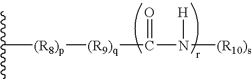
wherein R₈, R₉ and R₁₀ are each independently selected from the group consisting of optionally substituted alkyl, optionally substituted alkenyl, optionally substituted alkynyl, optionally substituted cycloalkyl, optionally substituted aryl, optionally substituted heteroaryl, and optionally substituted heterocycloalkyl;
p, q, r and s are each independently 0 or 1, provided that at least one of p, q or s is 1;
R₅ is H or a group of formula: 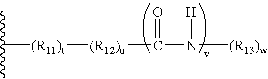
wherein R₁₁, R₁₂ and R₁₁₃ are each independently selected from the group consisting of optionally substituted alkyl, optionally substituted alkenyl, optionally substituted alkynyl, optionally substituted cycloalkyl, optionally substituted aryl, optionally substituted heteroaryl, or optionally substituted heterocycloalkyl;
t, u, v and w are each independently 0 or 1, provided that at least one of t, u and w is 1;
R₆ is selected from the group consisting of H, optionally substituted alkyl, optionally substituted alkenyl, optionally substituted alkynyl and a nitrogen protecting group;
X is selected from the group consisting of 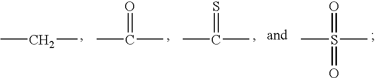
R₇ is a group of formula:
(R₁₆)_{z}—(R₁₅)_{y}—(R₁₄)ₓ—
wherein R₁₄, R₁₅ and R₁₆ are independently selected from the group consisting of optionally substituted alkyl, optionally substituted alkenyl, optionally substituted alkynyl, optionally substituted cycloalkyl, optionally substituted aryl, optionally substituted heteroaryl and optionally substituted heterocycloalkyl,
x, y and z are independently 0 and 1 with the proviso that at least one of x, y and z is 1.

120. A method for the treatment of cancer as in claim 119, wherein R₁ is optionally substituted C₁-C₄ alkyl.

121. A method for the treatment of cancer as in claim 120, wherein R₁ is propyl.

122. A method for the treatment of cancer as in claim 119, wherein Z is S.

123. A method for the treatment of cancer as in claim 119, wherein R₂ is either H, optionally substituted C₁-C₄ alkyl or a nitrogen protecting group.

124. A method for the treatment of cancer as in claim 119, wherein R₃ is either H, optionally substituted C₁-C₄ alkyl or an oxygen protecting group.

125. A method for the treatment of cancer as in claim 119, wherein R₄ is of the formula: 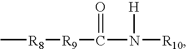
wherein R₈, R₉ and R₁₀ are each independently selected from the group consisting of optionally substituted alkyl optionally substituted alkenyl, optionally substituted alkynyl, optionally substituted cycloalkyl, optionally substituted aryl optionally substituted heteroaryl, and optionally substituted heterocycloalkyl.

126. A method for the treatment of cancer as in claim 125, wherein R₄ is a group of the formula. 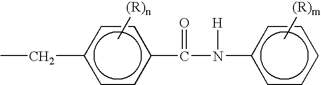
wherein each R is independently selected from the group consisting of alkyl, alkenyl, alkynyl, aryl, heteroaryl, cycloalkyl, heterocycloalkyl, halo, haloalkyl, haloalkenyl, haloalkynyl, haloaryl, haloheteroaryl, halocycloalkyl, haloheterocycloalkyl, hydroxy, alkoxy, alkenyloxy, aryloxy, heteroaryloxy, cycloalkyloxy, heterocycloalkyloxy, benzyloxy, haloalkoxy, haloalkenyloxy, haloaryloxy, halohetoraryloxy, nitro, nitroalkyl, nitroalkenyl, nitroalkynyl, nitroaryl, nitroheteroaryl, nitroheterocyclyoalkyl, amino, alkylamino, dialkylamino, alkenylamino, alkynylamino, arylamino, heteroarylamino, diarylamino, benzylamino, dibenzylamino, acyl, alkenylacyl, alkynylacyl, arylacyl, heteroarylacyl, acylamino, diacylamino, acyloxy, alkylsulphonyloxy, arylsulphonyloxy, heterocycloalkylamino, alkylsulphonyl, arylsulphonyl, carboalkoxy, carboaryloxy, alkylthio, benzylthio, acylthio, cyano, nitro, sulfate and phosphate;
n is 0-4, and
m is 0-5.

127. A method for the treatment of cancer as in claim 119, wherein R₄ has one of the following formulas: 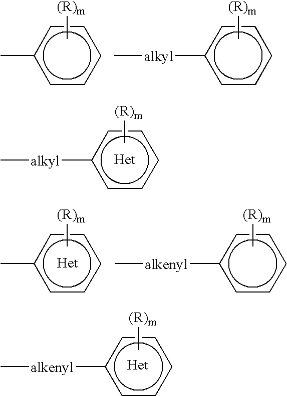
wherein each R is independently selected from the group consisting of alkyl, alkenyl, alkynyl, aryl, heteroaryl, cycloalkyl, heterocycloalkyl, halo, haloalkyl, haloalkenyl, haloalkynyl, haloaryl, haloheteroaryl, halocycloalkyl, haloheterocycloalkyl, hydroxy, alkoxy, alkenyloxy, aryloxy, heteroaryloxy, cycloalkyloxy, heterocycloalkyloxy, benzyloxy, haloalkoxy, haloalkenyloxy, haloaryloxy, halohetoraryloxy, nitro, nitroalkyl, nitroalkenyl, nitroalkynyl, nitroaryl, nitroheteroaryl, nitroheterocyclyoalkyl, amino, alkylamino, dialkylamino, alkenylamino, alkynylamino, arylamino, heteroarylamino, diarylamino, benzylamino, dibenzylamino, acyl, alkenylacyl, alkynylacyl, arylacyl, heteroarylacyl, acylamino, diacylamino, acyloxy, alklysulphonlyoxy, arylsulphonyloxy, heterocycloalkylamino, alkylsulphonyl, arylsulphnyl, carboalkoxy, carboaryloxy, alkylthio, benzylthio, acylthio, cyano, nitro, sulfate and phosphate;
and each m is from 0-5.

128. A method for the treatment of cancer as in claim 119, wherein R₅ is either H or optionally substituted alkyl.

129. A method for the treatment of cancer as in claim 119, wherein X is a carbonyl group.

130. A method for the treatment of cancer as in claim 129, wherein R₆ is either H or a nitrogen protecting group.

131. A method for the treatment of cancer as in claim 129, wherein R₇ is selected from the group consisting of optionally substituted alkyl optionally substituted alkenyl, optionally substituted alkynyl, optionally substituted aryl, optionally substituted cycloalkyl, optionally substituted heteroaryl, optionally substituted heterocycloalkyl, optionally substituted aryl alkyl, optionally substituted heteroaryl alkyl, optionally substituted cycloalkyl alkyl optionally substituted heterocycloalkyl alkyl, optionally substituted aryl alkenyl, optionally substituted hetero alkenyl, optionally substituted cycloalkyl alkenyl, optionally substituted heterocycloalkyl alkenyl, optionally substituted aryl alkynyl, optionally substituted heteroaryl alkynyl, optionally substituted cycloalkyl alkynyl, and optionally substituted heterocycloalkyl alkynyl.

132. A method for the treatment of cancer as in claim 131, wherein R₇ has one of the following formula: 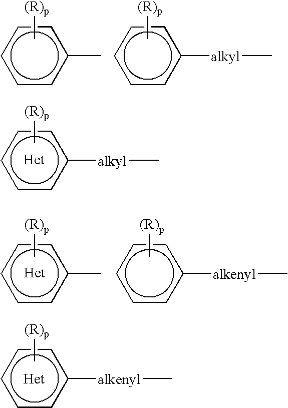
wherein each R is independently selected from the group consisting of alkyl alkenyl, alkynyl, aryl, heteroaryl, cycloalkyl, heterocycloalkyl, halo, haloalkyl, haloalkenyl, haloalkynyl, haloaryl, haloheteroaryl halocycloalkyl, haloheterocycloalkyl, hydroxy, alkoxy, alkenyloxy, aryloxy, heteroaryloxy, cycloalkyloxy, heterocycloalkyloxy, benzyloxy, haloalkoxy, haloalkenyloxy, haloaryloxy, halohetoraryloxy, nitro, nitroalkyl, nitroalkenyl, nitroalkynyl, nitroaryl, nitroheteroaryl, nitroheterocyclyoalkyl, amino, alkylamino, dialkylamino, alkenylamino, alkynylamino, arylamino, heteroarylamino, diarylamino, benzylamino, dibenzylamino, acyl, alkenylacyl, alkynylacyl, arylacyl, heteroarylacyl, acylamino, diacylamino, acyloxy, alklysulphonlyoxy, arylsulphonyloxy, heterocycloalkylamino, alkylsulphonyl, arylsulphonyl, carboalkoxy, carboaryloxy, alkylthio, benzylthio, acylthio, cyano, nitro, sulfate and phosphate;
and each p is from 0-5.

133. A method for the treatment of cancer as in claim 119, wherein the compound has a potency of cytotoxicity of IC₅₀≦10 μM against MM96 melanoma cells.

134. A method for the treatment of cancer as in claim 133, wherein the compound has a Selectivity Index of ≧1.5.

135. A method for the treatment of cancer as in claim 134, wherein the compound has a potency of IC₅₀≦1 μM against the MM96 melanoma cells and a Selectivity Index of ≧3.

136. A method for the treatment of cancer as in claim 135, wherein the compound has a potency of IC₅₀≦0.5 μM against the MM96 melanoma cells and a Selectivity Index of ≧4.

137. A method for the treatment of cancer as in claim 119, wherein the animal is a human.

138. A pharmaceutical composition containing one or more of the compounds of claim 84 and a pharmaceutically acceptable, carrier, diluent or excipient.

139. The use of any one or more of the compounds of claim 84 for the preparation of a medicament for the treatment of cancer.


SEQUENCE LISTING (4 sequences)

SEQ ID NO: 1 - DNA, 28 bp, Homo sapiens
attagcagcggaacaaggagtcagacat

SEQ ID NO: 2 - DNA, 28 bp, Homo sapiens
ctgtgaaagacacagaacagtacagggt

SEQ ID NO: 3 - DNA, 24 bp, Homo sapiens
ggctctccagaacatcatccctgc

SEQ ID NO: 4 - DNA, 24 bp, Homo sapiens
gggtgtcgctgttgaagtcagagg